(19) United States
(12) Patent Application Publication
(10) Pub. No.: US 2024/0301405 A1
(43) Pub. Date: Sep. 12, 2024

(54) GUIDE RNAS FOR CRISPR/CAS EDITING SYSTEMS

(71) Applicant: Beam Therapeutics Inc., Cambridge, MA (US)

(72) Inventors: Brian CAFFERTY, Cambridge, MA (US); Michael PACKER, Cambridge, MA (US); Yvonne ARATYN-SCHAUS, Cambridge, MA (US); Lo-I CHENG, Cambridge, MA (US)

(21) Appl. No.: 18/418,751

(22) Filed: Jan. 22, 2024

Related U.S. Application Data
(63) Continuation of application No. PCT/US2022/074041, filed on Jul. 22, 2022.
(60) Provisional application No. 63/225,322, filed on Jul. 23, 2021.
(60) Provisional application No. 63/255,927, filed on Oct. 14, 2021.

(51) Int. Cl. C12N 15/11; A61K 48/00; A61P 3/00; C12N 9/22

(52) U.S. Cl. CPC C12N 15/111; A61K 48/0008; A61P 3/00; C12N 9/22; C07K 2319/09; C12N 2310/20

(57) ABSTRACT

The present invention provides, among other things, a guide RNA conjugated to a NLS sequence (NLS-gRNA) and method for making and using the same. For example, in some embodiments, the 3′ end of the gRNA is conjugated to the N-terminus of a nuclear localization sequence (NLS) via a linker comprising a chemical moiety and a peptide spacer.

### CROSS-REFERENCE TO RELATED APPLICATIONS

This application claims priority to U.S. Provisional Patent Application Ser. Nos. 63/255,322 filed Jul. 23, 2021 and 63/255,927 filed Oct. 14, 2021, the contents of which are incorporated by reference herein in entirety for all purposes.

### INCORPORATION-BY-REFERENCE OF SEQUENCE LISTING

The contents of the file named “BEM-011WO_ST26.xml”, which was created on May 15, 2024, and is 60,965 bytes in size, is hereby incorporated by reference in its entirety.

### BACKGROUND

CRISPR/Cas editing systems include the use of guide RNA molecules (gRNA) in association with Cas endonucleases, and related enzymes, for applications in gene editing as well as related systems, including base editing. Briefly, one or more gRNA molecules assembles with a Cas protein in a complex and guides the ribonucleic acid complex (RNP) to specific DNA (for example, in Cas9 and Cas12 systems) and/or RNA (for example, in Cas13 systems) sequences.

A common form of gRNA used for therapeutic applications are single, non-natural RNAs of approximately 100 nucleotides that form ribonucleoproteins with Cas proteins such as Cas9. The ability to adapt CRISPR/Cas editing systems to new technologies (e.g., gene editing) requires that guide RNAs (gRNAs) persist long enough within target cells to enable desired editing. Degradation of gRNA by nucleases is a significant challenge to achieving desired editing. Additionally, gRNA needs to assemble into ribonucleic acid (RNP and be transported to the nucleus efficiently.

### SUMMARY OF THE INVENTION

Provided herein are methods, compositions and kits to enhance the potency of gRNA for use in CRISPR-Cas systems. The invention provides, in some aspects, methods to produce gRNA conjugated to an NLS sequence (NLS-gRNA) that has increased potency for use in CRISPR-Cas system, for example, increased frequency of successful editing events. The NLS-gRNA of the present invention can provide better trafficking of the gRNA to the nucleus to protect from cytosolic RNases and increase higher local concentration of gRNA for formation of RNP. NLS-gRNA of the present invention has significantly higher potency as compared to a counterpart gRNA without the NLS sequence and also shows a higher potency as compared to highly modified gRNAs.

In one aspect, the present invention provides, among other things, a guide RNA (gRNA) comprising a nuclear localization signal (NLS) linked to the gRNA through a linker, wherein the linker comprises a cysteine residue conjugated to the 3′ end of the gRNA.

In some embodiments, the linker comprises a cysteine residue at the N-terminus. In some embodiments, the linker comprises a cysteine residue at the C-terminus. In some embodiments, the linker comprises a cysteine residue at an internal site in the linker.

In some embodiments, the linker is conjugated to the 3′ end of the gRNA. In some embodiments, the linker is conjugated to the 5′ end of the gRNA. In some embodiments, the linker is conjugated to an internal region in the gRNA. In some embodiments, the linker is conjugated to a first hairpin region in the gRNA. In some embodiments, the linker is conjugated to a second hairpin region in the gRNA. In some embodiments, the linker is conjugated to a bulge region in the gRNA. In some embodiments, the gRNA comprises one or more modifications. In some embodiments, one or more modifications are 2′OMe modification. In some embodiments, one or more modifications comprise 2′-Fluoro modifications. In some embodiments, one or more modifications comprise phosphorothioate linkages.

In some embodiments, gRNA does not comprise a backbone modification. In some embodiments, one or more modifications occur at 1, 2, 3, 4, 5, 6, 7, 8, and 9 nucleotides from the 3′ end of the gRNA. In some embodiments, one or more modifications occur at 1, 2, 3, 4, 5, 6, 7, and 8 nucleotides from the 3′ end of the gRNA. In some embodiments, one or more modifications occur at 1, 2, 3, 4, 5, 6, and 7 nucleotides from the 3′ end of the gRNA. In some embodiments, one or more modifications occur at 1, 2, 3, 4, 5, and 6 nucleotides from the 3′ end of the gRNA. In some embodiments, one or more modifications occur at 1, 2, 3, 4, and 5 nucleotides from the 3′ end of the gRNA. In some embodiments, one or more modifications occur at 1, 2, 3, and 4 nucleotides from the 3′ end of the gRNA. In some embodiments, one or more modifications occur at 1, 2, and 3 nucleotides from the 3′ end of the gRNA. In some embodiments, one or more modifications occur at 1 and 2 nucleotides from the 3′ end of the gRNA. In some embodiments, one or more modifications occur at 1 nucleotide from the 3′ end of the gRNA.

In some embodiments, one or more modifications occur at 1, 2, 3, 4, 5, 6, 7, 8, and 9 nucleotides from the 5′ end of the gRNA. In some embodiments, one or more modifications occur at 1, 2, 3, 4, 5, 6, 7, and 8 nucleotides from the 5′ end of the gRNA. In some embodiments, one or more modifications occur at 1, 2, 3, 4, 5, 6, and 7 nucleotides from the 5′ end of the gRNA. In some embodiments, one or more modifications occur at 1, 2, 3, 4, 5, and 6 nucleotides from the 5′ end of the gRNA. In some embodiments, one or more modifications occur at 1, 2, 3, 4, and 5 nucleotides from the 5′ end of the gRNA. In some embodiments, one or more modifications occur at 1, 2, 3, and 4 nucleotides from the 5′end of the gRNA. In some embodiments, one or more modifications occur at 1, 2, and 3 nucleotides from the 5′ end of the gRNA. In some embodiments, one or more modifications occur at 1, and 2 nucleotides from the 5′ end of the gRNA. In some embodiments, one or more modifications occur at 1 nucleotide from the 5′ end of the gRNA

In some embodiments, more than 10% of the gRNA is modified. In some embodiments, more than 20% of the gRNA is modified. In some embodiments, more than 30% of the gRNA is modified. In some embodiments, more than 35% of the gRNA is modified. In some embodiments, more than 40% of the gRNA is modified. In some embodiments, more than 45% of the gRNA is modified. In some embodiments, more than 50% of the gRNA is modified. In some embodiments, more than 55% of the gRNA is modified. In some embodiments, more than 60% of the gRNA is modified. In some embodiments, more than 65% of the gRNA is modified. In some embodiments, more than 70% of the gRNA is modified. In some embodiments, more than 75% of the gRNA is modified. In some embodiments, more than 80% of the gRNA is modified. In some embodiments, more than 85% of the gRNA is modified. In some embodiments, more than 88% of the gRNA is modified. In some embodiments, more than 90% of the gRNA is modified. In some embodiments, more than 95% of the gRNA is modified.

In some embodiments, less than 10% of the gRNA is modified. In some embodiments, less than 20% of the gRNA is modified. In some embodiments, less than 30% of the gRNA is modified. In some embodiments, less than 35% of the gRNA is modified. In some embodiments, less than 40% of the gRNA is modified. In some embodiments, less than 45% of the gRNA is modified. In some embodiments, less than 50% of the gRNA is modified. In some embodiments, less than 55% of the gRNA is modified. In some embodiments, less than 60% of the gRNA is modified. In some embodiments, less than 65% of the gRNA is modified. In some embodiments, less than 70% of the gRNA is modified. In some embodiments, less than 75% of the gRNA is modified. In some embodiments, less than 80% of the gRNA is modified. In some embodiments, less than 85% of the gRNA is modified. In some embodiments, less than 88% of the gRNA is modified. In some embodiments, less than 90% of the gRNA is modified. In some embodiments, less than 95% of the gRNA is modified.

In some embodiments, the gRNA is conjugated to one or more NLS sequences. In some embodiments, the gRNA may comprise about or more than about 1, 2, 3, 4, 5, 6, 7, 8, 9, 10, or more NLSs at or near the 3′ end, about or more than about 1, 2, 3, 4, 5, 6, 7, 8, 9, 10, or more NLSs at or near the 5′ end, or a combination of these (e.g. one or more NLS at the 3′ end and one or more NLS at the 5′ end). When more than one NLS is present, each may be selected independently of the others, such that a single NLS may be present in more than one copy and/or in combination with one or more other NLSs present in one or more copies.

Non-limiting examples of NLSs include an NLS sequence derived from: the NLS of the SV40 virus large T-antigen, having the amino acid sequence PKKKRKV (SEQ ID NO: 41); the NLS from nucleoplasmin (e.g. the nucleoplasmin bipartite NLS with the sequence KRPAATKKAGQAKKKK (SEQ ID NO: 42)); the c-myc NLS having the amino acid sequence PAAKRVKLD (SEQ ID NO: 43) or RQRRNELKRSP (SEQ ID NO: 44); the hRNPA1 M9 NLS having the sequence NQSSNFGPMKGGNFGGRSSGPYGGGGQYFAKPRNQGGY (SEQ ID NO: 45); the sequence RMRIZFKNKGKDTAELRRRRVEVSVELRKAKKDEQILKRRNV (SEQ ID NO: 46) of the IBB domain from importin-alpha; the sequences VSRKRPRP (SEQ ID NO: 47) and PPKKARED (SEQ ID NO: 48) of the myoma T protein; the sequence POPKKKPL (SEQ ID NO: 49) of human p53; the sequence SALIKKKKKMAP (SEQ ID NO: 50) of mouse c-abl IV; the sequences DRLRR (SEQ ID NO: 51) and PKQKKRK (SEQ ID NO: 52) of the influenza virus NS1; the sequence RKLKKKIKKL (SEQ ID NO: 53) of the Hepatitis virus delta antigen; the sequence REKKKFLKRR (SEQ ID NO: 54) of the mouse Mx1 protein; the sequence KRKGDEVDGVDEVAKKKSKK (SEQ ID NO: 55) of the human poly(ADP-ribose) polymerase; and the sequence RKCLQAGMNLEARKTKK (SEQ ID NO: 56) of the steroid hormone receptors (human) glucocorticoid.

In some embodiments the NLS is derived from simian virus 40 (SV40). In some embodiments, the NLS comprises an amino acid sequence of KKKRKV (SEQ ID NO: 57). In some embodiments the NLS comprises a bipartite NLS. In some embodiments, the NLS comprises a bipartite NLS with SV40 NLS.

In some embodiments, the linker further comprises a peptide spacer. In some embodiments, the peptide spacer comprises more than 2 amino acids. In some embodiments, the peptide spacer comprises more than 3 amino acids. In some embodiments, the peptide spacer comprises more than 4 amino acids. In some embodiments, the peptide spacer comprises more than 5 amino acids. In some embodiments, the peptide spacer comprises more than 6 amino acids. In some embodiments, the peptide spacer comprises more than 7 amino acids. In some embodiments, the peptide spacer comprises more than 8 amino acids. In some embodiments, the peptide spacer comprises more than 9 amino acids. In some embodiments, the peptide spacer comprises more than 10 amino acids. In some embodiments, the peptide spacer comprises more than 12 amino acids. In some embodiments, the peptide spacer comprises more than 15 amino acids. In some embodiments, the peptide spacer comprises more than 18 amino acids. In some embodiments, the peptide spacer comprises more than 20 amino acids. In some embodiments, the peptide spacer comprises more than 25 amino acids. In some embodiments, the peptide spacer comprises more than 30 amino acids.

In some embodiments, the peptide spacer comprises 2-30 amino acids. In some embodiments, the peptide spacer comprises 5-25 amino acids. In some embodiments, the peptide spacer comprises 7-20 amino acids. In some embodiments, the peptide spacer comprises 7-15 amino acids. In some embodiments, the peptide spacer comprises 7-12 amino acids.

In some embodiments, the peptide spacer comprises about 5 amino acids. In some embodiments, the peptide spacer comprises about 7 amino acids. In some embodiments, the peptide spacer comprises about 8 amino acids. In some embodiments, the peptide spacer comprises about 9 amino acids. In some embodiments, the peptide spacer comprises about 10 amino acids. In some embodiments, the peptide spacer comprises about 11 amino acids. In some embodiments, the peptide spacer comprises about 12 amino acids. In some embodiments, the peptide spacer comprises about 13 amino acids. In some embodiments, the peptide spacer comprises about 14 amino acids. In some embodiments, the peptide spacer comprises about 15 amino acids.

In some embodiments, the peptide spacer comprises an amino acid sequence of KRTADGSEFESP (SEQ ID NO: 58). In some embodiments, the peptide spacer is 70% identical to amino acid sequence of KRTADGSEFESP. In some embodiments, the peptide spacer is 75% identical to amino acid sequence of KRTADGSEFESP. In some embodiments, the peptide spacer is 80% identical to amino acid sequence of KRTADGSEFESP. In some embodiments, the peptide spacer is 85% identical to amino acid sequence of KRTADGSEFESP. In some embodiments, the peptide spacer is 90% identical to amino acid sequence of KRTADGSEFESP. In some embodiments, the peptide spacer is 92% identical to amino acid sequence of KRTADGSEFESP. In some embodiments, the peptide spacer is 95% identical to amino acid sequence of KRTADGSEFESP. In some embodiments, the peptide spacer is 97% identical to amino acid sequence of KRTADGSEFESP. In some embodiments, the peptide spacer is 99% identical to amino acid sequence of KRTADGSEFESP.

In some embodiments, the linker further comprises a chemical moiety that conjugates gRNA to the peptide spacer or to the NLS.

In embodiments, gRNA is conjugated to NLS via a linker. In embodiments, said linker comprises a chemical moiety (e.g., L) and/or a peptidic moiety (e.g., a peptide spacer).

In embodiments, gRNA is conjugated to NLS directly via a chemical moiety (e.g., L). In embodiments, a chemical moiety (e.g., L) is non-peptidic. In embodiments, a chemical moiety (e.g., L) is covalently attached to both the gRNA and NLS.

In embodiments, gRNA is conjugated to NLS via a peptidic moiety (e.g., a peptide spacer). In embodiments, a peptidic moiety (e.g., a peptide spacer) is covalently attached to both the gRNA and NLS.

In embodiments, gRNA is conjugated to NLS via a linker comprising both a chemical moiety (e.g., L) and a peptidic moiety (e.g., a peptide spacer). In embodiments, such conjugates can have a structure according to Formula (I), where a chemical moiety L (e.g., a non-peptidic chemical moiety) is covalently attached to gRNA and a peptide spacer, and wherein the peptide spacer is covalently attached to NLS.

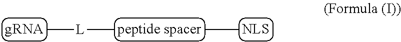

In embodiments, gRNA is conjugated to NLS via a chemical moiety (e.g., L) covalently attached to the C-terminus of the peptide spacer or the NLS amino acid sequence.

In embodiments, gRNA is conjugated to NLS via a chemical moiety (e.g., L) covalently attached to the N-terminus of the peptide spacer or the NLS amino acid sequence.

In embodiments, gRNA is conjugated to the peptide spacer or the NLS via a chemical moiety (e.g., L) covalently attached to the 3′ end of the gRNA.

In embodiments, gRNA is conjugated to the peptide spacer or the NLS via a chemical moiety (e.g., L) covalently attached to the 5′ end of the gRNA.

In embodiments, a chemical moiety (e.g., L) is covalently attached to a thiol-containing residue (e.g., a cysteine residue) of the peptide spacer or the NLS.

In embodiments, a chemical moiety (e.g., L) is covalently attached to a selenium-containing residue (e.g., a selenocysteine residue) of the peptide spacer or the NLS.

In embodiments, a chemical moiety (e.g., L) is covalently attached to an amino-containing residue (e.g., a lysine residue) of the peptide spacer or the NLS.

In embodiments, a chemical moiety (e.g., L) is covalently attached to a phenol-containing residue (e.g., a tyrosine residue) of the peptide spacer or the NLS.

In embodiments, amino acid residues used for formation of a linker (e.g., a thiol-, selenium-, amino-, or phenol-containing residue as described herein) comprise chemical modifications.

In some embodiments, the guide RNA further comprises a nucleic acid linker sequence. In some embodiments, the nucleic acid linker sequence is an RNA sequence.

In some embodiments, the nucleic acid linker sequence is positioned at the 5′ end and/or 3′ end of the guide RNA sequence.

In some embodiments, the nucleic acid linker comprises about 1-50 nucleotides. In some embodiments, the nucleic acid linker comprises about 1-45 nucleotides. In some embodiments, the nucleic acid linker comprises about 1-40 nucleotides. In some embodiments, the nucleic acid linker comprises about 1-35 nucleotides. In some embodiments, the nucleic acid linker comprises about 1-30 nucleotides. In some embodiments, the nucleic acid linker comprises about 1-25 nucleotides. In some embodiments, the nucleic acid linker comprises about 1-20 nucleotides. In some embodiments, the nucleic acid linker comprises about 1-15 nucleotides. In some embodiments, the nucleic acid linker comprises about 1-10 nucleotides. In some embodiments, the nucleic acid linker comprises about 1-5 nucleotides.

In some embodiments, the nucleic acid linker comprises about 5 nucleotides, about 10 nucleotides, about 15 nucleotides, about 20 nucleotides, about 25 nucleotides, about 30 nucleotides, about 35 nucleotides, about 40 nucleotides, about 45 nucleotides, or about 50 nucleotides.

In some embodiments, the guide RNA does not comprise a nucleic acid linker. In some embodiments, the nucleic acid linker comprises about one nucleotide. In some embodiments, the nucleic acid linker comprises about 2 nucleotides. In some embodiments, the nucleic acid linker comprises about 3 nucleotides. In some embodiments, the nucleic acid linker comprises about 4 nucleotides. In some embodiments, the nucleic acid linker comprises about 5 nucleotides. In some embodiments, the nucleic acid linker comprises about 6 nucleotides. In some embodiments, the nucleic acid linker comprises about 7 nucleotides. In some embodiments, the nucleic acid linker comprises about 8 nucleotides. In some embodiments, the nucleic acid linker comprises about 9 nucleotides. In some embodiments, the nucleic acid linker comprises about 10 nucleotides. In some embodiments, the nucleic acid linker comprises about 11 nucleotides. In some embodiments, the nucleic acid linker comprises about 12 nucleotides. In some embodiments, the nucleic acid linker comprises about 13 nucleotides. In some embodiments, the nucleic acid linker comprises about 14 nucleotides. In some embodiments, the nucleic acid linker comprises about 15 nucleotides. In some embodiments, the nucleic acid linker comprises about 16 nucleotides. In some embodiments, the nucleic acid linker comprises about 17 nucleotides. In some embodiments, the nucleic acid linker comprises about 18 nucleotides. In some embodiments, the nucleic acid linker comprises about 19 nucleotides. In some embodiments, the nucleic acid linker comprises about 20 nucleotides. In some embodiments, the nucleic acid linker comprises about 21 nucleotides. In some embodiments, the nucleic acid linker comprises about 22 nucleotides. In some embodiments, the nucleic acid linker comprises about 23 nucleotides. In some embodiments, the nucleic acid linker comprises about 24 nucleotides. In some embodiments, the nucleic acid linker comprises about 25 nucleotides.

In some embodiments, the nucleic acid linker comprises between about 50-100 nucleotides. In some embodiments, the nucleic acid linker comprises between about 100-150 nucleotides. In some embodiments, the nucleic acid linker comprises between about 150-200 nucleotides. In some embodiments, the nucleic acid linker comprises between about 200-500 nucleotides.

In some embodiments, the nucleic acid linker sequence is a linear linker sequence. In some embodiments, the linker sequence is a non-linear sequence. In some embodiments, the linker sequence comprises RNA secondary structures.

In some embodiments, the nucleic acid linker sequence is placed at the 3′ end and/or the 5′ end of the guide RNA sequence.

In some embodiments, the gRNA comprising the NLS improves base editing efficiency as compared to a gRNA without the NLS. In some embodiments, the gRNA comprising the NLS improves base editing efficiency by at least 1.5-fold as compared to a gRNA without the NLS. In some embodiments, the gRNA comprising the NLS improves base editing efficiency by at least 2-fold as compared to a gRNA without the NLS. In some embodiments, the gRNA comprising the NLS improves base editing efficiency by at least 2.5-fold as compared to a gRNA without the NLS. In some embodiments, the gRNA comprising the NLS improves base editing efficiency by at least 3-fold as compared to a gRNA without the NLS. In some embodiments, the gRNA comprising the NLS improves base editing efficiency by at least 4-fold as compared to a gRNA without the NLS. In some embodiments, the gRNA comprising the NLS improves base editing efficiency by at least 5-fold as compared to a gRNA without the NLS.

In some embodiments, the guide RNA further comprises a direct repeat sequence found in natural CRISPR systems.

In some embodiments, the gRNA is a single guide RNA (sgRNA). In some embodiments, the gRNA is a tracrRNA. In some embodiments, the gRNA is a crRNA.

In some embodiments, the guide RNA comprises a clustered regularly interspersed short palindromic repeats (CRISPR) RNA (crRNA). In some embodiments, the guide RNA further comprises a trans-activating RNA (tracrRNA).

In some embodiments, the crRNA is modified. In some embodiments, the tracrRNA is modified. In some embodiments, the crRNA and/or comprise chemically modified nucleotides. In some embodiments, the tracrRNA comprises additional sequences that maintain folding. In some embodiments, the linker comprises chemically modified nucleotides.

In some embodiments, the modifications to the crRNA, tracrRNA, and/or linker comprises one or more of 1) chemical modifications; 2) any nucleotide substitutions that preserve secondary structure; 3) alterations of the GC content; 4) addition of sequence to maintain predicted folding of tracrRNA.

In some embodiments provided herein is a method, wherein the NLS-gRNA is an extended guide RNA, or a Cas9 guide RNA, or a Cas13 guide RNA, or a Cas12 guide RNA such as Cas12a guide RNA, Cas12b guide RNA, Cas12c guide RNA, Cas12d guide RNA, Cas12e guide RNA, Cas12f guide RNA, Cas12g guide RNA, Cas12h guide RNA, Cas12i guide RNA, Cas12j guide RNA, Cas12k guide RNA. Accordingly, in some embodiments, the NLS-gRNA is an extended guide RNA. In some embodiments, the NLS-gRNA is a Cas9 guide RNA. In some embodiments, the NLS-gRNA is a Cas13 guide RNA. In some embodiments, the NLS-gRNA is a Cas12 guide RNA. In some embodiments, the NLS-gRNA is a Cas12a guide RNA. In some embodiments, the NLS-gRNA is a Cas12b guide RNA. In some embodiments, the NLS-gRNA is a Cas12c guide RNA. In some embodiments, the NLS-gRNA is a Cas12d guide RNA. In some embodiments, the NLS-gRNA is a Cas12e guide RNA. In some embodiments, the NLS-gRNA is a Cas12f guide RNA. In some embodiments, the NLS-gRNA is a Cas12g guide RNA. In some embodiments, the NLS-gRNA is a Cas12h guide RNA. In some embodiments, the NLS-gRNA is a Cas12i guide RNA. In some embodiments, the NLS-gRNA is a Cas12j guide RNA. In some embodiments, the NLS-gRNA is a Cas12k guide RNA.

In some embodiments, the NLS-gRNA comprises one or more of the following: a spacer, a lower stem, a bulge, an upper stem, a nexus and a hairpin.

In some embodiments, the stem loop comprises GC base pairs.

In some embodiments provided herein is a method, wherein the NLS-gRNA is produced at a yield of about 50%, 55%, 60%, 65%, 70%, 75%, 80%, 85%, 90%, 95%, 99% or more. Accordingly, in some embodiments, the NLS-gRNA is produced at a yield of about 50%. In some embodiments, the NLS-gRNA is produced at a yield of about 55%. In some embodiments, the NLS-gRNA is produced at a yield of about 60%. In some embodiments, the NLS-gRNA is produced at a yield of about 65%. In some embodiments, the NLS-gRNA is produced at a yield of about 70%. In some embodiments, the NLS-gRNA is produced at a yield of about 75%. In some embodiments, the NLS-gRNA is produced at a yield of about 80%. In some embodiments, the NLS-gRNA is produced at a yield of about 85%. In some embodiments, the NLS-gRNA is produced at a yield of about 90%. In some embodiments, the NLS-gRNA is produced at a yield of about 95%. In some embodiments, the NLS-gRNA is produced at a yield of more than 99%.

In some embodiments, the NLS-gRNA is produced at 50%, 55%, 60%, 65%, 70%, 75%, 80%, 85%, 90%, 95%, 99% or more improvement in yield as compared to conventional synthetic methods. Accordingly, in some embodiments, the NLS-gRNA is produced at 50% improvement in yield as compared to conventional synthetic methods. In some embodiments, the NLS-gRNA is produced at 55% improvement in yield as compared to conventional synthetic methods. In some embodiments, the NLS-gRNA is produced at 60% improvement in yield as compared to conventional synthetic methods. In some embodiments, the NLS-gRNA is produced at 65% improvement in yield as compared to conventional synthetic methods. In some embodiments, the NLS-gRNA is produced at 70% improvement in yield as compared to conventional synthetic methods. In some embodiments, the NLS-gRNA is produced at 75% improvement in yield as compared to conventional synthetic methods. In some embodiments, the NLS-gRNA is produced at 80% improvement in yield as compared to conventional synthetic methods. In some embodiments, the NLS-gRNA is produced at 85% improvement in yield as compared to conventional synthetic methods. In some embodiments, the NLS-gRNA is produced at 90% improvement in yield as compared to conventional synthetic methods. In some embodiments, the NLS-gRNA is produced at 95% improvement in yield as compared to conventional synthetic methods. In some embodiments, the NLS-gRNA is produced at 99% improvement in yield as compared to conventional synthetic methods. In some embodiments, the NLS-gRNA is produced at more than 99% improvement in yield as compared to conventional synthetic methods.

In some embodiments, the NLS-gRNA has a length of about 40 nucleotides, about 100 nucleotides, about 125 nucleotides, about 150 nucleotides, about 175 nucleotides, about 200 nucleotides, or greater than about 200 nucleotides. Accordingly, in some embodiments, the NLS-gRNA has a length of about 40 nucleotides. In some embodiments, the NLS-gRNA has a length of about 100 nucleotides. In some embodiments, the NLS-gRNA has a length of about 125 nucleotides. In some embodiments, the NLS-gRNA has a length of about 150 nucleotides. In some embodiments, the NLS-gRNA has a length of about 175 nucleotides. In some embodiments, the NLS-gRNA has a length of about 200 nucleotides. In some embodiments, the NLS-gRNA has a length of greater than about 200 nucleotides.

In some embodiments, the NLS-gRNA length is Cas dependent. For example, in some embodiments, the NLS-gRNA length for Cas12a is greater than 40 nucleotides. In some embodiments, the NLS-gRNA length for Cas9 is greater than 123 nucleotides. In some embodiments, the NLS-gRNA length for Cas9 is between 125-200 nucleotides. In some embodiments, the NLS-gRNA length for Cas9 is between 125-250 nucleotides. In some embodiments, the NLS-gRNA length for Cas9 is between 125-300 nucleotides. In some embodiments, the NLS-gRNA length for Cas9 is between 125-350 nucleotides. In some embodiments, the NLS-gRNA length for Cas9 is between 125-400 nucleotides. In some embodiments, the NLS-gRNA length for Cas9 is between 125-450 nucleotides. In some embodiments, the NLS-gRNA length for Cas9 is between 125-500 nucleotides.

In some embodiments, the NLS-gRNA comprises one or more backbone modifications.

In some embodiments, the one or more backbone modifications comprises a 2′ O-methyl or a phosphorothioate modification. Accordingly, in some embodiments, the one or more backbone modifications comprises a 2′ O-methyl modification. In some embodiments, the one or more backbone modifications comprises a phosphorothioate modification.

In some embodiments, the one or more backbone modifications is selected from 2′-O-methyl 3′-phosphorothioate, 2′-O-methyl, 2′-ribo 3′-phosphorothioate, 2′-fluoro, 2′-0-methoxyethyl morpholino (PMO), locked nucleic acid (LNA), deoxy, or 5′ phosphate modification. Accordingly, in some embodiments, the one or more backbone modifications comprises a 2′-O-methyl 3′-phosphorothioate modification. In some embodiments, the one or more backbone modifications comprises a 2′-O-methyl modification. In some embodiments, the one or more modifications comprises a 2′-ribo 3′-phosphorothioate modification. In some embodiments, the one or more modifications comprises a 2′-fluoro modification. In some embodiments, the one or more modifications comprises a 2′-O-methoxyethyl morpholino (PMO). In some embodiments, the one or more modifications comprises a locked nucleic acid (LNA). In some embodiments, the one or more modifications comprises a deoxy modification. In some embodiments, the one or more modifications comprises a 5′ phosphate modification.

Various modified RNA bases are known in the art and include for example, 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, fluoro C, fluoro U, fluoro A, fluoro G bases. Various 2′-OMethyl modifications can also be used with the methods described herein. For example, the following RNA comprising one or more of the following 2′-OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the RNA comprises one or more of the following modifications: phosphorothioates, 2′O-methyls, 2′ fluoro (2′F), deoxy. In some embodiments, the RNA comprises 2′OMe modifications at the 3′ end. In some embodiments, the RNA comprises 2′OMe modifications at the 5′ end. In some embodiments, the RNA comprises 2′OMe modifications at the 3′ end and 5′ end. In some embodiments, the RNA comprises one or more of the following modifications: 2′-O-2-Methoxyethyl (MOE), locked nucleic acids, bridged nucleic acids, unlocked nucleic acids, peptide nucleic acids, morpholino nucleic acids. In some embodiments, the RNA comprises one or more of the following base modifications: 2,6-diaminopurine, 2-aminopurine, pseudouracil, NI-methyl-pseudouracil, 5′ methyl cytosine, 2′ pyrimidinone (zebularine), thymine. Other modified bases include for example, 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Deoxy-r(A,C,G,U), 3′-O-Methyl-r(A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the RNA can comprise a modified base such as, for example, 5′ Int, 3′ Azide (NHS Ester); 5′ Hexynyl; 5′ Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

In some embodiments, the method produces NLS-gRNA at a purity of about 50%, 60%, 70%, 80%, 90%, or more than 90%. Accordingly, in some embodiments, the method produces NLS-gRNA at a purity of about 50%. In some embodiments, the method produces NLS-gRNA at a purity of about 60%. In some embodiments, the method produces NLS-gRNA at a purity of about 70%. In some embodiments, the method produces NLS-gRNA at a purity of about 80%. In some embodiments, the method produces NLS-gRNA at a purity of about 90%. In some embodiments, wherein the method produces NLS-gRNA at a purity of about 91%, about 92%, about 93%, about 94%, about 95%, about 96%, about 97%, about 98%, about 99%, or more than 99%. In some embodiments, the method produces NLS-gRNA at a purity of about 91%. In some embodiments, the method produces NLS-gRNA at a purity of about 92%. In some embodiments, the method produces NLS-gRNA at a purity of about 93%. In some embodiments, the method produces NLS-gRNA at a purity of about 94%. In some embodiments, the method produces NLS-gRNA at a purity of about 95%. In some embodiments, the method produces NLS-gRNA at a purity of about 96%. In some embodiments, the method produces NLS-gRNA at a purity of about 97%. In some embodiments, the method produces NLS-gRNA at a purity of about 98%. In some embodiments, the method produces NLS-gRNA at a purity of about 99%. In some embodiments, the method produces NLS-gRNA at a purity of greater than about 99%.

In one aspect, the present invention provides, among other things, a composition comprising a guide RNA (gRNA) comprising a nuclear localization signal (NLS) linked to the gRNA through a linker, wherein the linker comprises a cysteine residue conjugated to the 3′ end of the gRNA, wherein the NLS-guide RNA is encapsulated in a lipid nanoparticle (LNP). In one aspect, the present invention provides, among other things, a composition comprising a guide RNA (gRNA) comprising a nuclear localization signal (NLS) linked to the gRNA through a linker, wherein the linker comprises a cysteine residue conjugated to the 3′ end of the gRNA, wherein the NLS-guide RNA is associated with lipid nanoparticle (LNP).

In some embodiments, the composition comprises a nuclease. In some embodiments, the composition comprises a nucleic acid encoding a nuclease. In some embodiments, the composition comprises an mRNA encoding a nuclease.

In some embodiments, the nuclease is conjugated to a NLS. In some embodiments, the Cas protein is conjugated to a NLS. In some embodiments, the Cas protein does not comprise a NLS. In some embodiments, the Cas protein is not conjugated to a NLS. In some embodiments, the Cas9 protein does not comprise a NLS. In some embodiments, the Cas9 protein is not conjugated to a NLS.

In some embodiments, the composition comprises a NLS-gRNA and an mRNA encoding a nuclease. In some embodiments, the composition comprises a NLS-gRNA and an mRNA encoding a nuclease at 1:1 weight ratio. In some embodiments, the composition comprises a NLS-gRNA and an mRNA encoding a nuclease at 2:1 weight ratio. In some embodiments, the composition comprises a NLS-gRNA and an mRNA encoding a nuclease at 3:1 weight ratio. In some embodiments, the composition comprises a NLS-gRNA and an mRNA encoding a nuclease at 4:1 weight ratio. In some embodiments, the composition comprises a NLS-gRNA and an mRNA encoding a nuclease at 5:1 weight ratio. In some embodiments, the composition comprises a NLS-gRNA and an mRNA encoding a nuclease at 6:1 weight ratio. In some embodiments, the composition comprises a NLS-gRNA and an mRNA encoding a nuclease at 7:1 weight ratio. In some embodiments, the composition comprises a NLS-gRNA and an mRNA encoding a nuclease at 8:1 weight ratio. In some embodiments, the composition comprises a NLS-gRNA and an mRNA encoding a nuclease at 9:1 weight ratio. In some embodiments, the composition comprises a NLS-gRNA and an mRNA encoding a nuclease at 10:1 weight ratio. In some embodiments, the composition comprises a NLS-gRNA and an mRNA encoding a nuclease at 12:1 weight ratio. In some embodiments, the composition comprises a NLS-gRNA and an mRNA encoding a nuclease at 15:1 weight ratio.

In some embodiments, the nuclease is a CRISPR class 2 type II enzyme. In some embodiments, the nuclease is a CRISPR class 2 type V enzyme. In some embodiments, the nuclease CRISPR class 2 type VI enzyme. In some embodiments, wherein the nuclease is a Cas9, Cpf1, SaCas9, Cas12, Cas13, or modified versions thereof. Accordingly, in some embodiments, the nuclease is a Cas9, or modified versions thereof. In some embodiments, the nuclease is a Cpf1, or modified versions thereof. In some embodiments, nuclease is a Staphylococcus aureus Cas9 (SaCas9), or modified versions thereof. In some embodiments, nuclease is a Streptococcus thermophilus 1 Cas9 (St1Cas9) or modified versions thereof. In some embodiments, nuclease is a Streptococcus pyogenes Cas9 (SpCas9), or modified versions thereof. In some embodiments, nuclease is a Cas12, or modified versions thereof. In some embodiments, the nuclease is a Cas13, or modified versions thereof.

In some embodiments, the Cas9 comprises a nuclease dead Cas9 (dCas9). In some embodiments, the Cas9 comprises a Cas9 nickase (nCas9). In some embodiments, the Cas9 comprises a nuclease active Cas9.

In some embodiments, the nuclease domain is fused to a heterologous polypeptide. In some embodiments the heterologous polypeptide includes an effector domain that is capable of making a modification to a nucleic acid (e.g., DNA). For example, the DNA effector domain may be a deaminase domain, such as a cytidine deaminase domain, cytosine domain or an adenosine deaminase domain. In certain embodiments, the deaminase domain is a cytidine deaminase domain, such as an APOBEC or AID cytidine deaminase. For base editing proteins that are capable of deaminating a cytidine to a uridine, e.g., to induce a C to T mutation in a DNA molecule, the cytidine deaminase can be a deaminase from the apolipoprotein B mRNA-editing complex (APOBEC) family deaminase. In some embodiments, the heterologous polypeptide is a cytidine or cytosine deaminase domain. In some embodiments, the heterologous polypeptide is a cytosine deaminase domain. In some embodiments, the heterologous polypeptide is a cytidine deaminase domain. In some embodiments, the heterologous polypeptide is an adenosine or adenine deaminase domain. In some embodiments, the heterologous polypeptide is an adenosine domain. In some embodiments, the heterologous polypeptide is an adenine domain.

In some embodiments, a heterologous polypeptide is an adenosine deaminase variant domain. In some embodiments, the adenosine deaminase variant domain comprises one or more mutations with reference to SEQ ID NO: 3. In some embodiments, the adenosine deaminase variant domain comprises V82G. In some embodiments, the adenosine deaminase variant domain comprises Y147T/D. In some embodiments, the adenosine deaminase variant domain comprises Q154S. In some embodiments, the adenosine deaminase variant domain comprises L36H. In some embodiments, the adenosine deaminase variant domain comprises I76Y. In some embodiments, the adenosine deaminase variant domain comprises F149Y. In some embodiments, the adenosine deaminase variant domain comprises N157K. In some embodiments, the adenosine deaminase variant domain comprises V82G, Y147T/D and Q154S. In some embodiments, the adenosine deaminase variant domain comprises V82G, Y147T/D, Q154S, and L36H. In some embodiments, the adenosine deaminase variant domain comprises V82G, Y147T/D, Q154S, and I76Y. In some embodiments, the adenosine deaminase variant domain comprises V82G, Y147T/D, Q154S, and F149Y. In some embodiments, the adenosine deaminase variant domain comprises V82G, Y147T/D, Q154S, and N157K. In some embodiments, the adenosine deaminase variant domain comprises V82G, Y147T/D, Q154S, and D167N. In some embodiments, the adenosine deaminase variant domain comprises V82G, Y147T/D, Q154S, and one or more of L36H, I76Y, F149Y, N157K, and D167N. In some embodiments, the adenosine deaminase domain comprises mutations I76Y, V82G, Y147T, and Q154S. In some embodiments, the adenosine deaminase domain comprises mutations L36H, V82G, Y147T, Q154S, and N157K. In some embodiments, the adenosine deaminase domain comprises mutations V82G, Y147D, F149Y, Q154S, and D167N. In some embodiments, the adenosine deaminase domain comprises mutations L36H, V82G, Y147D, F149Y, Q154S, N157K, and D167N. In some embodiments, the adenosine deaminase domain comprises mutations L36H, I76Y, V82G, Y147T, Q154S, and N157K. In some embodiments, the adenosine deaminase domain comprises mutations I76Y, V82G, Y147D, F149Y, Q154S, and D167N. In some embodiments, the adenosine deaminase domain comprises mutations Y147D, F149Y, and D167N. In some embodiments, the adenosine deaminase domain comprises mutations L36H, I76Y, V82G, Q154S, and N157K. In some embodiments, the adenosine deaminase domain comprises mutations I76Y, V82G, and Q154S. In some embodiments, the adenosine deaminase domain comprises mutations L36H, I76Y, V82G, Y147D, F149Y, Q154S, N157K, and D167N.

In some embodiments, a heterologous polypeptide is fused to the N-terminus of a nuclease domain. In some embodiments, a heterologous polypeptide is fused to the C-terminus of a nuclease domain. In some embodiments, a heterologous polypeptide is internal to a nuclease domain. In some embodiments, a heterologous polypeptide is fused to the N-terminus of Cas9. In some embodiments, a heterologous polypeptide is fused to the C-terminus of Cas9. In some embodiments, a heterologous polypeptide is internal to Cas9. In some embodiments, an adenosine deaminase variant is fused to the N-terminus of Cas9. In some embodiments, an adenosine deaminase variant is fused to the C-terminus of Cas9. In some embodiments, an adenosine deaminase variant is internal to Cas9.

In some embodiments, the NLS-gRNA is suitable for use with CRISPR/Cas systems. In some embodiments, the NLS-gRNA is suitable for use with CRISPR class 2 type II enzymes. In some embodiments, the NLS-gRNA is suitable for use with CRISPR class 2 type V enzymes. In some embodiments, the NLS-gRNA is suitable for use with CRISPR class 2 type VI enzymes. In some embodiments, wherein the NLS-gRNA is suitable for use with Cas9, Cpf1, SaCas9, Cas12, Cas13, or modified versions thereof. Accordingly, in some embodiments, the NLS-gRNA is suitable for use with Cas9, or modified versions thereof. In some embodiments, the NLS-gRNA is suitable for use with Cpf1, or modified versions thereof. In some embodiments, the NLS-gRNA is suitable for use with SaCas9, or modified versions thereof. In some embodiments, the NLS-gRNA is suitable for use with Cas12, or modified versions thereof. In some embodiments, the NLS-gRNA is suitable for use with Cas13, or modified versions thereof. In some embodiments, the NLS-gRNA is in complex with the Cas enzyme.

In some embodiments, RNA sequences are included that will be cleaved by the endonuclease activity of some Cas e.g. Cas12a and Cas13 to linearize gRNA prior to or during assembly with Cas protein.

In some embodiments, the NLS-gRNA provides increased stability and resistance to cellular exonucleases in comparison to gRNA without the NLS sequence. In some embodiments, the NLS-gRNA provides increased editing events in target cells using a CRISPR/Cas editing system.

In some embodiments, the NLS-gRNA is in a complex with a CRISPR class 2 type II enzyme. In some embodiments, the NLS-gRNA is in a complex with a CRISPR class 2 type V enzyme. In some embodiments, the NLS-gRNA is in a complex with a CRISPR class 2 type VI enzyme. In some embodiments, the NLS-gRNA is in a complex with Cas9, Cpf1, SaCas9, Cas12, Cas13, or modified versions thereof.

In some aspects, a Cas protein complex is provided, the complex comprising a Cas nuclease and a NLS-gRNA.

In some embodiments, the Cas nuclease is a CRISPR class 2 type II enzyme. In some embodiments, the Cas nuclease is a CRISPR class 2 type V enzyme. In some embodiments, the Cas nuclease is a CRISPR class 2 type VI enzyme. In some embodiments, the Cas nuclease is selected from Cas9, Cpf1, SaCas9, Cas12, Cas13, or modified versions thereof.

In some embodiments, provided herein is a method for targeted transcription activation, targeted transcription repression, targeted epigenome modification, or targeted genome modification, the method comprising introducing into a eukaryotic cell: (a) a NLS-conjugated guide RNA (NLS-gRNA), (b) at least one CRISPR/Cas protein or a nucleic acid encoding at least one CRISPR/Cas protein, wherein interactions between (a) and (b) and a target sequence in chromosomal DNA leads to targeted transcription activation, targeted transcription repression, targeted epigenome modification, or targeted genome modification.

In some embodiments, provided herein is a method for targeted RNA modification, the method comprising introducing into a eukaryotic cell: (a) a NLS-conjugated guide RNA (NLS-gRNA) and (b) at least one CRISPR/Cas protein or a nucleic acid encoding the at least one CRISPR/Cas protein, wherein interactions between (a) and (b) and an RNA expressed by chromosomal DNA leads to a modification of the RNA expressed by the chromosomal DNA.

In some embodiments, the RNA expressed by the chromosomal DNA is a messenger RNA (mRNA).

In some aspects, the present invention provides a pharmaceutical composition comprising the NLS-gRNA of the present invention and a pharmaceutically acceptable carrier.

In one aspect, the present invention provides, among other things, a composition comprising an engineered or non-naturally occurring CRISPR associated Cas (CRISPR-Cas) system comprising: a Cas protein, a gRNA comprising a nuclear localization signal (NLS) linked to the gRNA through a linker, wherein the linker comprises a cysteine residue conjugated to the 3′ end of the gRNA; and wherein the gRNA is capable of forming a complex with a Cas protein and targeting the Cas9 protein to a target DNA.

In some embodiments, the gRNA comprises a nucleic acid sequence: 5′-CAGUAUGGACACUGUCCAAA-3′ (SEQ ID NO: 2).

In one aspect, the present invention provides, among other things, a composition comprising an engineered or non-naturally occurring CRISPR associated Cas (CRISPR-Cas) system comprising: (a) a saCas9 protein; (b) an adenosine deaminase variant fused to the Cas9 protein; and (c) a gRNA comprising a nuclear localization signal (NLS) linked to the gRNA through a linker; wherein the linker comprises a cysteine residue conjugated to the 3′ end of the gRNA; and wherein the gRNA is capable of forming a complex with a saCas9 protein and targeting the saCas9 protein to a target DNA; wherein the adenosine deaminase variant comprises V82G, Y147T/D, Q154S, and one or more of L36H, I76Y, F149Y, N157K, and D167N with reference to SEQ ID NO: 3; and wherein the gRNA comprises SEQ ID NO: 2.

In one aspect, the present invention provides, among other things, a method of treating a genetic disease in a subject in need thereof by administering to the subject the composition of the present invention (e.g., NLS-gRNA).

In one aspect, the present invention provides, among other things, a method of treating Glycogen Storage Disease Type 1a (GSD1a), the method comprising administering to the subject the composition of the present invention (e.g., NLS-gRNA).

In some embodiments, provided herein is a composition comprising gRNA conjugated to NLS, wherein the nuclear delivery of the composition is increased by about 2 to 5 fold relative to a composition comprising gRNA without NLS. In some embodiments, the nuclear delivery of the composition is increased by about 2 fold relative to a composition comprising gRNA without NLS. In some embodiments, the nuclear delivery of the composition is increased by about 3 fold relative to a composition comprising gRNA without NLS. In some embodiments, the nuclear delivery in increased by about 4 fold relative to a composition comprising gRNA without NLS. In some embodiments, the nuclear delivery in increased by about 5 fold relative to a composition comprising gRNA without NLS. In some embodiments, the nuclear delivery in increased by greater than about 2 fold relative to a composition comprising gRNA without NLS. In some embodiments, the nuclear delivery in increased by 1.5 to 10 fold relative to a composition comprising gRNA without NLS. In some embodiments, the nuclear delivery in increased by greater than about 10 fold relative to a composition comprising gRNA without NLS.

In some embodiments, the gRNA comprises a sequence with 70%, 80%, 90%, 95%, 99% or 100% identity to any one of sequences in Table 8. In some embodiments, the gRNA comprises a sequence with 70% identity to any one of sequences in Table 8. In some embodiments, the gRNA comprises a sequence with 75% identity to any one of sequences in Table 8. In some embodiments, the gRNA comprises a sequence with 80% identity to any one of sequences in Table 8. In some embodiments, the gRNA comprises a sequence with 85% identity to any one of sequences in Table 8. In some embodiments, the gRNA comprises a sequence with 90% identity to any one of sequences in Table 8. In some embodiments, the gRNA comprises a sequence with 95% identity to any one of sequences in Table 8. In some embodiments, the gRNA comprises a sequence with 99% identity to any one of sequences in Table 8. In some embodiments, the gRNA comprises a sequence with 100% identity to any one of sequences in Table 8.

In some embodiments, provided herein is a composition comprising gRNA conjugated to NLS, wherein gene editing efficiency is increased by about 2 to 5 fold relative to gRNA without NLS. In some embodiments, the gene editing efficiency is increased by about 2 fold relative to gRNA without NLS. In some embodiments, the gene editing efficiency is increased by about 3 fold relative to gRNA without NLS. In some embodiments, the gene editing efficiency is increased by about 4 fold relative to gRNA without NLS. In some embodiments, the gene editing efficiency is increased by about 5 fold relative to gRNA without NLS. In some embodiments, the gene editing efficiency is increased by about 1.5 to 10 fold relative to gRNA without NLS.

In some embodiments, the gRNA target sequence has 70%, 80%, 90%, 95%, 99% or 100% identity to SEQ ID NO: 17. In some embodiments, the gRNA target sequence has 70% identity to SEQ ID NO: 17. In some embodiments, the gRNA target sequence has 75% identity to SEQ ID NO: 17. In some embodiments, the gRNA target sequence has 70%, 80% identity to SEQ ID NO: 17. In some embodiments, the gRNA target sequence has 85% identity to SEQ ID NO: 17. In some embodiments, the gRNA target sequence has 90% identity to SEQ ID NO: 17. In some embodiments, the gRNA target sequence has 95% identity to SEQ ID NO: 17. In some embodiments, the gRNA target sequence has 100% identity to SEQ ID NO: 17.

In some embodiments, the gRNA targets one or more of organs selected from liver, kidney, brain and heart. In some embodiments, the gRNA targets liver.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another, in some embodiments, two or more entities that are physically associated with one another are not covalently linked to one another but are non-covalently associated, for example by means of hydrogen bonds, van der Waals interaction, hydrophobic interactions, magnetism, and combinations thereof.

“Adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxy inosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA). In some embodiments, the target polynucleotide is single- or double-stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA.

“Adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

“Adenosine Base Editor (ABE)” is meant a base editor comprising an adenosine deaminase.

“Adenosine Base Editor 8 (ABE8) polypeptide” or “ABE8” is meant a base editor as defined herein comprising an adenosine deaminase variant comprising an alteration at amino acid position 82 and/or 166 of the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO:3). In some embodiments, ABE8 comprises further alterations, as described herein, relative to the reference sequence.

“Adenosine Base Editor 8 (ABE8) polynucleotide” is meant a polynucleotide encoding an ABE8 polypeptide.

“Adenosine Deaminase polynucleotide” is meant a polynucleotide encoding an adenosine deaminase polypeptide. In particular embodiments, the adenosine deaminase polynucleotide encodes an adenosine deaminase variant comprising V82G, Y147T/D, Q154S, and one or more of L36H, I76Y, F149Y, N157K, and D167N. In some embodiments, the adenosine deaminase polynucleotide encodes an adenosine deaminase variant comprising one of the following combinations of alterations: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+176Y+V82G+Y147D+F149Y+Q154S+N157K+D167N.

In some embodiments, the deaminase or deaminase domain is a variant of a naturally occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase. In some embodiments, the adenosine deaminase is from a bacterium, such as, E. coli, S. aureus, B. subtilis, S. typhi, S. putrefaciens, H. influenzae, C. crescentus, or G. sulfurreducens.

In some embodiments, the adenosine deaminase is a TadA deaminase. In some embodiments, the TadA deaminase is an E. coli TadA (ecTadA) deaminase or a fragment thereof. In some embodiments, the ecTadA deaminase is truncated ecTadA. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 16, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 16, 17, 18, 19, or 20 C-terminal amino acid residues relative to the full length ecTadA. In some embodiments, the ecTadA deaminase does not comprise an N-terminal methionine. In some embodiments, the TadA deaminase is an N-terminal truncated TadA. In particular embodiments, the TadA is any one of the TadAs described in PCT/US2017/045381, which is incorporated herein by reference in its entirety.

In some embodiments, the TadA deaminase is TadA variant. In some embodiments, the TadA variant is TadA*7.10 comprising V82G, Y147T/D, Q154S, and one or more of L36H, I76Y, F149Y, N157K, and D167N. In some embodiments, the TadA variant is TadA*7.10 comprising a combination of alterations selected from among the following: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+176Y+V82G+Y147D+F149Y+Q154S+N157K+D167N. In some embodiments, the TadA variant is MSP605, MSP680, MSP823, MSP824, MSP825, MSP827, MSP828, or MSP829.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a nitrogenous base within DNA. In some embodiments, the base editor is capable of deaminating a nitrogenous base within RNA. In some embodiments, the base editor is capable of deaminating a ribonucleoside. In some embodiments, the base editor is capable of deaminating a deoxyribonucleoside. In some embodiments, the base editor is capable of deaminating a cytosine. In some embodiments, the base editor is capable of deaminating a cytidine. In some embodiments, the base editor is capable of deaminating an adenosine. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments, the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

Base Editing Activity: By “base editing activity” is meant acting to chemically alter a base within a polynucleotide (e.g., by deaminating the base). In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C. In another embodiment, the base editing activity is cytidine deaminase activity, e.g., converting target C·G to T·A and adenosine or adenine deaminase activity, e.g., converting A·T to G·C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: By “complementary” or “complementarity” is meant that a nucleic acid can form hydrogen bond(s) with another nucleic acid sequence by either traditional Watson-Crick or Hoogsteen base pairing. Complementary base pairing includes not only G-C and A-T base pairing, but also includes base pairing involving universal bases, such as inosine. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being base paired to a second nucleic acid sequence having 10 nucleotides represents 50%, 60%, 70%, 80%, 90%, and 100% complementarity respectively). To determine percent complementarity, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being base paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers. In some embodiments, the CRISPR array includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity and/or that binds to a target site on a nucleic acid specified by a RNA guide. In different embodiments, a CRISPR effector has endonuclease activity, nickase activity, exonuclease activity, transposase activity, and/or excision activity. In other embodiments, the CRISPR effector is nuclease inactive.

crRNA: The term “CRISPR RNA” or “crRNA,” as used herein, refers to a RNA molecule including a guide sequence used by a CRISPR effector to target a specific nucleic acid sequence. Typically, crRNAs contain a sequence that mediates target recognition and a sequence that forms a duplex with a tracrRNA. In some embodiments, the crRNA: tracrRNA duplex binds to a CRISPR effector.

Duplex: As used herein, “duplex” refers to a double helical structure formed by the interaction of two single stranded nucleic acids. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA, adenine (A) forms a base pair with thymine (T) in DNA, and adenine (A) forms a base pair with uracil (U) in RNA. Conditions under which base pairs can form include physiological or biologically relevant conditions (e.g., intracellular: pH 7.2, 140 mM potassium ion; extracellular pH 7.4, 145 mM sodium ion). Furthermore, duplexes are stabilized by stacking interactions between adjacent nucleotides. As used herein, a duplex may be established or maintained by base pairing or by stacking interactions. A duplex is formed by two complementary nucleic acid strands, which may be substantially complementary or fully complementary. Single-stranded nucleic acids that base pair over a number of bases are said to “hybridize.”

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Hybridize: By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Hybridization occurs by hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Indel: As used herein, the term “indel” refers to insertion or deletion of bases in a nucleic acid sequence. It commonly results in mutations and is a common form of genetic variation.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Linker or Spacer: The linker or spacer is a nucleotide or amino acid sequence that physically separates the terminal positions of the gRNA sequence from the NSL sequence to enable Cas binding and function of the gRNA. In some embodiments, the linker is RNA. In some embodiments, the linker is a chemical moiety. In some embodiments, the linker is a peptide. In some embodiments, the linker is DNA. In some embodiments, the linker is a chemical linker, for example, PEG9/18. In some embodiments, the linker is a DNA linker.

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. A PAM may be required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used interchangeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term “RNA guide” or “guide RNA” refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides. The term “RNA guide” or “guide RNA” also refers to NLS-gRNA.

Single Strand Ligase: As used herein, the term “Single Strand Ligase” means a ligase that does not require an oligonucleotide splint or a template for its ligating activity.

Splint or Oligonucleotide Splint: The terms “splint” or “oligonucleotide splint” refers to a single stranded RNA or DNA or other polymer that is capable of hybridizing with at least two, three or more single stranded RNA nucleotides. For example, the splint can refer to an oligonucleotide splint.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA,” “single guide RNA,” or “guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may include coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, or combination with other pharmaceutical agents. Also, the specific therapeutically effective amount (and/or unit dose) for any particular subject may depend upon a variety of factors including the disorder being treated and the severity of the disorder, the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1 is an exemplary schematic of gRNA conjugated to an NLS sequence. In this particular design, the 3′ end of the gRNA is conjugated to the N-terminus of a peptide spacer followed by an NLS sequence derived from SV40.

FIG. 2 is an exemplary graph that shows results of adenine to guanine base (A-to-G) conversion percentage achieved with a base editor comprising an adenine deaminase fused to the N-terminus of a spCas9. A-to-G conversion percentage (y-axis) is plotted for various guide RNAs with or without NLS at various ratios of mRNA encoding a base editor (1:1, 1:3, and 1:9). “Lipo Control” comprises an mRNA encoding a base editor gRNA (without NLS) in lipofectamine. “Lipo Control” was formulated to serve as a transfection control against the LNP group.

FIG. 3A is an exemplary schematic of gRNA with different modifications. “EM” (end-modified) gRNAs have 3 nucleotides at both 3′ and 5′ ends with 2′OMe modifications. “HM1” (heavy modified 1) has 47% of gRNA modified with 2′OMe modification. “HM2” (heavy modified 2) has 60% of gRNA modified with 2′OMe modification. “HM3” (heavy modified 3) has 88% of gRNA modified with 2′OME and 2′F modifications. The NLS-gRNA used in Example 2 comprises end-modifications. FIG. 3B is an exemplary graph that shows results of adenine to guanine base (A-to-G) conversion percentage achieved in mice with a base editor comprising an adenine deaminase fused to the N-terminus of a spCas9. A-to-G conversion percentage (y-axis) plotted for various guide RNAs with or without NLS, and with or without various modifications in gRNA.

FIG. 4A is an exemplary graph that shows results of base editing efficiency achieved in non-human primates (NHPs) with a base editor comprising an adenine deaminase fused to the N-terminus of a spCas9. Base editing efficiency in liver (y-axis) is plotted for various guide RNAs with or without NLS, and with or without various modifications in gRNA. FIG. 4B is a series of exemplary graphs that shows toxicology results. AST and ALT levels were measured 24 hour-post administration and fold change as compared to AST/ALT levels prior to administration with formulations comprising different gRNAs is shown.

FIG. 5 is an exemplary graph that shows results of adenine to guanine base (A-to-G) conversion percentage achieved in mice with a base editor comprising an adenine deaminase fused to the N-terminus of a saCas9. A-to-G conversion percentage (y-axis) for both on-target and bystander editing was plotted for various guide RNAs with various purity and modifications.

FIGS. 6A and 6B depict in vivo correction of GSD1a mutations in liver extracts of transgenic mouse models heterozygous for huG6PC-R83C. FIG. 6A is a schematic depicting in vivo workflow. Lipid nanoparticles (LNP) carrying base editor mRNA and gRNA were dosed via IV injection in transgenic mice heterozygous for huG6PC (huR83C HET), harboring the R83C mutation. FIG. 6B is a bar graph depicting A-to-G base editing efficiency of the GSD1a R83C mutation using MSP828 comparing on-target to bystander editing.

FIG. 6C is a bar graph depicting correction of the GSD1a R83C mutation in a transgenic mouse model heterozygous for huG6PC, harboring the R83C mutation, using TadA adenosine deaminase variants MSP605, MSP824, MSP825, MSP680, MSP828, and MSP820. In vitro screens were run to select desirable base-editors for R83C correction. LNP co-formulations of gRNA and representative base-editors were dosed (at a sub-saturating dose of 1 mpk), in vivo, in transgenic mice heterozygous for huG6PC-R83C. The base-editing potency of the variants for the R83C correction in livers of the LNP-treated, huG6PC-R83C heterozygote, transgenic animals are shown in FIG. 6C. Variant MSP828 yielded a high level of on-target activity under these conditions. A-to-G base editing efficiency is shown for on-target and bystander editing.

FIG. 7 shows schematics depicting normal and loss-of-function g6pc function and related outcomes. GSD-Ia (or GSD1a herein) is an autosomal recessive disorder caused by mutations in the g6pc gene. R83C, located in the active site of the enzyme, is the most prevalent pathogenic mutation identified in Caucasian GSD-Ia patients and is associated with inactivation of G6Pase. A loss of G6Pase function can result in life-threatening hypoglycemia, seizures and even death. To mitigate hypoglycemia, patients must maintain strict and frequent adherence to glucose supplementation through day and night, by way of a slow glucose release formula. One missed or delayed dose can result in emergency hypoglycemia. Among many complications, enlarged liver, accumulation of uric acid, lactate, and lipids are common in GSD-Ia patients.

FIG. 8 shows a schematic illustrating that base editors as described herein generate permanent, predicted nucleotide substitutions in an editing window. The R83C mutation introduces a single G>A conversion in the g6pc gene. Adenine base editors (ABEs) enable the programmable conversion of A to G in genomic DNA and thus may be used to correct this mutation. FIG. 8 depicts the utility of ABEs and base editing as described herein. ABE binds to target DNA that is complementary to the guide-RNA and exposes a stretch of single-stranded DNA. The deaminase converts the target adenine into inosine, and the Cas enzyme nicks the opposite strand, which is then repaired, completing the base pair conversion. The direct repair of a point mutation has the potential for restoration of gene function.

FIGS. 9A and 9B provide a depiction of the target nucleotide site, and bystander and PAM nucleotides and a bar graph showing that ABEs used in immortalized HEK293 cells yield a significant rate of precise correction of R83C. Base-editors for A to G conversion in the g6pc gene were optimized for correction of R83C. Shown in FIG. 9A is the target DNA sequence (CCACCAGTATGGACACTGTCCAAAGAGAAT (SEQ ID NO: 17)) and underlying amino acid translation (WWYPCQGFLI; SEQ ID NO: 18) for the GSD-Ia R83C mutation. The target edit is shown by double-underlining, at position 12. The editing window also includes a possible bystander, shown by single-underlining at position 6, and an edit that may result in a synonymous conversion is shown at position 10. For screening, a HEK293 cell line was generated to express the g6pc transgene harboring the R83C mutation and was transfected with base-editor mRNA and gRNA. Allele frequencies were assessed by high-throughput targeted amplicon Next-Generation Sequencing (NGS). Variants 1-5 represent a combination of gRNA and base-editor RNA, engineered for optimized target correction. Variant 5 yielded approximately 60% targeted base-editing efficiency for R83C correction with limited bystander editing (FIG. 9B).

FIG. 10 presents a photographic image and bar graphs demonstrating that 3-week-old homozygous huR83C (Hom huR83C) mice exhibited expected growth impairment and metabolic defects characteristic of GSD-1a. For the experiments, a GSD-Ia mouse that expresses the human G6PC-R83C transgene in place of mouse G6PC was generated to validate base-editing in vivo. The results shown confirmed that mice homozygous for huR83C exhibited postnatal lethality—they were either stillborn or died within 24 hours. On glucose supplementation therapy, the animals survived to at least 3 weeks of age and revealed characteristic pathological signatures of GSD-Ia, with reduced body weight, enlarged livers, significant G6Pase inhibition, and abnormal serum metabolites as compared to littermate controls, a phenotype that is consistent with clinical and published reports.

FIGS. 11A and 11B show dot plots of in vivo correction achieved by the base editors (ABEs) described herein. FIG. 11A illustrates efficient lipid nanoparticle (LNP)-mediated base editing (huG6PC-R83C correction) in livers of adult and newborn heterozygous huR83C mice. To validate base-editing efficiency for R83C correction in vivo, LNP-mediated delivery was first optimized in less fragile transgenic mice heterozygous for huR83C. The schematic in FIG. 6A depicts in vivo workflow for these experiments, with lipid nanoparticle (LNP), or LNP co-formulations of base-editor mRNA and gRNA dosed via IV injection. Given neonatal lethality of the homozygous mice, LNP-dosing was employed via the temporal vein of heterozygous huR83C mice shortly post birth, and activity was compared to that seen in adult heterozygous huR83C mice that had received LNP administered via the tail vein. NGS analysis of whole liver extracts revealed approximately 40% base-editing efficiency in adults and up to ˜60% efficiency in newborns, with a broader range in efficiencies. Bystander editing remained low in adults and newborns. FIG. 11B shows that LNP-mediated R83C correction in livers is associated with survival of newborn homozygous huR83C mice and littermate heterozygous huR83C mice. Briefly, newborn mice homozygous for huR83C were treated with LNP containing guide RNA and mRNA encoding ABE. The treated mice grew normally to 3 weeks of age, without hypoglycemia-induced seizures, in the absence of glucose therapy. The treated homozygous huR83C mice displayed editing efficiencies up to ˜60% in total liver extracts (i.e., ˜60% R83C correction), consistent with littermate controls that were heterozygous for huR83C.

FIGS. 12A and 12B show bar graphs and immunohistochemical staining images demonstrating the base editing as described herein in mice homozygous for huG6PC-R83C restores near-normal metabolic function to reverse GSD-Ia pathology. At 3 weeks, it was validated that the treated homozygous huR83C mice displayed proper metabolic function, with restoration of near-normal serum metabolite markers, including glucose, triglycerides, cholesterol, lactate, and uric acid, as shown by the darkest bars in the graph in FIG. 12A. Moreover, biochemical assays of G6PC activity (as assessed biochemically and via lead-phosphate staining) in LNP-treated homozygous huR83C mice were consistent with that of litter-mate controls. Hepatomegaly, another clinical presentation of GSD-Ia, is caused primarily by excess glycogen and lipid deposition. Immuno-histochemical analysis revealed normal hepatocyte size and lipid deposition in LNP-treated mice (FIG. 12B). The results demonstrate the potential of base-editing to correct the R83C mutation and the metabolic defects associated with GSD-Ia.

FIG. 13 shows a bar graph demonstrating that a single LNP dose administration in homozygous huG6PC-R83C mice maintained euglycemia during a 24-hour fasting challenge via base-editing as described herein.

FIG. 14 shows a Kaplan-Meier survival curves were generated to estimate survival of newborn transgenic mice homozygous for huG6PC-R83C either post base-editing via ABE mRNA or untreated. Newborn mice were genotyped via PCR analysis on genomic tail DNA using the following primers, a universal forward primer (5′-ACCTACTGATGATGCACCTITGATCAATAGAT-3′(SEQ ID NO: 61)), a mouse specific reverse primer (5′-CATCACCCCTCGGGATGGTCTT-3′ (SEQ ID NO: 62)), a human specific reverse primer 1 (5′-CAGCCCAGAATCCCAACCACAAAAT-3′ (SEQ ID NO: 63), and human specific reverse primer 2 (5′-AGACCAGCTCGACTTGGGATGG-3′(SEQ ID NO: 64)). Survival was noted for transgenic mice homozygous for huG6PC-R83C. Untreated mice were either still-born (n=6) or died at 8 hrs (n=6) and 24 hrs (n=1). Administration of 15% glucose injections extended survival to 32 hrs (n=5), 48 hrs (n=2), and 56 hrs (n=2). All ABE-treated mice homozygous for huG6PC-R83C survived to termination of study at 3 wks.

FIG. 15A is a schematic of gRNA fluorescently tagged with Cy5 dye. FIG. 15B is a schematic of gRNA conjugated to NLS fluorescently tagged with Cy5 dye. FIG. 15C shows nuclear staining with Nuc Blue. FIG. 15D shows nuclear staining and ALAS1/sg23 gRNA localization with Cy5. FIG. 15E shows enhanced nuclear localization of NLS-gRNA. FIG. 15F is a graph that shows nuclear localization of gRNA and NLS-gRNA in PXB cells. When conjugated to an NLS, the gRNA was effectively localized to the nucleus. ABE8.8.

FIG. 16 is a model of NLS conjugates bound to saCas9 effectors at the 3′ end.

FIG. 17A provides sequences of exemplary 5% end modified gRNA and exemplary 25% heavy modified saHM03 gRNA. FIG. 17B is a graph that shows results of A-to-G base editing efficiency of exemplary NLS conjugated gRNA relative to end modified gRNA and heavy modified saHM03 gRNA.

### DETAILED DESCRIPTION

Provided herein are methods, compositions and kits to enhance the potency of gRNA for use in CRISPR-Cas systems. The invention provides, in some aspects, methods to produce gRNA conjugated to an NLS sequence (NLS-gRNA) that has increased potency for use in CRISPR-Cas system, increasing frequency of successful editing events. The NLS-gRNA of the present invention can provide better trafficking of the gRNA to the nucleus to protect from cytosolic RNases and increase higher local concentration of gRNA for formation of RNP. NLS-gRNA of the present invention has significantly higher potency as compared to a counterpart gRNA without the NLS sequence and also shows a higher potency as compared to highly modified gRNAs.

gRNAs conjugated to a NLS sequence (NLS-gRNA) have potential numerous advantages that include, for example increased potency. For example, the NLS-gRNA of the present invention provides a significantly higher base editing efficiency relative to its counterpart gRNA without a NLS sequence. Moreover, the NLS-gRNA with end modifications (e.g., comprising 2′OMe modifications at the 3′ end and/or at 5′ end) provides a higher potency as compared to a gRNA that is highly modified (e.g., greater than 40%, greater than 60%, or greater than 88% modified).

Various aspects of the invention are described in detail in the following sections. The use of sections is not meant to limit the invention. Each section can apply to any aspect of the invention. In this application, the use of “or” means “and/or” unless stated otherwise.

Guide RNA (gRNA)

As used herein, guide RNA (gRNA) also refers to guide RNA conjugated to a NLS sequence (NLS-gRNA) unless otherwise noted. A gRNA comprises a polynucleotide sequence complementary to a target sequence. The gRNA hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or 100% complementarity to a target nucleic acid sequence.

In some embodiments, the gRNA is between about 50 nucleotides and 250 nucleotides. In some embodiments, the gRNA is between about 50 nucleotides and 500 nucleotides. In some embodiments, the gRNA is between about 50 nucleotides and 1,000 nucleotides. In some embodiments, the gRNA is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides long. In some embodiments, the gRNA of is between about 50 and 75 nucleotides long. In some embodiments, the gRNA is between about 75 and 100 nucleotides long. In some embodiments, the gRNA is between about 100 and 125 nucleotides long. In some embodiments, the gRNA is between about 125 and 150 nucleotides long. In some embodiments, the gRNA is between about 150 and 175 nucleotides long. In some embodiments, the gRNA is between about 175 and 200 nucleotides long. In some embodiments, the gRNA is between about 200 and 225 nucleotides long. In some embodiments, the gRNA is between about 225 and 250 nucleotides long.

In some embodiments, the gRNA comprises a ligated crRNA and a tracrRNA. Various crRNA and tracrRNA sequences are known in the art, for example those associated with several type II CRISPR-Cas9 systems (e.g., WO2013/176772), Cpf1, SaCas9, Cas12, among others.

A gRNA can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustlX, BLAST, Novoalign, SOAP, Maq, and ELAND.

In some embodiments, a gRNA is designed to target to a unique target sequence within the genome of a cell. In some embodiments, a gRNA is designed to lack a PAM sequence. In some embodiments, a gRNA sequence is designed to have optimal secondary structure using a folding algorithm including mFold or Geneious. In some embodiments, expression of gRNAs may be under an inducible promoter, e.g. hormone inducible, tetracycline or doxycycline inducible, arabinose inducible, or light inducible.

In some embodiments, the gRNA sequence is a “dead crRNAs,” “dead guides,” or “dead guide sequences” that can form a complex with a CRISPR-associated protein and bind specific targets without any substantial nuclease activity.

In some embodiments, the gRNA is chemically modified in the sugar phosphate backbone or base. In some embodiments, the gRNA has one or more of the following modifications 2′O-methyl, 2′-F or locked nucleic acids to improve nuclease resistance or base pairing. In some embodiments, the gRNA may contain modified bases such as 2-thiouridine or N6-methyladenosine.

In some embodiments, the gRNA is conjugated with other oligonucleotides, peptides, proteins, tags, dyes, or polyethylene glycol.

In some embodiments, the gRNA includes an aptamer or riboswitch sequence that binds specific target molecules due to their three-dimensional structure.

In some embodiments, gRNA has two, three, four or five hairpins.

In some embodiments, gRNA includes a transcription termination sequence, which includes a polyT sequences comprising six nucleotides.

Conjugation of gRNA to Nuclear Localization (NLS) Sequence

In one aspect, the present invention provides a gRNA conjugated to a NLS sequence through 3′ end of gRNA. In one aspect, the present invention provides a gRNA conjugated to a NLS sequence through 5′ end of gRNA. In one aspect, the present invention provides a gRNA conjugated to a NLS sequence through an internal site of gRNA.

In embodiments, gRNA is conjugated to NLS via a linker. In embodiments, said linker comprises a chemical moiety (e.g., L) and/or a peptidic moiety (e.g., a peptide spacer).

In embodiments, gRNA is conjugated to NLS directly via a chemical moiety (e.g., L). In embodiments, a chemical moiety (e.g., L) is non-peptidic. In embodiments, a chemical moiety (e.g., L) is covalently attached to both the gRNA and NLS.

In embodiments, gRNA is conjugated to NLS via a peptidic moiety (e.g., a peptide spacer). In embodiments, a peptidic moiety (e.g., a peptide spacer) is covalently attached to both the gRNA and NLS.

In embodiments, gRNA is conjugated to NLS via a linker comprising both a chemical moiety (e.g., L) and a peptidic moiety (e.g., a peptide spacer). In embodiments, such conjugates can have a structure according to Formula (I), where a chemical moiety L (e.g., a non-peptidic chemical moiety) is covalently attached to gRNA and a peptide spacer, and wherein the peptide spacer is covalently attached to NLS.

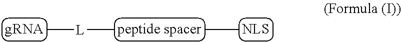

In some embodiments, the N-terminus of NLS sequence is conjugated to the 3′ end of the gRNA via a linker comprising both a chemical moiety (e.g., L) and a peptide moiety (e.g., a peptide spacer). In some embodiments, the C-terminus of NLS sequence is conjugated to the 5′ end of the gRNA via a linker comprising both a chemical moiety (e.g., L) and a peptide moiety (e.g., a peptide spacer). In some embodiments, an internal amino acid in the NLS sequence is conjugated to the 3′ end of the gRNA via a linker comprising both a chemical moiety (e.g., L) and a peptide moiety (e.g., a peptide spacer). In some embodiments, an internal amino acid in the NLS sequence is conjugated to the 5′ end of the gRNA via a linker comprising both a chemical moiety (e.g., L) and a peptide moiety (e.g., a peptide spacer). In some embodiments, an internal amino acid in the NLS sequence is conjugated to an internal nucleotide of the gRNA via a linker comprising both a chemical moiety (e.g., L) and a peptide moiety (e.g., a peptide spacer).

In embodiments, gRNA is conjugated to NLS via a chemical moiety (e.g., L) covalently attached to the C-terminus of the peptide spacer or the NLS amino acid sequence.

In embodiments, gRNA is conjugated to NLS via a chemical moiety (e.g., L) covalently attached to the N-terminus of the peptide spacer or the NLS amino acid sequence.

In embodiments, gRNA is conjugated to the peptide spacer or the NLS via a chemical moiety (e.g., L) covalently attached to the 3′ end of the gRNA.

In embodiments, gRNA is conjugated to the peptide spacer or the NLS via a chemical moiety (e.g., L) covalently attached to the 5′ end of the gRNA.

In embodiments, a chemical moiety (e.g., L) is covalently attached to a thiol-containing residue (e.g., a cysteine residue) of the peptide spacer or the NLS.

In embodiments, a chemical moiety (e.g., L) is covalently attached to a selenium-containing residue (e.g., a selenocysteine residue) of the peptide spacer or the NLS.

In embodiments, a chemical moiety (e.g., L) is covalently attached to an amino-containing residue (e.g., a lysine residue) of the peptide spacer or the NLS.

In embodiments, a chemical moiety (e.g., L) is covalently attached to a phenol-containing residue (e.g., a tyrosine residue) of the peptide spacer or the NLS.

In embodiments, amino acid residues used for formation of a linker (e.g., a thiol-, selenium-, amino-, or phenol-containing residue as described herein) comprise chemical modifications.

In some embodiments, a gRNA is conjugated to a NLS via reductive amination. In some embodiments, a gRNA is conjugated to a NLS native chemical ligation. a gRNA is conjugated to a NLS via thiolene click.

Exemplary chemistries useful for preparing linkers are described herein. Chemical moieties described herein may further including substructures L¹ and/or L², where L¹ and L² are each independently an optionally substituted group that is C₁₋₁₂ alkylene or C₂₋₁₂ heteroalkylene. In embodiments, L¹ and L² comprise an oxo (═O) substituent (e.g., 1 or 2 oxo substituents).

### Maleimide-Thiol/Maleimide-Selenol Adduct

In embodiments, a chemical moiety (e.g., L) comprises a maleimide-thiol adduct.

In embodiments, gRNA is conjugated to NLS using an addition reaction between a maleimide group and a thiol group or a thiol-ene click reaction.

In embodiments, a maleimide-thiol adduct containing moiety is formed from a gRNA comprising a thiol group, and a NLS (or a peptide spacer) comprising a maleimide group. In embodiments, a maleimide-thiol adduct containing moiety is formed from a gRNA comprising a maleimide group, and a NLS (or a peptide spacer) comprising a thiol group.

In embodiments, a chemical moiety (e.g., L) comprises a maleimide-selenol adduct. In embodiments, gRNA is conjugated to NLS using an addition reaction between a maleimide group and a selenol group. In embodiments, a maleimide-selenol adduct containing moiety is formed from a gRNA comprising a selenol group, and a NLS (or a peptide spacer) comprising a maleimide group. In embodiments, a maleimide-selenol adduct containing moiety is formed from a gRNA comprising a maleimide group, and a NLS (or a peptide spacer) comprising a selenol group.

In embodiments, a chemical moiety (e.g., L) comprises

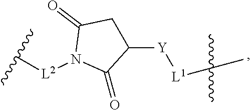

wherein Y is S or Se.

In embodiments, Y is S. In embodiments, a chemical moiety (e.g., L) comprises

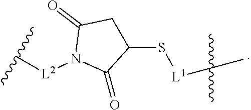

In embodiments, the

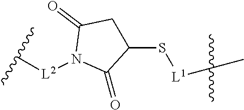

moiety is formed from a gRNA comprising a thiol group, and a NLS (or a peptide spacer) comprising a maleimide group. In embodiments, the maleimide-thiol adduct containing moiety is formed from a gRNA comprising a maleimide group, and a NLS (or a peptide spacer) comprising a thiol group.

In embodiments, Y is Se. In embodiments, a chemical moiety (e.g., L) comprises

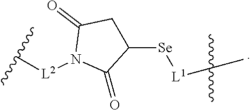

In embodiments, the

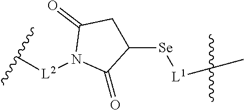

moiety is formed from a gRNA comprising a selenol group, and a NLS (or a peptide spacer) comprising a maleimide group. In embodiments, the maleimide-selenol adduct containing moiety is formed from a gRNA comprising a maleimide group, and a NLS (or a peptide spacer) comprising a selenol group.

In embodiments, a chemical moiety L has the following structure (A), where Y is S or Se,

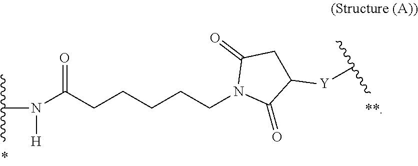

In embodiments, Y is S. In embodiments, * represents covalent attachment to gRNA. In embodiments, ** represents covalent attachment to a peptide spacer or NLS. In embodiments, ** represents covalent attachment to a peptide spacer.

### Thioether/Selenoether

In embodiments, a chemical moiety (e.g., L) comprises a thioether group.

In embodiments, gRNA is conjugated to NLS using a conjugation reaction between an iodoacetamide group and a thiol group.

In embodiments, a thioether-containing moiety is formed from a gRNA comprising a thiol group, and a NLS (or a peptide spacer) comprising an iodoacetamide group. In embodiments, a thioether-containing moiety is formed from a gRNA comprising an iodoacetamide group, and a NLS (or a peptide spacer) comprising a thiol group.

In embodiments, a chemical moiety (e.g., L) comprises a selenoether moiety. In embodiments, gRNA is conjugated to NLS using a conjugation reaction between an iodoacetamide group and a selenol group. In embodiments, a selenoether-containing moiety is formed from a gRNA comprising a selenol group, and a NLS (or a peptide spacer) comprising an iodoacetamide group. In embodiments, a selenoether-containing moiety is formed from a gRNA comprising an iodoacetamide group, and a NLS (or a peptide spacer) comprising a selenol group.

In embodiments, a chemical moiety (e.g., L) comprises

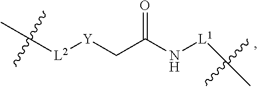

wherein Y is S or Se.

In embodiments, Y is S. In embodiments, a chemical moiety (e.g., L) comprises

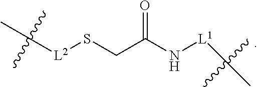

In embodiments, the

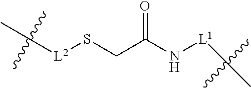

moiety is formed from a gRNA comprising a thiol group, and a NLS (or a peptide spacer) comprising an iodoacetamide group. In embodiments, the

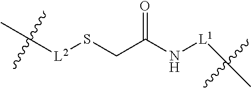

moiety is formed from a gRNA comprising an iodoacetamide group, and a NLS (or a peptide spacer) comprising a thiol group.

In embodiments, Y is Se. In embodiments, a chemical moiety (e.g., L) comprises

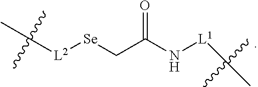

In embodiments, the

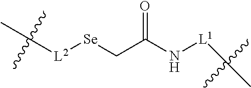

moiety is formed from a gRNA comprising a selenol group, and a NLS (or a peptide spacer) comprising an iodoacetamide group. In embodiments, the

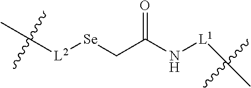

moiety is formed from a gRNA comprising an iodoacetamide group, and a NLS (or a peptide spacer) comprising a selenol group.

### Disulfide (Thiol-Disulfide Exchange Chemistry)

In embodiments, a chemical moiety (e.g., L) comprises a disulfide group. In embodiments, gRNA is conjugated to NLS using a thiol-disulfide exchange reaction between a disulfide-containing group and a thiol group.

In embodiments, the disulfide-containing moiety is formed from a gRNA comprising a thiol group, and a NLS (or a peptide spacer) comprising a disulfide group. In embodiments, the disulfide-containing moiety is formed from a gRNA comprising a disulfide group, and a NLS (or a peptide spacer) comprising a thiol group.

In embodiments, a chemical moiety (e.g., L) comprises

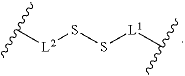

In embodiments, the

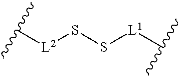

moiety is formed from a gRNA comprising a thiol group, and a NLS (or a peptide spacer) comprising a disulfide group. In embodiments, the

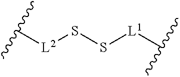

moiety is formed from a gRNA comprising a disulfide group, and a NLS (or a peptide spacer) comprising a thiol group.

### Oxadiazole Thioether

In embodiments, a chemical moiety (e.g., L) comprises an oxadiazole thioether group. In embodiments, gRNA is conjugated to NLS using a reaction between a thiol group and a sulfonyloxadiazole group.

In embodiments, an oxadiazole thioether-containing moiety is formed from a gRNA comprising a sulfonyloxadiazole group, and a NLS (or a peptide spacer) comprising a thiol group. In embodiments, an oxadiazole thioether-containing moiety is formed from a gRNA comprising a thiol group, and a NLS (or a peptide spacer) comprising a sulfonyloxadiazole group.

In embodiments, a chemical moiety (e.g., L) comprises

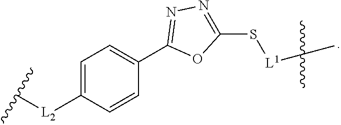

In embodiments, the

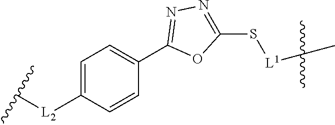

moiety is formed from a gRNA comprising a sulfonyloxadiazole group, and a NLS (or a peptide spacer) comprising a thiol group. In embodiments, the

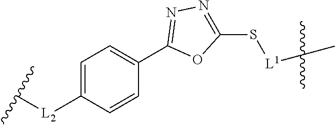

moiety is formed from a gRNA comprising a thiol group, and a NLS (or a peptide spacer) comprising a sulfonyloxadiazole group.

### Urea/Thiourea/Dithiocarbamate (Iso(Thio)Cyanate Chemistry)

In embodiments, a chemical moiety (e.g., L) comprises a urea group. In embodiments, gRNA is conjugated to NLS using a reaction between an amino (e.g., primary amine) group and an isocyanate group.

In embodiments, a urea-containing moiety is formed from a gRNA comprising an amino (e.g., primary amine) group, and a NLS (or a peptide spacer) comprising an isocyanate group. In embodiments, a urea-containing moiety is formed from a gRNA comprising an isocyanate group, and a NLS (or a peptide spacer) comprising an amino (e.g., primary amine) group.

In embodiments, a chemical moiety (e.g., L) comprises a thiourea group. In embodiments, gRNA is conjugated to NLS using a reaction between an amino (e.g., primary amine) group and an isothiocyanate group. In embodiments, a thiourea-containing moiety is formed from a gRNA comprising an amino (e.g., primary amine) group, and a NLS (or a peptide spacer) comprising an isothiocyanate group. In embodiments, a thiourea-containing moiety is formed from a gRNA comprising an isothiocyanate group, and a NLS (or a peptide spacer) comprising an amino (e.g., primary amine) group.

In embodiments, a chemical moiety (e.g., L) comprises

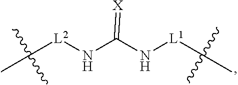

wherein X is S or O.

In embodiments, X is O. In embodiments, a chemical moiety (e.g., L) comprises

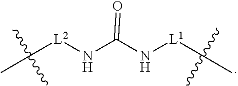

In embodiments, the

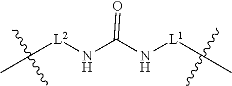

moiety is formed from a gRNA comprising an amino (e.g., primary amine) group, and a NLS (or a peptide spacer) comprising an isocyanate group. In embodiments, the

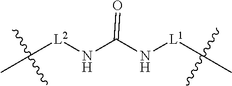

moiety is formed from a gRNA comprising an isocyanate group, and a NLS (or a peptide spacer) comprising an amino (e.g., primary amine) group.

In embodiments, X is S. In embodiments, a chemical moiety (e.g., L) comprises

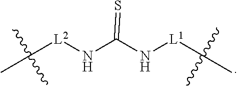

In embodiments, the

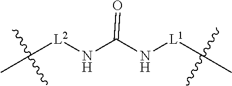

moiety is formed from a gRNA comprising an amino (e.g., primary amine) group, and a NLS (or a peptide spacer) comprising an isothiocyanate group. In embodiments, the

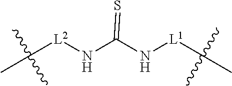

moiety is formed from a gRNA comprising an isothiocyanate group, and a NLS (or a peptide spacer) comprising an amino (e.g., primary amine) group.

In embodiments, a chemical moiety (e.g., L) comprises a dithiocarbamate group. In embodiments, gRNA is conjugated to NLS using a reaction between a thiol group and an isothiocyanate group.

In embodiments, a dithiocarbamate-containing moiety is formed from a gRNA comprising a thiol group, and a NLS (or a peptide spacer) comprising an isothiocyanate group. In embodiments, a dithiocarbamate-containing moiety is formed from a gRNA comprising an isothiocyanate group, and a NLS (or a peptide spacer) comprising a thiol group.

In embodiments, a chemical moiety (e.g., L) comprises

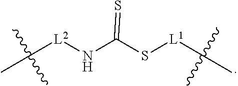

In embodiments, the

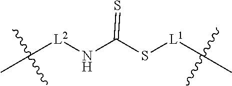

moiety is formed from a gRNA comprising a thiol group, and a NLS (or a peptide spacer) comprising an isothiocyanate group. In embodiments, the

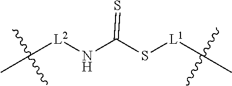

moiety is formed from a gRNA comprising an isothiocyanate group, and a NLS (or a peptide spacer) comprising a thiol group.

### Diazenylphenol

In embodiments, a chemical moiety (e.g., L) comprises a diazenylphenol group. In embodiments, gRNA is conjugated to NLS using a reaction between a phenol group and a diazonium group.

In embodiments, a diazenylphenol-containing moiety is formed from a gRNA comprising a phenol group, and a NLS (or a peptide spacer) comprising a diazonium group. In embodiments, a diazenylphenol-containing moiety is formed from a gRNA comprising a diazonium group, and a NLS (or a peptide spacer) comprising a phenol group.

In embodiments, In embodiments, a chemical moiety (e.g., L) comprises

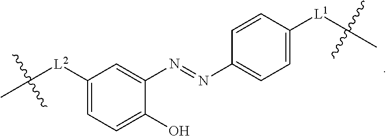

In embodiments, the

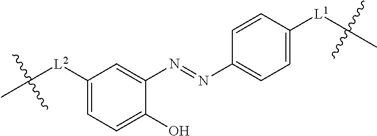

moiety is formed from a gRNA comprising a phenol group, and a NLS (or a peptide spacer) comprising a diazonium group. In embodiments, the

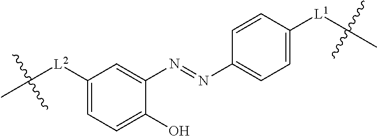

moiety is formed from a gRNA comprising a diazonium group, and a NLS (or a peptide spacer) comprising a phenol group.

### Triazolidinedionylphenol

In embodiments, a chemical moiety (e.g., L) comprises a triazolidinedionylphenol group. In embodiments, gRNA is conjugated to NLS using a reaction between a phenol group and a cyclic diazodicarboxamide group.

In embodiments, a triazolidinedionylphenol-containing moiety is formed from a gRNA comprising a phenol group, and a NLS (or a peptide spacer) comprising a cyclic diazodicarboxamide group. In embodiments, a triazolidinedionylphenol-containing moiety is formed from a gRNA comprising a cyclic diazodicarboxamide group, and a NLS (or a peptide spacer) comprising a phenol group.

In embodiments, a chemical moiety (e.g., L) comprises

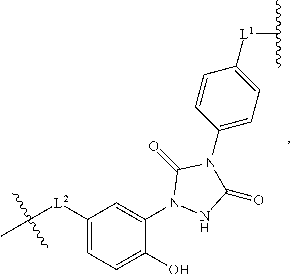

In embodiments, the

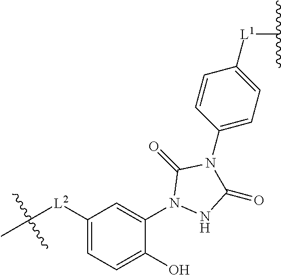

moiety is formed from a gRNA comprising a phenol group, and a NLS (or a peptide spacer) comprising a cyclic diazodicarboxamide group. In embodiments, the

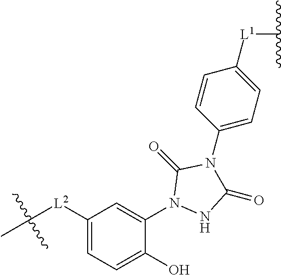

moiety is formed from a gRNA comprising a cyclic diazodicarboxamide group, and a NLS (or a peptide spacer) comprising a phenol group.

### Triazole (Click Chemistry)

In embodiments, a chemical moiety (e.g., L) comprises a triazole group. In embodiments, gRNA is conjugated to NLS using a 1,3-dipolar cycloaddition between an alkyne group and an azide group.

In embodiments, a triazole-containing moiety is formed from a gRNA comprising an alkyne group and a NLS (or a peptide spacer) comprising an azide group. In embodiments, a triazole-containing moiety is formed from a gRNA comprising an azide group and a NLS (or a peptide spacer) comprising an alkyne group. In embodiments, a 1,3-dipolar cycloaddition is copper-catalyzed cycloaddition. In embodiments, a 1,3-dipolar cycloaddition is strain-promoted cycloaddition.

In embodiments, a chemical moiety (e.g., L) comprises

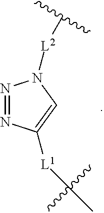

In embodiments, the

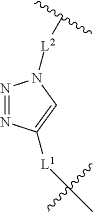

moiety is formed from a gRNA comprising an alkyne group and a NLS (or a peptide spacer) comprising an azide group. In embodiments, the

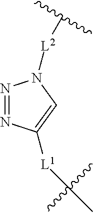

moiety is formed from a gRNA comprising an azide group and a NLS (or a peptide spacer) comprising an alkyne group.

In embodiments, a chemical moiety (e.g., L) comprises

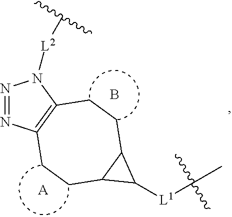

wherein each of ring A and ring B are optionally substituted aryl groups. In embodiments, ring A is present. In embodiments, ring A is not present. In embodiments, ring B is present. In embodiments, ring B is not present. In embodiments, both ring A and ring B are present. In embodiments, both ring A and ring B are not present. In embodiments, the

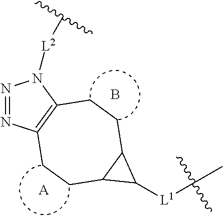

moiety is formed from a gRNA comprising an alkyne group and a NLS (or a peptide spacer) comprising an azide group. In embodiments, the

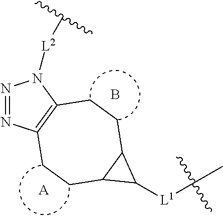

moiety is formed from a gRNA comprising an azide group and a NLS (or a peptide spacer) comprising an alkyne group.

In embodiments, a chemical moiety (e.g., L) comprises

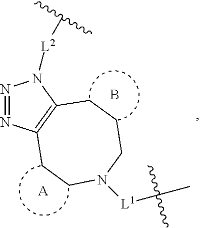

wherein each of ring A and ring B are optionally substituted aryl groups. In embodiments, ring A is present. In embodiments, ring A is not present. In embodiments, ring B is present. In embodiments, ring B is not present. In embodiments, both ring A and ring B are present. In embodiments, both ring A and ring B are not present. In embodiments, the

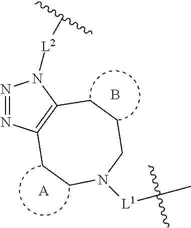

moiety is formed from a gRNA comprising an alkyne group and a NLS (or a peptide spacer) comprising an azide group. In embodiments, the

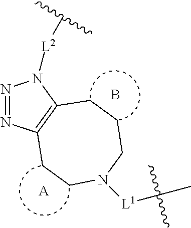

moiety is formed from a gRNA comprising an azide group and a NLS (or a peptide spacer) comprising an alkyne group.

### Diazanorcaradiene

In embodiments, a chemical moiety (e.g., L) comprises a diazanorcaradiene group. In embodiments, gRNA is conjugated to NLS using a Diels-Alder reaction between a cyclopropene group and a tetrazine group.

In embodiments, a diazanorcaradiene-containing moiety is formed from a gRNA comprising a cyclopropene group and a NLS (or a peptide spacer) comprising a tetrazine group. In embodiments, a diazanorcaradiene-containing moiety is formed from a gRNA comprising a tetrazine group and a NLS (or a peptide spacer) comprising a cyclopropene group.

In embodiments, a chemical moiety (e.g., L) comprises

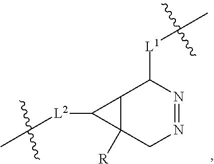

wherein R is a C₁₋₆ alkyl. In embodiments, the

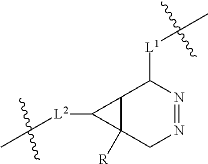

moiety is formed from a gRNA comprising a cyclopropene group and a NLS (or a peptide spacer) comprising a tetrazine group. In embodiments, the

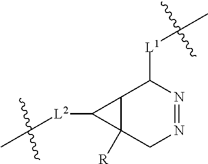

moiety is formed from a gRNA comprising a tetrazine group and a NLS (or a peptide spacer) comprising a cyclopropene group.

### Amide/Sulfonamide

In embodiments, a chemical moiety (e.g., L) comprises an amide group. In embodiments, gRNA is conjugated to NLS using a conjugation reaction between a carboxyl group and an amino group (e.g., primary amine).

In embodiments, an amide-containing moiety is formed from a gRNA comprising a carboxyl group and a NLS (or a peptide spacer) comprising an amino (e.g., primary amine) group. In embodiments, an amide-containing moiety is formed from a gRNA comprising an amino group (e.g., primary amine) and a NLS (or a peptide spacer) comprising a carboxyl group. In embodiments, a carboxyl group is an activated carboxyl group. In embodiments, the carboxyl group is activated by carbodiimides such as 1-ethyl-3-(3-dimethyl-aminopropyl) carbodiimide (EDC) or dicyclohexylcarbodiimide (DCC). In embodiments, the carboxyl group is activated by N-hydroxysuccinimide (NHS) derivatives (e.g., sulfo-NHS).

In embodiments, a chemical moiety (e.g., L) comprises

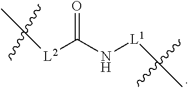

In embodiments, the

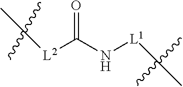

moiety is formed from a gRNA comprising a carboxyl group and a NLS (or a peptide spacer) comprising an amino (e.g., primary amine) group. In embodiments, the

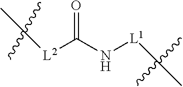

moiety is formed from a gRNA comprising an amino group (e.g., primary amine) and a NLS (or a peptide spacer) comprising a carboxyl group.

In embodiments, a chemical moiety (e.g., L) comprises a sulfonamide group. In embodiments, gRNA is conjugated to NLS using a conjugation reaction between a sulfonyl group and an amino (e.g., primary amine) group. In embodiments, a sulfonamide-containing moiety is formed from a gRNA comprising a sulfonyl group and a NLS (or a peptide spacer) comprising an amino (e.g., primary amine) group. In embodiments, an amide-containing moiety is formed from a gRNA comprising an amino (e.g., primary amine) group and a NLS (or a peptide spacer) comprising a sulfonyl group.

In embodiments, a chemical moiety (e.g., L) comprises

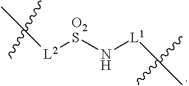

In embodiments, the

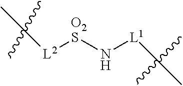

moiety is formed from a gRNA comprising a sulfonyl group and a NLS (or a peptide spacer) comprising an amino (e.g., primary amine) group. In embodiments, the

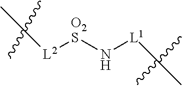

moiety is formed from a gRNA comprising an amino (e.g., primary amine) group and a NLS (or a peptide spacer) comprising a sulfonyl group.

### Amine (Glutaraldehyde Chemistry)

In embodiments, a chemical moiety (e.g., L) comprises an amino group. In embodiments, gRNA is conjugated to NLS using a conjugation reaction between an amino group (e.g., primary amine) and an aldehyde group followed by a reduction reaction to form an amine-containing moiety.

In embodiments, an amine-containing moiety is formed from a gRNA comprising an amino group (e.g., primary amine), and a NLS (or a peptide spacer) comprising an aldehyde group. In embodiments, an amine-containing moiety is formed from a gRNA comprising an aldehyde group, and a NLS (or a peptide spacer) comprising an amino group (e.g., primary amine).

In embodiments, an amine-containing moiety is formed from a bifunctional cross-linking reagent (e.g., a dialdehyde such as glutaraldehyde). In embodiments, an amine-containing moiety is formed from a gRNA comprising an amino group (e.g., primary amine), a NLS (or a peptide spacer) comprising an amino group (e.g., primary amine), and a dialdehyde (e.g., glutaraldehyde). In embodiments, an amine-containing moiety is formed from a gRNA comprising an aldehyde group, a NLS (or a peptide spacer) comprising an aldehyde group, and a diaminoalkane.

In embodiments, a chemical moiety (e.g., L) comprises

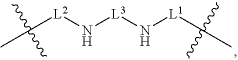

wherein L³ is a C₁₋₆ alkyl. In embodiments, the

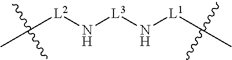

moiety is formed from a gRNA comprising an amino group (e.g., primary amine), a NLS (or a peptide spacer) comprising an amino group (e.g., primary amine), and a dialdehyde (e.g., glutaraldehyde). In embodiments, the

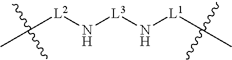

moiety is formed from a gRNA comprising an aldehyde group, a NLS (or a peptide spacer) comprising an aldehyde group, and a diaminoalkane.

In embodiments, a chemical moiety (e.g., L) comprises an amino group. In embodiments, gRNA is conjugated to NLS using a conjugation reaction between an amino (e.g., a primary amine) group and a tresyl (2,2,2-Trifluoroethanesulfonyl) group. In embodiments, an amine moiety is formed from a gRNA comprising an amino (e.g., a primary amine) group, and a NLS (or a peptide spacer) comprising a tresyl (2,2,2-Trifluoroethanesulfonyl) group. In embodiments, an amine-containing moiety is formed from a gRNA comprising a tresyl (2,2,2-Trifluoroethanesulfonyl) group and a NLS (or a peptide spacer) comprising an amino (e.g., a primary amine) group.

In embodiments, a chemical moiety (e.g., L) comprises

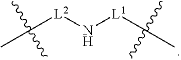

In embodiments, the

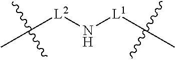

moiety is formed from a gRNA comprising an amino (e.g., a primary amine) group, and a NLS (or a peptide spacer) comprising a tresyl (2,2,2-Trifluoroethanesulfonyl) group. In embodiments, the

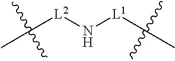

moiety is formed from a gRNA comprising a tresyl (2,2,2-Trifluoroethanesulfonyl) group and a NLS (or a peptide spacer) comprising an amino (e.g., a primary amine) group.

In some embodiments, the NLS-gRNA comprises a crRNA. In some embodiments, the NLS-gRNA comprises a tracrRNA. In some embodiments, the NLS-gRNA comprises a crRNA and a NLS-gRNA.

In some embodiments, a linear guide RNA is first synthesized. In this approach, two or more separate RNAs are ligated together. In some embodiments, a first RNA comprises a trans-activating RNA (tracrRNA), and a second RNA comprises a clustered regularly interspersed short palindromic repeats (CRISPR) RNA (crRNA).

In some embodiments, the RNA comprising the tracrRNA sequences are synthesized such that a portion of the tracrRNA contains a phosphate at the 5′-terminus. Two forms of ligation are possible with this approach, both of which are found within the stem loop region. The first form of ligation occurs within the terminal loop of the hairpin, which is a natural site of T4 RNA Ligase 1. The second form of ligation occurs within the duplex which is a natural of T4 RNA Ligase 2 and DNA ligases. One of the advantages of this form of ligation is that fragment impurities are readily removable because of the marked differences in elution time between the fused gRNA and the fragment impurities.

### Chemically Modified NLS-rRNA

In some embodiments, the first end of the guide RNA and/or the second end of the guide RNA comprises a chemical modification to its backbone or to one or more of its bases. For example, chemically modified RNA can comprise chemical synthesis can be used to install highly modified monomers including modified sugars, bases, backbones or functional groups that do not resemble natural nucleotides.

Accordingly, in some embodiments, the first end of the guide RNA and/or the second end of the guide RNA comprises a modified base. In some embodiments, the modified RNA include one or more of the following 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′-O-Methyl modifications can also be used with the methods described herein. For example, the following RNA comprising one or more of the following 2′OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the first end of the guide RNA and/or second end of the guide RNA comprises one or more of the following modifications: phosphorothioates, 2′O-methyl, 2′ fluoro (2′F), DNA.

In some embodiments, the first end of the guide RNA and/or the second end of the guide RNA comprises 2′OMe modifications at the 3′ and 5′-ends.

In some embodiments, the first end of the guide RNA and/or second end of the guide RNA comprises one or more of the following modifications: 2′-O-2-Methoxyethyl (MOE), locked nucleic acids, bridged nucleic acids, unlocked nucleic acids, peptide nucleic acids, morpholino nucleic acids.

In some embodiments, the first end of the guide RNA and/or second end of the guide RNA comprises one or more of the following base modifications: 2,6-diaminopurine, 2-aminopurine, pseudouracil, NI-methyl-pseudouracil, 5′ methyl cytosine, 2′pyrimidinone (zebularine), thymine.

Other modified bases include for example, 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Desoxy-r(A,C,G,U), 3′-O-Methyl-r(A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the first end of the guide RNA and/or second end of the guide RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase or cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the nucleobase editors provided herein comprise one or more features that improve base editing activity. For example, any of the nucleobase editors provided herein may comprise a Cas9 domain that has reduced nuclease activity. In some embodiments, any of the nucleobase editors provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand opposite the targeted nucleobase. Mutation of the catalytic residue (e.g., D10 to A10) prevents cleavage of the edited (e.g., deaminated) strand containing the targeted residue (e.g., A or C). Such Cas9 variants can generate a single-strand DNA break (nick) at a specific location based on the gRNA-defined target sequence, leading to repair of the non-edited strand, ultimately resulting in a nucleobase change on the non-edited strand.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain can comprise an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Fusion Proteins with Internal Insertions

Provided herein are fusion proteins comprising a heterologous polypeptide fused to a nucleic acid programmable nucleic acid binding protein, for example, a nucleic acid programmable DNA binding protein (napDNAbp). A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp.

In some embodiments, the heterologous polypeptide is inserted at an internal location of the napDNAbp. In some embodiments, the heterologous polypeptide is a deaminase (e.g., adenosine deaminase) or a functional fragment thereof. For example, a fusion protein can comprise a deaminase (e.g., adenosine deaminase) flanked by an N-terminal fragment and a C-terminal fragment of a Cas9 polypeptide. The deaminase in a fusion protein can be an adenosine deaminase. In some embodiments, the adenosine deaminase is a TadA (e.g., TadA*7.10 or a variant thereof).

In some embodiments, the fusion protein comprises the structure:

NH2-[N-terminal fragment of a napDNAbp]-[deaminase]-[C-terminal fragment of a napDNAbp]-COOH;

NH2-[N-terminal fragment of a Cas9]-[adenosine deaminase]-[C-terminal fragment of a Cas9]-COOH;
wherein each instance of “]-[” is an optional linker.

The deaminase can be a circular permutant deaminase. For example, the deaminase can be a circular permutant adenosine deaminase. In some embodiments, the deaminase is a circular permutant TadA, circularly permutated at amino acid residue 116 as numbered in the TadA reference sequence. In some embodiments, the deaminase is a circular permutant TadA, circularly permutated at amino acid residue 136 as numbered in the TadA reference sequence. In some embodiments, the deaminase is a circular permutant TadA, circularly permutated at amino acid residue 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one deaminase. In some embodiments, the fusion protein comprises two deaminases. The two or more deaminases in a fusion protein can be an adenosine deaminase, cytidine deaminase, or a combination thereof. The two or more deaminases can be homodimers. The two or more deaminases can be heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants thereof.

Fusion proteins comprising a heterologous catalytic domain flanked by N- and C-terminal fragments of a Cas9 polypeptide are also useful for base editing in the methods as described herein. Fusion proteins comprising Cas9 and one or more deaminase domains, e.g., adenosine deaminase, or comprising an adenosine deaminase domain flanked by Cas9 sequences are also useful for highly specific and efficient base editing of target sequences. In an embodiment, a chimeric Cas9 fusion protein contains a heterologous catalytic domain (e.g., adenosine deaminase, cytidine deaminase, or adenosine deaminase and cytidine deaminase) inserted within a Cas9 polypeptide. In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:

NH2-[Cas9(adenosine deaminase)]-[cytidine deaminase]-COOH;

NH2-[cytidine deaminase]-[Cas9(adenosine deaminase)]-COOH;

NH2-[Cas9(cytidine deaminase)]-[adenosine deaminase]-COOH; or

NH2-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA variant. In some embodiments, a TadA variant is fused within Cas9 and a cytidine deaminase is fused to the C-terminus. In some embodiments, a TadA variant is fused within Cas9 and a cytidine deaminase fused to the N-terminus. In some embodiments, a cytidine deaminase is fused within Cas9 and a TadA variant is fused to the C-terminus. In some embodiments, a cytidine deaminase is fused within Cas9 and a TadA variant fused to the N-terminus. Exemplary structures of a fusion protein with a TadA variant and a cytidine deaminase and a Cas9 are provided as follows:

NH2-[Cas9(TadA variant)]-[cytidine deaminase]-COOH;

NH2-[cytidine deaminase]-[Cas9(TadA variant)]-COOH;

NH2-[Cas9(cytidine deaminase)]-[TadA variant]-COOH; or

NH2-[TadA variant]-[Cas9(cytidine deaminase)]-COOH.

In some embodiments, the “-” used in the general architecture above indicates the presence of an optional linker.

In other embodiments, the fusion protein contains a nuclear localization signal (e.g., a bipartite nuclear localization signal). In other embodiments, the amino acid sequence of the nuclear localization signal is MAPKKKRKVGIHGVPAA (SEQ ID NO: 4). In other embodiments of the above aspects, the nuclear localization signal is encoded by the following sequence:
ATGGCCCCAAAGAAGAAGCGGAAGGTCGGTATCCACGGAGTCCCAGCAG CC (SEQ ID NO: 5). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b.

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

Gene Editing Using gRNA

The NLS-gRNA described herein can be used with a suitable gene editing system for targeted gene editing which can result in a gene silencing event, or an alteration of the expression (e.g., an increase or a decrease) in the expression of a desired target gene. Accordingly, in some embodiments, the NLS-gRNA described herein can be used in a method for targeted transcription activation, targeted transcription repression, targeted epigenome modification, or targeted genome modification, the method comprising introducing into a eukaryotic cell: (a) a NLS-gRNA as defined herein; (b) at least one CRISPR/Cas protein or a nucleic acid encoding the at least one CRISPR/Cas protein; wherein interactions between (a) and (b) and a target sequence in chromosomal DNA leads to targeted transcription activation, targeted transcription repression, targeted epigenome modification, or targeted genome modification.

In some embodiments, the NLS-gRNA described herein can be used in a gene editing system comprising: the NLS-gRNA described herein, wherein the RNA guide comprises a direct repeat sequence and a spacer sequence capable of hybridizing to a target nucleic acid; gene editing protein, and wherein the gene editing enzyme is capable of binding to the RNA guide and of causing a break in the target nucleic acid sequence complementary to the RNA guide.

In some embodiments, the NLS-gRNA described herein can be used in a gene editing system comprising: the NLS-gRNA described herein, wherein the RNA guide comprises a direct repeat sequence and a spacer sequence capable of hybridizing to a target nucleic acid; and a gene editing protein; wherein the gene editing protein is fused to a deaminase, and wherein the gene editing protein fusion is capable of binding to the RNA guide and of editing the target nucleic acid sequence complementary to the RNA guide.

In some embodiments, the invention provides a method of altering expression of a target nucleic acid in a eukaryotic cell comprising: contacting the cell with a gene editing protein, and the NLS-gRNA described herein, wherein the NLS-gRNA comprises a direct repeat sequence and a spacer sequence capable of hybridizing to the target nucleic acid, and wherein the gene editing protein is capable of binding to the NLS-gRNA and of causing a break in the target nucleic acid sequence complementary to the NLS-gRNA.

In some embodiments, the invention provides a method of altering expression of a target nucleic acid in a eukaryotic cell comprising: contacting the cell with a gene editing protein, and the synthetic NLS-gRNA described herein, wherein the NLS-gRNA comprises a direct repeat sequence and a spacer sequence capable of hybridizing to the target nucleic acid, and wherein the gene editing protein is capable of binding to the NLS-gRNA and editing the target nucleic acid sequence complementary to the NLS-gRNA.

In some embodiments, the invention provides a method of modifying a target nucleic acid in a eukaryotic cell comprising: contacting the cell with a gene editing protein, and the NLS-gRNA described herein, wherein the NLS-gRNA comprises a direct repeat sequence and a spacer sequence capable of hybridizing to the target nucleic acid, and wherein the gene editing protein is capable of binding to the NLS-gRNA and editing the target nucleic acid sequence complementary to the NLS-gRNA.

In some embodiments, the gene editing method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the gene editing method or system comprises a fusion protein with enzymes that can edit DNA sequences by chemically modifying nucleotide bases, including deaminase enzymes that can modify adenosine or cytosine bases and function as site-specific base editors. For example, APOBEC1 cytidine deaminase, which usually uses RNA as a substrate, can be targeted to single-stranded and double-stranded DNA when it is fused to Cas9, converting cytidine to uridine directly, and ADAR enzymes deaminate adenosine to inosine. Thus, ‘base editing’ using deaminases enables programmable conversion of one target DNA base into another. Various base editors are known in the art and can be used in the method and systems described herein. Exemplary base editors are described in, for example, Rees and Liu Nature Review Genetics, 2018, 19(12): 770-788, the contents of which are incorporated herein.

In some embodiments, base editing results in the introduction of stop codons to silence genes. In some embodiments, base editing results in altered protein function by altering amino acid sequences.

In some embodiments, the NLS-gRNA described herein can be used in a gene editing method or system to modulate transcription of target DNA. In some embodiments, the NLS-gRNA can be used in a gene editing method or system to modulate the expression of a target non-coding RNA, including tRNA, rRNA, snoRNA, siRNA, miRNA, and long ncRNA.

In some embodiments, the NLS-gRNA described herein is used for targeted engineering of chromatin loop structures using a suitable gene editing system. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, the NLS-gRNA described herein is used in conjunction with a gene editing system for correction of pathogenic mutations by insertion of beneficial clinical variants or suppressor mutations.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase.

In some embodiments, a base editor described herein comprises a fusion protein comprising an adenosine deaminase domain (e.g., adenosine deaminase variant domain). In some embodiments, an adenosine deaminase variant domain contains a combination of alterations in a TadA*7.10 amino acid sequence, where the combinations are V82G, Y147T/D, Q154S, and one or more of L36H, I76Y, F149Y, N157K, and D167N. In some embodiments, the combinations of alterations in a TadA*7.10 amino acid sequence are V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N or a corresponding alteration in another adenosine deaminase. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA).

In some embodiments, the nucleobase editors provided herein can be made by fusing together one or more protein domains, thereby generating a fusion protein. In certain embodiments, the fusion proteins provided herein comprise one or more features that improve the base editing activity (e.g., efficiency, selectivity, and specificity) of the fusion proteins. For example, the fusion proteins provided herein can comprise a Cas9 domain that has reduced nuclease activity. In some embodiments, the fusion proteins provided herein can have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2). In another embodiment, an adenosine deaminase incorporated into a base editor comprises all or a portion of adenosine deaminase acting on tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

### Adenosine Deaminases

In some embodiments, the fusion proteins as described herein comprise one or more adenosine deaminase domains. In some embodiments, the adenosine deaminases provided herein are capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenine deaminase is a naturally-occurring adenosine deaminase that includes one or more mutations corresponding to any of the mutations provided herein (e.g., mutations in ecTadA). One of skill in the art will be able to identify the corresponding residue in any homologous protein, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

Provided and described herein are adenosine deaminase variants that have increased efficiency (>50-60%) and specificity. In particular, the adenosine deaminase variants described herein are more likely to edit a desired base within a polynucleotide, and are less likely to edit bases that are not intended to be altered (i.e., “bystanders”).

In some embodiments, the adenosine deaminase is a TadA deaminase. In particular embodiments, the TadA is any one of the TadA described in PCT/US2017/045381 (WO 2018/027078), which is incorporated herein by reference in its entirety.

A wild type TadA(wt) adenosine deaminase has the following sequence (also termed TadA reference sequence):

(SEQ ID NO: 6)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTD

In some embodiments the adenosine deaminase is a full-length E. coli TadA deaminase. For example, in certain embodiments, the adenosine deaminase comprises the amino acid sequence:

(SEQ ID NO: 7)
MRRAFITGVFFLSEVEFSHEYWMRHALTLAKRAWDEREVPVGAVL
VHNNRVIGEGWNRPIGRHDPTAHAEIMALRQGGLVMQNYRLIDAT
LYVTLEPCVMCAGAMIHSRIGRVVFGARDAKTGAAGSLMDVLHHP
GMNHRVEITEGILADECAALLSDFFRMRRQEIKAQKKAQSSTD.

In some embodiments, the adenosine deaminase is from a prokaryote. In some embodiments, the adenosine deaminase is from a bacterium. In some embodiments, the adenosine deaminase is from Escherichia coli (E. coli), Staphylococcus aureus (S. aureus), Salmonella typhimurium (S. typhimurium), Shewanella putrefaciens (S. putrefaciens), Haemophilus influenzae (H. influenzae), Caulobacter crescentus (C. crescentus), Geobacter sulfurreducens (G. sulfurreducens), or Bacillus subtilis. In some embodiments, the adenosine deaminase is from E. coli.

It should be appreciated, however, that additional adenosine deaminases useful in the present application would be apparent to the skilled artisan and are within the scope of this disclosure. For example, the adenosine deaminase may be a homolog of adenosine deaminase acting on tRNA (ADAT). Without limitation, the amino acid sequences of exemplary AD AT homologs include the following:

Staphylococcus aureus (S. aureus) TadA:
(SEQ ID NO: 8)
MGSHMTNDIYFMTLAIEEAKKAAQLGEVPIGAIITKDDEVIARAH
NLRETLQQPTAHAEHIAIERAAKVLGSWRLEGCTLYVTLEPCVMC
AGTIVMSRIPRVVYGADDPKGGCSGSLMNLLQQSNFNHRAIVDKG
VLKEACSTLLTTFFKNLRANKKSTN
Bacillus subtilis (B. subtilis) TadA:
(SEQ ID NO: 9)
MTQDELYMKEAIKEAKKAEEKGEVPIGAVLVINGEIIARAHNLRE
TEQRSIAHAEMLVIDEACKALGTWRLEGATLYVTLEPCPMCAGAV
VLSRVEKVVFGAFDPKGGCSGTLMNLLQEERFNHQAEVVSGVLEE
ECGGMLSAFFRELRKKKKAARKNLSE
Salmonella typhimurium (S. typhimurium)
TadA:
(SEQ ID NO: 10)
MPPAFITGVTSLSDVELDHEYWMRHALTLAKRAWDEREVPVGAVL
VHNHRVIGEGWNRPIGRHDPTAHAEIMALRQGGLVLQNYRLLDTT
LYVTLEPCVMCAGAMVHSRIGRVVFGARDAKTGAAGSLIDVLHHP
GMNHRVEIIEGVLRDECATLLSDFFRMRRQEIKALKKADRAEGAG
PAV
Shewanella putrefaciens (S. putrefaciens)
TadA:
(SEQ ID NO: 11)
MDEYWMQVAMQMAEKAEAAGEVPVGAVLVKDGQQIATGYNLSISQ
HDPTAHAEILCLRSAGKKLENYRLLDATLYITLEPCAMCAGAMVH
SRIARVVYGARDEKTGAAGTVVNLLQHPAFNHQVEVTSGVLAEAC
SAQLSRFFKRRRDEKKALKLAQRAQQGIE
Haemophilus influenzae F3031 (H. influenzae)
TadA:
(SEQ ID NO: 12)
MDAAKVRSEFDEKMMRYALELADKAEALGEIPVGAVLVDDARNII
GEGWNLSIVQSDPTAHAEIIALRNGAKNIQNYRLLNSTLYVTLEP
CTMCAGAILHSRIKRLVFGASDYKTGAIGSRFHFFDDYKMNHTLE
ITSGVLAEECSQKLSTFFQKRREEKKIEKALLKSLSDK
Caulobacter crescentus (C. crescentus) TadA:
(SEQ ID NO: 13)
MRTDESEDQDHRMMRLALDAARAAAEAGETPVGAVILDPSTGEVI
ATAGNGPIAAHDPTAHAEIAAMRAAAAKLGNYRLTDLTLVVTLEP
CAMCAGAISHARIGRVVFGADDPKGGAVVHGPKFFAQPTCHWRPE
VTGGVLADESADLLRGFFRARRKAKI
Geobacter sulfurreducens (G. sulfurreducens)
TadA:
(SEQ ID NO: 14)
MSSLKKTPIRDDAYWMGKAIREAAKAAARDEVPIGAVIVRDGAVI
GRGHNLREGSNDPSAHAEMIAIRQAARRSANWRLTGATLYVTLEP
CLMCMGAIILARLERVVFGCYDPKGGAAGSLYDLSADPRLNHQVR
LSPGVCQEECGTMLSDFFRDLRRRKKAKATPALFIDERKVPPEP
An embodiment of E. Coli TadA (ecTadA)
includes the following:
(SEQ ID NO: 3)
MSEVEFSHEYWMRHALTLAKRARDEREVPVGAVLVLNNRVIGEGW
NRAIGLHDPTAHAEIMALRQGGLVMQNYRLIDATLYVTFEPCVMC
AGAMIHSRIGRVVFGVRNAKTGAAGSLMDVLHYPGMNHRVEITEG
ILADECAALLCYFFRMPRQVFNAQKKAQSSTD

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., ecTadA).

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should be appreciated that any of the mutations provided herein (e.g., based on the ecTadA amino acid sequence of TadA reference sequence) may be introduced into other adenosine deaminases, such as S. aureus TadA (saTadA), or other adenosine deaminases (e.g., bacterial adenosine deaminases). It would be apparent to the skilled artisan how to are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase.

For example, an adenosine deaminase contains a combination of mutations (e.g., V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; or L36H+I76Y+V82G+Y147D+F149Y+Q154S+N157K+D167N), and may contain one or more additional mutations. Additional mutations include, for example, a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in TadA reference sequence, or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, I156D, and/or K157R mutation in TadA reference sequence, or one or more corresponding mutations in another adenosine deaminase.

In some embodiments, the adenosine deaminase comprises one or more of a H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an I156X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:
(A106V_D108N),
(R107C_D108N),
(H8Y_D108N_N127S_D147Y_Q154H),
(H8Y_D108N_N127S_D147Y_E155V),
(D108N_D147Y_E155V),
(H8Y_D108N_N127S),
(H8Y_D108N_N127S_D147Y_Q154H),
(A106V_D108N_D147Y_E155V),
(D108Q_D147Y_E155V),
(D108M_D147Y_E155V),
(D108L_D147Y_E155V),
(D108K_D147Y_E155V),
(D108I_D147Y_E155V),
(D108F_D147Y_E155V),
(A106V_D108N_D147Y),
(A106V_D108M_D147Y_E155V),
(E59A_A106V_D108N_D147Y_E155V),
(E59A cat dead_A106V_D108N_D147Y_E155V),
(L84F_A106V_D108N_H123Y_D147Y_E155V_I156Y),
(L84F_A106V_D108N_H123Y_D147Y_E155V_I156F),
(D103A_D104N),
(G22P_D103A_D104N),
(D103A_D104N_S138A),
(R26G_L84F_A106V_R107H_D108N_H123Y_A142N_A143D_D147Y_E155V_I1 56F),
(E25G_R26G_L84F_A106V_R107H_D108N_H123Y_A142N_A143D_D147Y_E155V_I156F),
(E25D_R26G_L84F_A106V_R107K_D108N_H123Y_A142N_A143G_D147Y_E155V_I156F), (R26Q_L84F_A106V_D108N_H123Y_A142N_D147Y_E155V_I156F),
(E25M_R26G_L84F_A106V_R107P_D108N_H123Y_A142N_A143D_D147Y_E155V_I156F),
(R26C_L84F_A106V_R107H_D108N_H123Y_A142N_D147Y_E155V_I156F),
(L84F_A106V_D108N_H123Y_A142N_A143L_D147Y_E155V_I156F),
(R26G_L84F_A106V_D108N_H123Y_A142N_D147Y_E155V_I156F),
(E25A_R26G_L84F_A106V_R107N_D108N_H123Y_A142N_A143E_D147Y_E155V_I156F),
(R26G_L84F_A106V_R107H_D108N_H123Y_A142N_A143D_D147Y_E155V_I156F),
(A106V_D108N_A142N_D147Y_E155V),
(R26G_A106V_D108N_A142N_D147Y_E155V),
(E25D_R26G_A106V_R107K_D108N_A142N_A143G_D147Y_E155V),
(R26G_A106V_D108N_R107H_A142N_A143D_D147Y_E155V),
(E25D_R26G_A106V_D108N_A142N_D147Y_E155V),
(A106V_R107K_D108N_A142N_D147Y_E155V),
(A106V_D108N_A142N_A143G_D147Y_E155V),
(A106V_D108N_A142N_A143L_D147Y_E155V),
(H36L_R51L_L84F_A106V_D108N_H123Y_S146C_D147Y_E155V_I156F_K157N),
(N37T_P48T_M70L_L84F_A106V_D108N_H123Y_D147Y_I49V_E155V_I156F),
(N37S_L84F_A106V_D108N_H123Y_D147Y_E155V_I156F_K161T),
(H36L_L84F_A106V_D108N_H123Y_D147Y_Q154H_E155V_I156F),
(N72S_L84F_A106V_D108N_H123Y_S146R_D147Y_E155V_I156F),
(H36L_P48L_L84F_A106V_D108N_H123Y_E134G_D147Y_E155V_I156F),
(H36L_L84F_A106V_D108N_H123Y_D147Y_E155V_I156F_K157N)
(H36L_L84F_A106V_D108N_H123Y_S146C_D147Y_E155V_I156F),
(L84F_A106V_D108N_H123Y_S146R_D147Y_E155V_I156F_K161T),
(N37S_R51H_D77G_L84F_A106V_D108N_H123Y_D147Y_E155V_I156F),
(R51L_L84F_A106V_D108N_H123Y_D147Y_E155V_I156F_K157N),
(D24G_Q71R_L84F_H96L_A106V_D108N_H123Y_D147Y_E155V_I156F_K160E
(H36L_G67V_L84F_A106V_D108N_H123Y_S146T_D147Y_E155V_I156F),
(Q71L_L84F_A106V_D108N_H123Y_L137M_A143E_D147Y_E155V_I156F),
(E25G_L84F_A106V_D108N_H123Y_D147Y_E155V_I156F_Q159L),
(L84F_A91T_F104I_A106V_D108N_H123Y_D147Y_E155V_I156F),
(N72D_L84F_A106V_D108N_H123Y_G125A_D147Y_E155V_I156F),
(P48S_L84F_S97C_A106V_D108N_H123Y_D147Y_E155V_I156F),
(W23G_L84F_A106V_D108N_H123Y_D147Y_E155V_I156F),
(D24G_P48L_Q71R_L84F_A106V_D108N_H123Y_D147Y_E155V_I156F_Q159L
(L84F_A106V_D108N_H123Y_A142N_D147Y_E155V_I156F),
(H36L_R51L_L84F_A106V_D108N_H123Y_A142N_S146C_D147Y_E155V_I156 F_K157N),
(N37S_L84F_A106V_D108N_H123Y_A142N_D147Y_E155V_I156F_K161T),
(L84F_A106V_D108N_D147Y_E155V_I156F),
(R51L_L84F_A106V_D108N_H123Y_S146C_D147Y_E155V_I156F_K157N_K16 1T),
(L84F_A106V_D108N_H123Y_S146C_D147Y_E155V_I156F_K161T),
(L84F_A106V_D108N_H123Y_S146C_D147Y_E155V_I156F_K157N_K160E_K1 61T),
(L84F_A106V_D108N_H123Y_S146C_D147Y_E155V_I156F_K157N_K160E),
(R74Q_L84F_A106V_D108N_H123Y_D147Y_E155V_I156F),
(R74A_L84F_A106V_D108N_H123Y_D147Y_E155V_I156F),
(L84F_A106V_D108N_H123Y_D147Y_E155V_I156F),
(R74Q_L84F_A106V_D108N_H123Y_D147Y_E155V_I156F),
(L84F_R98Q_A106V_D108N_H123Y_D147Y_E155V_I156F),
(L84F_A106V_D108N_H123Y_R129Q_D147Y_E155V_I156F),
(P48S_L84F_A106V_D108N_H123Y_A142N_D147Y_E155V_I156F),
(P48S_A142N),
(P48T_I49V_L84F_A106V_D108N_H123Y_A142N_D147Y_E155V_I156F_L157N
(P48T_I49V_A142N),
(H36L_P48S_R51L_L84F_A106V_D108N_H123Y_S146C_D147Y_E155V_I156F_K157N),
(H36L_P48S_R51L_L84F_A106V_D108N_H123Y_S146C_A142N_D147Y_E155V_I156F),
(H36L_P48T_I49V_R51L_L84F_A106V_D108N_H123Y_S146C_D147Y_E155V_156F_K157N),
(H36L_P48T_I49V_R51L_L84F_A106V_D108N_H123Y_A142N_S146C_D147Y_E155V_I156F_K157N),
(H36L_P48A_R51L_L84F_A106V_D108N_H23Y_S146C_D147Y_E155V_I156F_K157N),
(H36L_P48A_R51L_L84F_A106V_D108N_H123Y_A142N_S146C_D147Y_E155V_I156F_K157N),
(H36L_P48A_R51L_L84F_A106V_D108N_H123Y_S146C_A142N_D147Y_E155V_I156F_K157N),
(W23L_H36L_P48A_R51L_L84F_A106V_D108N_H123Y_S146C_D147Y_E155V_I156F_K157N),
(W23R_H36L_P48A_R51L_L84F_A106V_D108N_H123Y_S146C_D147Y_E155V_I156F_K157N),
(W23L_H36L_P48A_R51L_L84F_A106V_D108N_H123Y_S146R_D147Y_E155V_I156F_K161T),
(H36L_P48A_R51L_L84F_A106V_D108N_H123Y_S146C_D147Y_R152H_E155V_I156F_K157N),
(H36L_P48A_R51L_L84F_A106V_D108N_H123Y_S146C_D147Y_R152P_E155V_I156F_K157N),
(W23L_H36L_P48A_R51L_L84F_A106V_D108N_H123Y_S146C_D147Y_R152P_E155V_I156F_K157N),
(W23L_H36L_P48A_R51L_L84F_A106V_D108N_H123Y_A142A_S146C_D147Y_E155V_I156F_K157N),
(W23L_H36L_P48A_R51L_L84F_A106V_D108N_H123Y_A142A_S146C_D147Y_R152P_E155V_I156F_K157N),
(W23L_H36L_P48A_R51L_L84F_A106V_D108N_H123Y_S146R_D147Y_E155V_I156F_K161T),
(W23R_H36L_P48A_R51L_L84F_A106V_D108N_H123Y_S146C_D147Y_R152P_E155V_I156F_K157N),
(H36L_P48A_R51L_L84F_A106V_D108N_H123Y_A142N_S146C_D147Y_R152P_E155V_I156F_K157N).

In some embodiments, the TadA deaminase is TadA variant. In some embodiments, the TadA variant is TadA*7.10. In particular embodiments, the fusion proteins comprise a single TadA*7.10 domain (e.g., provided as a monomer). In other embodiments, the fusion protein comprises TadA*7.10 and TadA(wt), which are capable of forming heterodimers. In one embodiment, a fusion protein as described herein comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 3)
MSEVEFSHEYWMRHALTLAKRARDEREVPVGAVLVLNNRVIGEGW
NRAIGLHDPTAHAEIMALRQGGLVMQNYRLIDATLYVTFEPCVMC
AGAMIHSRIGRVVFGVRNAKTGAAGSLMDVLHYPGMNHRVEITEG
ILADECAALLCYFFRMPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R.

In some embodiments, a variant of TadA*7.10 comprises one or more of alterations selected from the group of L36H, I76Y, V82G, Y147T, Y147D, F149Y, Q154S, N157K, and/or D167N. In some embodiments, a variant of TadA*7.10 comprises V82G, Y147T/D, Q154S, and one or more of L36H, I76Y, F149Y, N157K, and D167N. In other embodiments, a variant of TadA*7.10 comprises a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N.

In some embodiments, an adenosine deaminase variant (e.g., TadA variant) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant (e.g., MSP828) is a monomer comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant (e.g., MSP828) is a monomer comprising V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant (TadA variant) is a monomer comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor of the disclosure comprising an adenosine deaminase variant (e.g., TadA*8) homodimer comprising two adenosine deaminase domains (e.g., TadA*8) each having one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, an adenosine deaminase variant is a homodimer comprising two adenosine deaminase domains (e.g., TadA*7.10) each having one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a homodimer comprising two adenosine deaminase variant domains (e.g., MSP828) each having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a homodimer comprising two adenosine deaminase domains (e.g., TadA*7.10) each having a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*7.10) comprising one or more of the following alterations L36H, 176Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a wild-type adenosine deaminase domain and an adenosine deaminase variant domain (e.g., MSP828) having the following alterations V82G, Y147T/D, Q154S, and one or more of L36H, 176Y, F149Y, N157K, and D167N, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*7.10) comprising a combination of alterations selected from the group of: V82G+Y147T+Q154S; 176Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+176Y+V82G+Y147T+Q154S+N157K; 176Y+V82G+Y147D+F149Y+Q154S+D167N; L36H+176Y+V82G+Y147D+F149Y+Q154S+N157K+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; 176Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*7.10) comprising one or more of the following alterations L36H, I76Y, V82G, Y147T, Y147D, F149Y, Q154S, N157K, and/or D167N, relative to TadA*7.10, the TadA reference sequence, or a corresponding mutation in another TadA. In some embodiments, an adenosine deaminase variant is a heterodimer comprising a TadA*7.10 domain and an adenosine deaminase variant domain (e.g., MSP828) having the following alterations V82G, Y147T/D, Q154S, and one or more of L36H, I76Y, F149Y, N157K, and D167N, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*7.10) comprising a combination of alterations selected from the group of: V82G+Y147T+Q154S; I76Y+V82G+Y147T+Q154S; L36H+V82G+Y147T+Q154S+N157K; V82G+Y147D+F149Y+Q154S+D167N; L36H+V82G+Y147D+F149Y+Q154S+N157K+D167N; L36H+I76Y+V82G+Y147T+Q154S+N157K; I76Y+V82G+Y147D+F149Y+Q154S+D167N; L36H+I76Y+V82G+Y147D+F149Y+Q154S+N157K+D167N, relative to TadA*7.10, the TadA reference sequence, or a corresponding mutation in another TadA.

In some embodiments, the TadA*8 is a variant as shown in Tables 8A, 10, 11, or 13. Tables 8A, 10, 11, and 13 show certain amino acid position numbers in the TadA amino acid sequence and the amino acids present in those positions in the TadA-7.10 adenosine deaminase. Tables 8A, 10, 11, and 13 also show amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

In particular embodiments, an adenosine deaminase heterodimer can comprise a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity:

(SEQ ID NO: 16)
MSEVEFSHEYWMRHALTLAKRARDEREVPVGAVLVLNNRVIGEGW
NRAIGLHDPTAHAEIMALRQGGLVMQNYRLIDATLYVTFEPCVMC
AGAMIHSRIGRVVFGVRNAKTGAAGSLMDVLHYPGMNHRVEITEG
ILADECAALLCTFFRMPRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein as described and/or exemplified herein comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24

TABLE 5
Additional TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 6. Table 6 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 6
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In one embodiment, a fusion protein as described herein comprises a wild-type TadA is linked to an adenosine deaminase variant described herein, which is linked to Cas9 nickase. In particular embodiments, the fusion proteins comprise a single variant TadA domain (e.g., provided as a monomer). In other embodiments, the fusion protein comprises a variant TadA and TadA(wt), which are capable of forming heterodimers.

In some embodiments, the TadA variant is truncated. In some embodiments, the truncated TadA is missing 1, 2, 3, 4, 5, 6, 7, 8, 9, 10, 11, 12, 13, 14, 15, 6, 17, 18, 19, or 20 N-terminal amino acid residues relative to the full length TadA variant. In some embodiments, the truncated TadA variant is missing 1, 2, 3, 4, 5, 6, 7, 8, 9, 10, 11, 12, 13, 14, 15, 6, 17, 18, 19, or 20 C-terminal amino acid residues relative to the full length TadA variant. In some embodiments the adenosine deaminase variant is a full-length TadA variant.

In particular embodiments, a TadA*8 comprises one or more mutations at any of the following positions shown in bold. In other embodiments, a TadA*8 comprises one or more mutations at any of the positions shown with underlining:

(SEQ ID NO: 3)
MSEVEFSHEY WMRHALTLAK RARDEREVPV GAVLVLNNRV IGEGWNRAIG 50
LHDPTAHAEI MALRQGGLVM QNYRLIDATL YVTFEPCVMC AGAMIHSRIG 100
RVVFGVRNAK TGAAGSLMDV LHYPGMNHRV EITEGILADE CAALLCYFFR 150
MPRQVFNAQK KAQSSTD

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA.

In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA. In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K. The one or more alternations are shown in the sequence above in underlining and bold font.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R.

In some embodiments, an adenosine deaminase comprises one or more of the following combinations of alterations: Q71M+V82S+Y123H+Y147R+Q154R; E25F+I76Y+V82S+Y123H+Y147R+Q154R; 176Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/2020/049975, which is incorporated herein by reference for its entirety. In one embodiment, a fusion protein as described herein comprises a wild-type TadA is linked to an adenosine deaminase variant described herein (e.g., TadA variant), which is linked to Cas9 nickase. In particular embodiments, the fusion proteins comprise a single TadA variant domain (e.g., provided as a monomer). In other embodiments, the base editor comprises TadA*8 and TadA(wt), which are capable of forming heterodimers.

In particular embodiments, the fusion proteins comprise a single (e.g., provided as a monomer) TadA variant domain. In some embodiments, the TadA variant is linked to a Cas9 nickase. In some embodiments, the fusion proteins described herein comprise as a heterodimer of a wild-type TadA (TadA(wt)) linked to a TadA variant. In other embodiments, the fusion proteins described herein comprise as a heterodimer of a TadA*7.10 linked to a TadA variant. In some embodiments, the fusion protein comprises a TadA variant monomer. In some embodiments, the fusion protein comprises a heterodimer of a TadA variant and a TadA(wt). In some embodiments, the fusion protein comprises a heterodimer of a TadA variant and TadA*7.10. In some embodiments, the fusion protein comprises a heterodimer of two TadA variants. In some embodiments, the TadA variant is selected from Table 5, 6, infra or any other TadA variant provided herein.

In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### Use of Nucleobase Editors to Target Nucleotides in the G6PC Gene

The suitability of nucleobase editors that target a nucleotide in the G6PC gene is evaluated as described herein.

The activity of the nucleobase editor is assessed as described herein, i.e., by sequencing the target gene to detect alterations in the target sequence. For Sanger sequencing, purified PCR amplicons are cloned into a plasmid backbone, transformed, miniprepped and sequenced with a single primer. Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq).

In some embodiments, the nucleobase editors are used to target polynucleotides of interest. In one embodiment, a nucleobase editor as described herein is delivered to cells (e.g., hepatocytes) in conjunction with a guide RNA that is used to target a nucleic acid sequence, e.g., a G6PC polynucleotide harboring GSD1a-associated mutations, thereby altering the target gene, i.e., G6PC.

In some embodiments, a base editor is targeted by a guide RNA to introduce one or more edits to the sequence of a gene of interest (e.g. G6PC). In some embodiments, the one or more alterations are introduced into the glucose-6-phosphatase (G6PC) gene. In some embodiments the one or more alterations is R83C. In some embodiments, the one or more alterations is Q347X. In some embodiments, the alteration is introduced into a representative Homo sapiens G6PC protein, found under NCBI Reference Sequence No. AAA16222.1. In some embodiments, the alteration is introduced into a representative Homo sapiens G6PC nucleic acid sequence, found under GenBank Reference Sequence No. U01120.1.

### Therapeutic Applications

The NLS-gRNA described herein can be used in a gene editing system for various therapeutic applications. Accordingly, in some embodiments, a method of treating a disorder or a disease in a subject in need thereof is provided, the method comprising administering to the subject a NLS-gRNA described herein with a gene editing system. Various gene editing systems are known in the art and include for example CRISPR-Cas9, Cpf1, SaCas9, Cas12. The NLS-gRNA described herein can be used with any gene editing system. For example, Cas protein is from an organism from a genus comprising Streptococcus, Campylobacter, Nitratifr actor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospira, Lachnospiraceae, Clostridiaridium, Leptotrichia, Francisella, Legionella, Alicyclobacillus, Methanomethyophilus, Porphyromonas, Prevotella, Bacteroidetes, Helcococcus, Leptospira, Desulfovibrio, Desulfonatronum, Opitutaceae, Tuberibacillus, Bacillus, Brevibacilus, Methylobacterium, Butyvibrio, Perigrinibacterium, Pareubacterium, Moraxella, Thiomicrospira or Acidaminococcus. In particular embodiments, the Cpf1 effector protein is selected from an organism from a genus selected from Eubacterium, Lachnospiraceae, Leptotrichia, Francisella, Methanomethyophilus, Porphyromonas, Prevotella, Leptospira, Butyvibrio, Perigrinibacterium, Pareubacterium, Moraxella, Thiomicrospira or Acidaminococcus.

Non-limiting examples of Cas species include Streptococcus pyogenes, Streptococcus thermophiles, Streptococcus aureas Neisseria meningitides, Treponema denticola, Francisella tularensis, Campylobacter jejuni, Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Geobacillus stearothermophilus, Streptococcus constellatus, Sharpea spp. isolate RUG017, Veillonella parvula, Ezakiella peruensis, Lactobacillus fermentum strain AF15-40LB and Peptoniphilus sp. Marseille-P3761.

In some embodiments, the NLS-gRNA described herein can be used in conjunction with a gene editing system to treat various diseases and disorders, e.g., genetic disorders (e.g., monogenetic diseases), diseases that can be treated by nuclease activity, and various cancers, etc.

In some embodiments, the NLS-gRNA described herein can be used in conjunction with a gene editing system to edit a target nucleic acid to modify the target nucleic acid (e.g., by inserting, deleting, or mutating one or more nucleic acid residues). For example, in some embodiments a CRISPR systems is used with the NLS-gRNA described herein and comprises an exogenous donor template nucleic acid (e.g., a DNA molecule or a RNA molecule), which comprises a desirable nucleic acid sequence. Upon resolution of a cleavage event induced with the CRISPR system, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the NLS-gRNA described herein is used in conjunction with a gene editing system to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA).

In one aspect, NLS-gRNA described herein can be used in conjunction with a gene editing system for treating a disease caused by overexpression of RNAs, toxic RNAs, and/or mutated RNAs (e.g., splicing defects or truncations).

In some embodiments, the NLS-gRNA described herein can be used in conjunction with a gene editing system to target trans-acting mutations affecting RNA-dependent functions that cause various diseases.

In some embodiments, the NLS-gRNA described herein can be used in conjunction with a gene editing system to target mutations disrupting the cis-acting splicing codes that can cause splicing defects and diseases.

The NLS-gRNA described herein can be used in conjunction with a gene editing system can for antiviral activity, in particular against RNA viruses. For example, to target viral RNAs using suitable NLS-gRNA selected to target viral RNA sequences.

The NLS-gRNA described herein can be used in conjunction with a gene editing system to treat a cancer in a subject (e.g., a human subject). For example, by targeting a RNA molecule that is aberrant (e.g., comprises a point mutation or are alternatively-spliced) and found in cancer cells to induce cell death in the cancer cells (e.g., via apoptosis).

The NLS-gRNA described herein can be used in conjunction with a gene editing system to treat an infectious disease in a subject. For example, through targeting a RNA molecule expressed by an infectious agent (e.g., a bacteria, a virus, a parasite or a protozoan) in order to target and induce cell death in the infectious agent cell. The synthetic guide RNA described herein can be used in conjunction with a gene editing system to treat diseases where an intracellular infectious agent infects the cells of a host subject.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, the DNA-targeting RNA and/or site-directed modifying polypeptide and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, a DNA-targeting RNA and/or site-directed modifying polypeptide and/or donor polynucleotide are administered directly to the individual. A DNA-targeting RNA and/or site-directed modifying polypeptide and/or donor polynucleotide may be administered by any of a number of well-known methods in the art for the administration of peptides, small molecules and nucleic acids to a subject. A DNA-targeting RNA and/or site-directed modifying polypeptide and/or donor polynucleotide can be incorporated into a variety of formulations. More particularly, a DNA-targeting RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a DNA-targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a DNA-targeting RNA and/or site-directed modifying polypeptide and/or donor polynucleotide are provided. As discussed above with regard to ex vivo methods, an effective amount or effective dose of a DNA-targeting RNA and/or site-directed modifying polypeptide and/or donor polynucleotide in vivo is the amount to induce a 2 fold increase or more in the amount of recombination observed between two homologous sequences relative to a negative control, e.g. a cell contacted with an empty vector or irrelevant polypeptide. The amount of recombination may be measured by any convenient method, e.g. as described above and known in the art. The calculation of the effective amount or effective dose of a DNA-targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated. In some embodiments, an exemplary dose of between about 0.01 to 1 mpk is used.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a DNA-targeting RNA and/or site-directed modifying polypeptide and/or donor polynucleotide may be obtained from a suitable commercial source. As a general proposition, the total pharmaceutically effective amount of the a DNA-targeting RNA and/or site-directed modifying polypeptide and/or donor polynucleotide administered parenterally per dose will be in a range that can be measured by a dose response curve.

Therapies based on a DNA-targeting RNA and/or site-directed modifying polypeptide and/or donor polynucleotides, i.e. preparations of a DNA-targeting RNA and/or site-directed modifying polypeptide and/or donor polynucleotide to be used for therapeutic administration, must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The therapies based on a DNA-targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The NLS-gRNA described herein, along with a desired gene editing system components, can be delivered to a cell of interest by various delivery systems such as vectors, carriers, e.g., lipid nanoparticles.

The NLS-gRNA described herein can be delivered by nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 2 (below).

TABLE 2
Lipids Used for Gene Transfer

| Lipid | Abbreviation | Feature |
|---|---|---|
| 1,2-Dioleoyl-sn-glycero-3- | DOPC | Helper |
| phosphatidylcholine | | |
| 1,2-Dioleoyl-sn-glycero-3- | DOPE | Helper |
| phosphatidylethanolamine | | |
| Cholesterol | | Helper |
| N-[1-(2,3-Dioleyloxy)prophyl]N,N,N- | DOTMA | Cationic |
| trimethylammonium chloride | | |
| 1,2-Dioleoyloxy-3-trimethylammonium- | DOTAP | Cationic |
| propane | | |
| Dioctadecylamidoglycylspermine | DOGS | Cationic |
| N-(3-Aminopropyl)-N,N-dimethyl-2,3- | GAP-DLRIE | Cationic |
| bis(dodecyloxy)-1-propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido- | DOSPA | Cationic |
| ethyl]-N,N-dimethyl-1-propanaminium | | |
| trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium | | |
| chloride | | |
| rac-[2(2,3-Dihexadecyloxypropyl- | CLIP-6 | Cationic |
| oxymethyloxy)ethyl]trimethylammoniun | | |
| bromide | | |
| Ethyldimyristoylphosphatidylcholine | EDMPC | Cationic |
| 1,2-Distearyloxy-N,N-dimethyl-3- | DSDMA | Cationic |
| aminopropane | | |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho | DSEPC | Cationic |
| sphocholine | | |
| N-Palmitoyl D-erythro-sphingosyl carbamoyl- | CCS | Cationic |
| spermine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane- | CDAN | Cationic |
| 1,9-diamine | | |
| 2-(3-[Bis(3-amino-propyl)- | RPR209120 | Cationic |
| amino]propylamino)-N-ditetradecylcarbamoylme- | | |
| ethyl-acetamide | | |
| 1,2-dilinoleyloxy-3-dimethylaminopropane | DLinDMA | Cationic |
| 2,2-dilinoleyl-4-dimethylaminoethyl- | DLin-KC2- | Cationic |
| [1,3]-dioxolane | DMA | |
| dilinoleyl-methyl-4-dimethylaminobutyrate | DLin-MC3- | Cationic |
| | DMA | |

Table 3 lists exemplary polymers for use in gene transfer and/or nanoparticle formulations.

TABLE 3
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 4 summarizes delivery methods for a polynucleotide encoding a Cas9 described herein.

TABLE 4

| | | Delivery into | Duration of | Genome | Type of |
|---|---|---|---|---|---|
| Delivery | Vector/Mode | Non-Dividing Cells | Expression | Integration | Molecule Delivered |
| Physical | (e.g., electroporation, | YES | Transient | NO | Nucleic Acids |
| | particle gun, Calcium | | | | and Proteins |
| | Phosphate transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very Transient | NO | DNA |
| | Herpes Simplex | YES | Stable | NO | DNA |
| | Virus | | | | |
| Non-Viral | Cationic | YES | Transient | Depends on what | Nucleic Acids |
| | Liposomes | | | is delivered | and Proteins |
| | Polymeric | YES | Transient | Depends on what | Nucleic Acids |
| | Nanoparticles | | | is delivered | and Proteins |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian Virus- | YES | Transient | NO | Nucleic Acids |
| | like Particles | | | | |
| | Biological | YES | Transient | NO | Nucleic Acids |
| | liposomes: | | | | |
| | Erythrocyte Ghosts | | | | |
| | and Exosomes | | | | |

In another aspect, the delivery of genome editing system including the NLS-gRNA describe herein may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive the CRISPR system (e.g., including the synthetic gRNA described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, separate promoters drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas9 can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 pg of lentiviral transfer plasmid (pCasES10) and the following packaging plasmids: 5 pg of pMD2.G (VSV-g pseudotype), and 7.5 pg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a NLS-gRNA or a Cas9-encoding mRNA, can be delivered in the form of RNA. Cas9 encoding mRNA can be generated using in vitro transcription. For example, Cas9 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas9 sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising gene editing system (e.g., including the NLS-gRNA described herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar, (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas9 described herein) are provided as part of a pharmaceutical composition. In some embodiments, the pharmaceutical composition comprises any of the fusion proteins provided herein (e.g., including the nucleobase editor described herein comprising LubCas9). In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Kits

In one aspect, the NLS-gRNA described herein can be provided and or produced by a kit containing any one or more of the elements disclosed in the above methods and compositions. For example, a kit may include a NLS-gRNA, a ligase, and suitable buffering reagents.

In some embodiments, the kit further comprises a nucleobase editor.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1: Ex Vivo Efficacy of NLS-gRNA

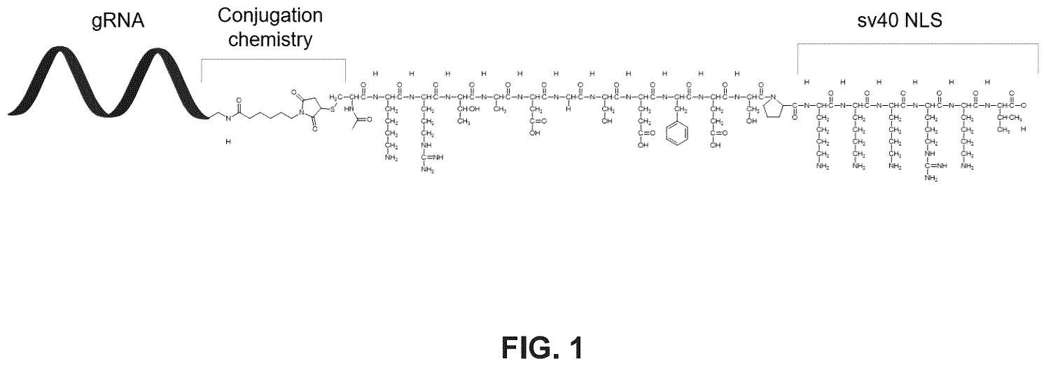

This example describes an exemplary gRNA conjugated to NLS (NLS-gRNA) of the present invention and its efficacy ex vivo. A peptide comprising the NLS sequence and a peptide spacer was synthesized by solid-phase peptide synthesis. The synthesized peptide was conjugated to the 3′ end of the gRNA via thiol group, as shown in FIG. 1. As one of ordinary skill in the art would appreciate, the linker and the peptide spacer can be modified in the practice of the present invention. Additionally, the sequence of the NLS, gRNA, and/or linker can be modified.

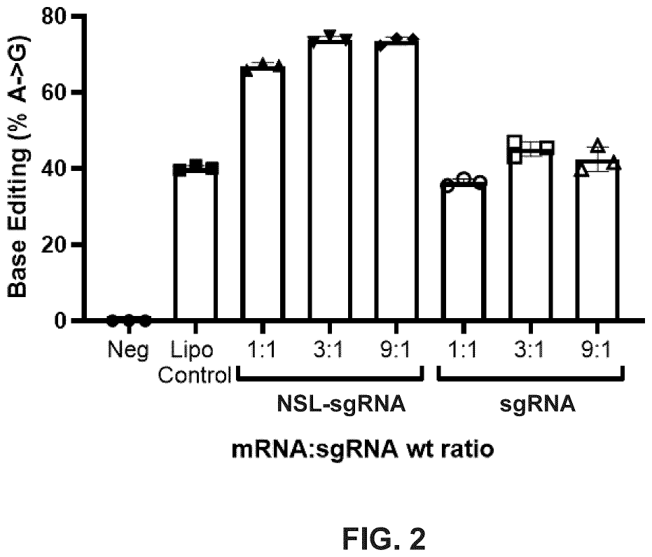

NLS-sgRNA was prepared and formulated in lipid nanoparticles with mRNA encoding a CRISPR-Cas9 based editor. The formulation was delivered to hepatocytes at three different ratios of mRNA:sgRNA (1:1, 3:1, and 9:1). As shown in FIG. 2, NLS-sgRNA showed a significantly higher base editing efficiency as compared to gRNA without the NLS sequence.

The data in this example shows that CRISPR-Cas system (e.g., base editing) can be improved by using a gRNA that is conjugated to a NLS sequence. Without wishing to be bound by a particular theory, the improvement in CRISPR-Cas system may be due in part to better trafficking of the NLS-gRNA to the nucleus which protects gRNA from cytosolic RNases, increased local concentration of gRNA and therefore ribonucleic acid complex (RNP) formation, and higher rate of import to the nucleus. Furthermore, the cationic NLS sequence may act in part by promoting endosomal escape.

### Example 2: In Vivo Efficacy of NLS-gRNA

This example illustrates that NLS-gRNA significantly improves base editing in vivo, even as compared to highly modified gRNA. In this example, spCas9 gRNAs were used with an adenine base editor (ABE) comprising an spCas9 nickase and adenosine deaminase.

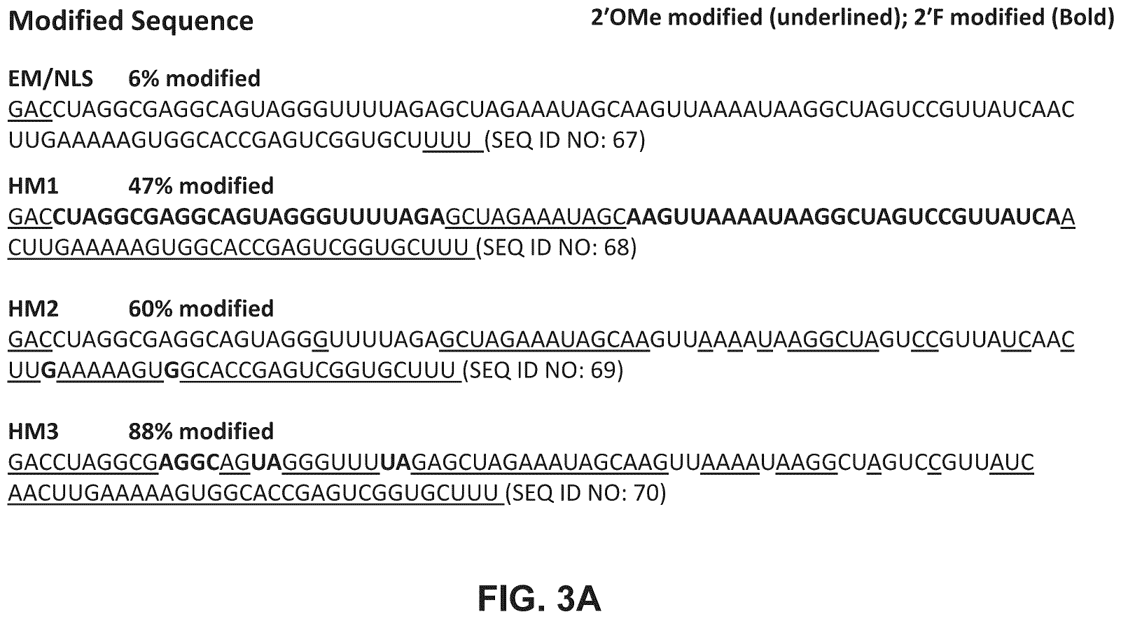

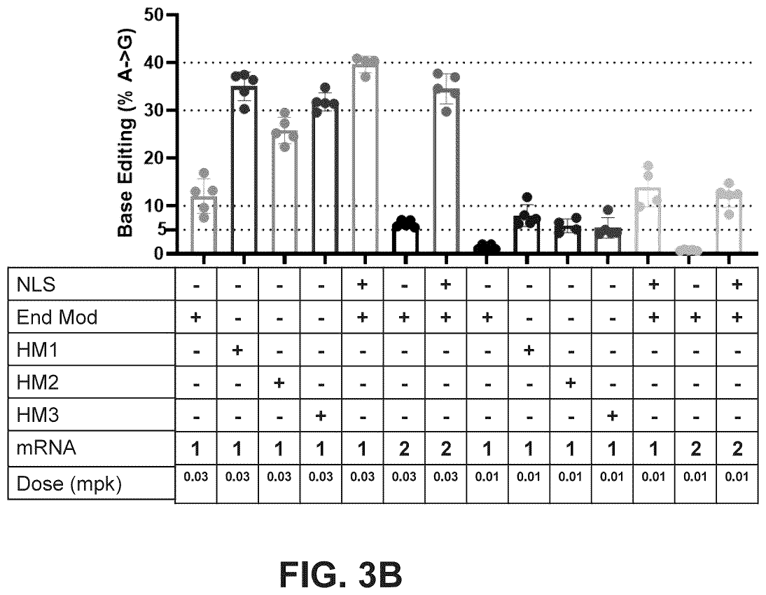

gRNAs with various modifications were prepared. As shown in FIG. 3A, an end-modified (EM) gRNA comprises 6% modifications, a heavy mod 1 (HM1) gRNA comprises 47% modification, a heavy mod 2 (HM2) gRNA comprises 60% modification, and a heavy mod 3 (HM3) gRNA comprises 88% modification. NLS-gRNA comprises NLS sequence conjugated to the 3′ end of the gRNA and 6% modification. Two different mRNAs, both encoding the same bae editor were prepared. As compared to the mRNA 2, mRNA 1 is codon-optimized, with 3′ and 5′ UTR sequences. Various combinations of the gRNAs with either mRNA1 or mRNA2 were formulated in LNPs and were delivered to mice at sub-saturating dose of 0.03 mpk or 0.01 mpk, as shown in FIG. 3B.

The results show that NLS-gRNA exhibited higher base editing efficacy as compared to all EM, HM1, HM2, or HM3 gRNAs. Particularly, even at ultra-low doses (0.01 mpk), base editing was visible for NLS-gRNA, and was significantly higher than heavily modified (HM1, HM2, and HM3) gRNAs. Additionally, combining NLS-gRNA with less potent mRNA (mRNA2) compensated for the quality of mRNA—while the base editing efficiency of mRNA2 with end-modified gRNA was about 5%, substituting the gRNA with NLS-gRNA increased the base editing efficiency to greater than 30%.

### Example 3: Efficacy of NLS-gRNA in Non-Human Primates (NHP)

This example illustrates that the improvement in base editing efficiency by using NLS-gRNA is also observed in NHPs. In this example, spCas9 gRNAs were used with an spCas9-based adenine base editor (ABE).

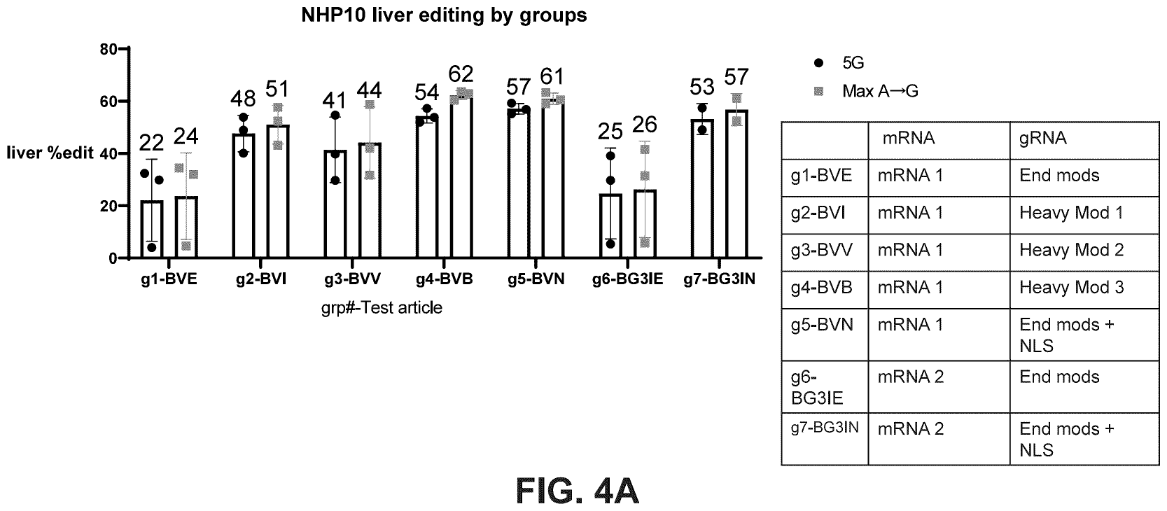

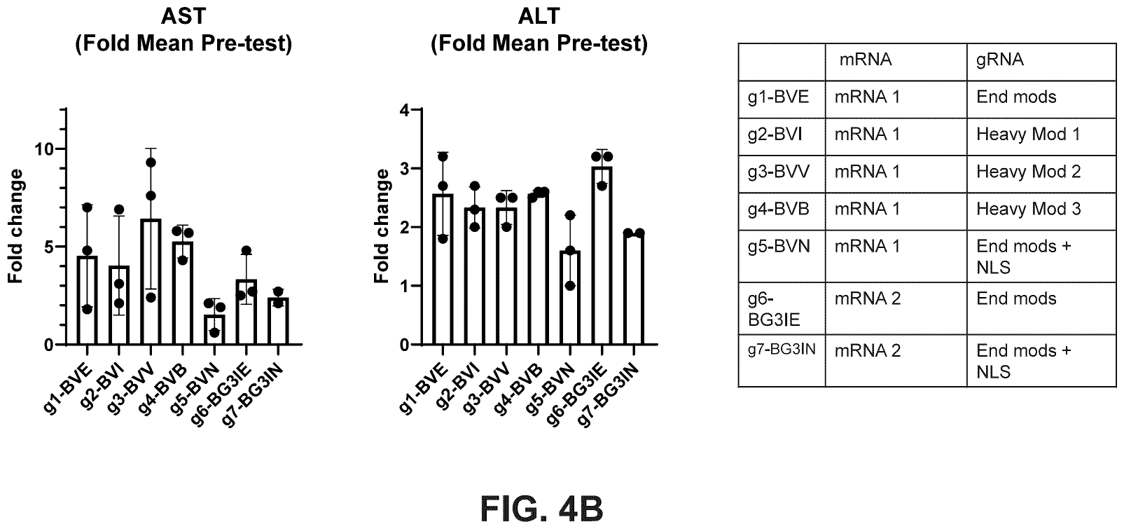

Various gRNAs and mRNA encoding a base editor were formulated in lipid nanoparticles as shown in FIG. 4A. The formulations were delivered to NHPs at 1.0 mpk, and base editing efficiency was determined in liver. The results show that NLS-gRNA with mRNA1 (g5-BVN) and HM3 gRNA with mRNA1 (g4-BVB) exhibited the highest base editing efficiency, followed by g2-BVI, g3-BVV, and g1-BVE. Notably, NLS-gRNA with end modifications (g1-BVN and g7-BG3IN) showed more than two-fold base editing efficiency as compared to respective end-modified gRNA without NLS (compare to g1-BVE and g6-BG3IE, respectively).

Next, toxicology study was performed in the NHPs. To evaluate clinical pathology, alanine aminotransferase (ALT) and aspartate aminotransferase (AST) levels were measured. Higher levels of ALT and AST correlate with liver damage. As shown in FIG. 4B, minimal to mild increases in AST and/or ALT were observed 24 hr post-dose for all test articles. Notably, g5-BVN, which comprises NLS-gRNA with end modification showed the lowest AST and ALT increases. Additionally, no other significant changes in clinical pathology parameters were observed.

Overall, data in this example illustrates that NLS-gRNA improves CRISPR-Cas system (e.g., base editing efficiency) in NHPs, with decreased toxicity.

### Example 4: Application of NLS-gRNA in saCas9

This example illustrates that NLS-gRNA can be applied to various Cas proteins. In this particular example, a Staphylococcus aureus Cas9 (saCas9) was used. Notably, saCas9 requires a unique guide that is not compatible with spCas9 editing shown in previous examples.

Glycogen storage disease type 1a (GSD1a) is caused by a mutation in the glucose-6-phosphatase (G6PC) gene, which affects about 80% of patients with GSD1a. The R83C mutation affects about 900 US patients annually diagnosed with Glycogen storage disease type 1a (GSD1a). This mutation is a single base substitution that introduces a cysteine at position 83 (R83C) of the G6PC protein. A precise correction of R83C will likely restore expression of G6PC and normalize glucose metabolism.

gRNA were prepared and its purity was determined. gRNAs with two different backbone chemistry were used in the study (sg029 vs. sg093). Sg093 guides have end modifications with 2′-OMe and phosphothioate modifications). Various gRNAs and mRNA encoding a base editor were formulated in LNPs at 1:1 ratio of gRNA:mRNA. Adult transgenic mice heterozygous for huG6PC-R83C were administered LNP formulations at a sub-saturating dose of 1 mpk.

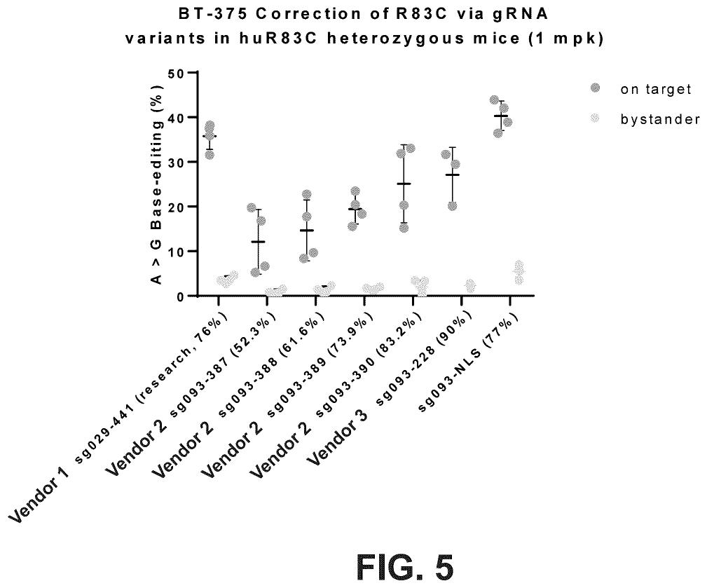

FIG. 5 shows a correlation between base editing efficiency and purity of gRNA, with 80% purity yielding maximum base editing levels. Additionally, NLS-gRNA showed an improvement in potency with spCas9 protein relative to other sg093 guides without NLS sequence, illustrating that NLS-gRNA of the present invention can be applied across multiple Cas proteins.

### Example 5: In Vivo Base Editing Correction of Metabolic Defects in GSD1a R83C Mice Using NLS-gRNA

In this example, variants of Adenine base editors (ABEs) were used in connection with NLS-gRNA to correct metabolic defects in GSD1a R83C mice. The R83C mutation introduces a single G>A conversion in the g6pc gene. ABEs in combination with NLS-gRNA as described herein effect the programmable conversion of A to G in genomic DNA, thus supporting their utility to correct this mutation.

The G6PC gRNA sequence hybridizes to the complement of the G6PC target sequence shown below:

(SEQ ID NO: 1)
CAGTATGGACACTGTCCAAA GAGAAT

The NNGRRT PAM sequence (i.e., Staphylococcus aureus Cas9 (saCas9)) is underlined above. The gRNA sequence is as follows: CAGUAUGGACACUGUCCAAA (SEQ ID NO: 2).

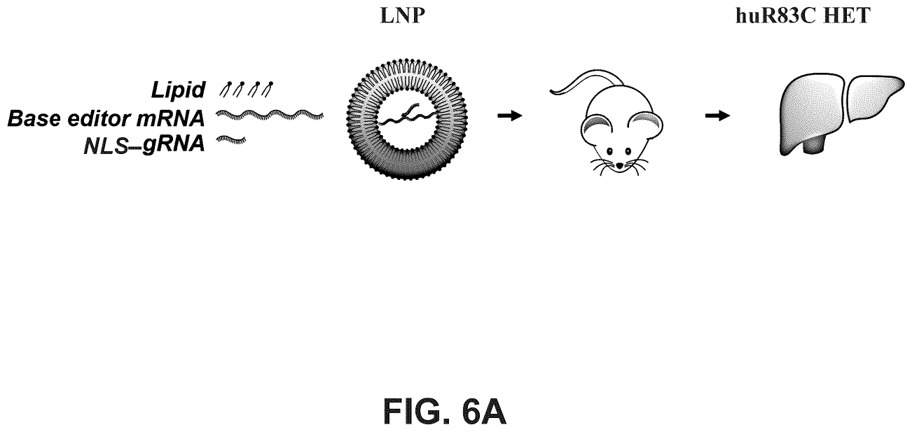

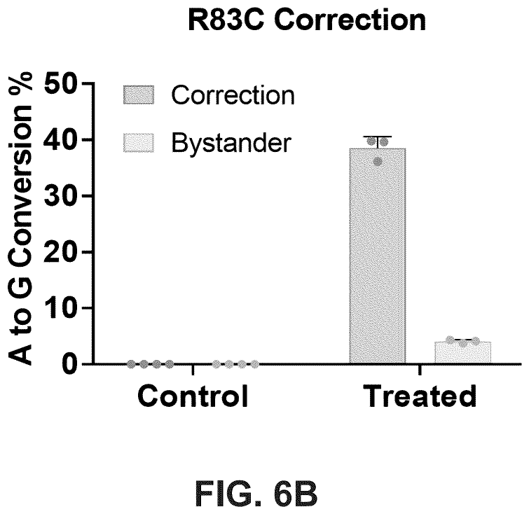

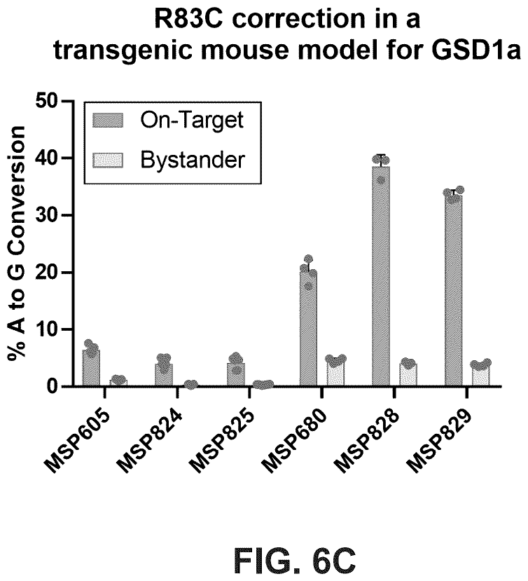

The base-editing efficiency of adenosine deaminase base editors (ABE) using TadA variants MSP605, MSP824, MSP825, MSP680, MSP828, and MSP829 (see Table 1) and saCas9n was evaluated in vivo using a transgenic mouse model heterozygous for huG6PC, harboring the R83C mutation for Glycogen storage disease type 1a (GSD1a) (FIGS. 6B and 6C). The use of saCas9 for efficient in vivo genome editing and exemplification of an saCas9 sgRNA scaffold are described in A. Ran et al. (2015, Nature, Vol. 520, pages 186-191).

TABLE 1
Adenosine Deaminase Base Editor Variants

| TadA | |
|---|---|
| Variant | mRNA base-editor variant |
| MSP605 | dimeric TadA-ABE7.10 (Y147T + Q154S + |
| | V82G)-saCas9n |
| MSP824 | dimeric TadA-ABE7.10 (Y147D + Q154S + |
| | V82G + F149Y + D167N)-saCas9n |
| MSP825 | dimeric TadA-ABE7.10 (Y147D + Q154S + |
| | V82G + L36H + N157K + F149Y + D167N)-saCas9n |
| MSP680 | monomeric TadA-ABE7.10 (Y147T + Q154S + |
| | V82G + I76Y)-saCas9n |
| MSP828 | monomeric TadA-ABE7.10 (Y147D + Q154S + |
| | V82G + I76Y + F149Y + D167N)-saCas9n |
| MSP829 | monomeric TadA-ABE7.10 (Y147D + Q154S + |
| | V82G + I76Y + L36H + N157K + F149Y + D167N)-saCas9n |

FIG. 6A depicts the in vivo workflow used to introduce the base editors into the transgenic mice. Lipid nanoparticles (LNP) carrying base editor mRNA and NLS-gRNA were dosed via intravenous (IV) injection into the transgenic mice at a dose of 1 mg/kg. Next-generation sequencing data from whole-liver extracts revealed significant correction for R83C (FIGS. 6B and 6C). TadA variant MSP828 demonstrated about 40% precise correction of the R83C mutation, with low bystander editing. This level of mutation correction is expected to restore glucose homeostasis.

### Example 6: In Vivo Base Editing Correction of Metabolic Defects in GSD1a R83C Mice GSD1a Overview

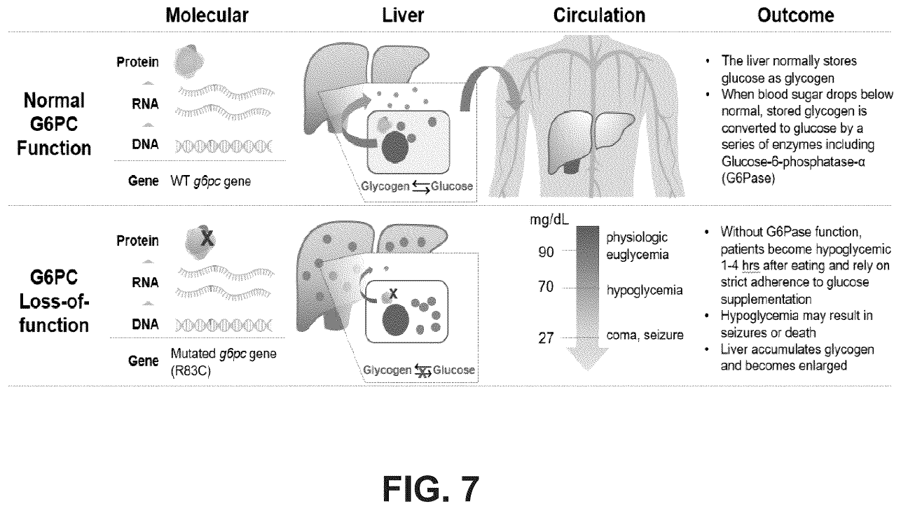

As depicted schematically in FIG. 7, (GSD1a) is an autosomal recessive disorder caused by mutations in the G6PC gene. The most prevalent pathogenic mutation identified in Caucasian GSD1a patients is R83C, located in the active site of the enzyme and associated with inactivation of G6Pase. A loss of G6Pase function can result in life-threatening hypoglycemia, seizures and even death. To mitigate hypoglycemia, patients must maintain strict and frequent adherence to glucose supplementation through day and night, by way of a slow glucose release formula. One missed or delayed dose can result in emergency hypoglycemia. Among many complications, enlarged liver, accumulation of uric acid, lactate, and lipids are common in GSD1a patients.

### Utility of the Described Base Editors for Generating Permanent and Predictable Single Nucleotide Substitutions

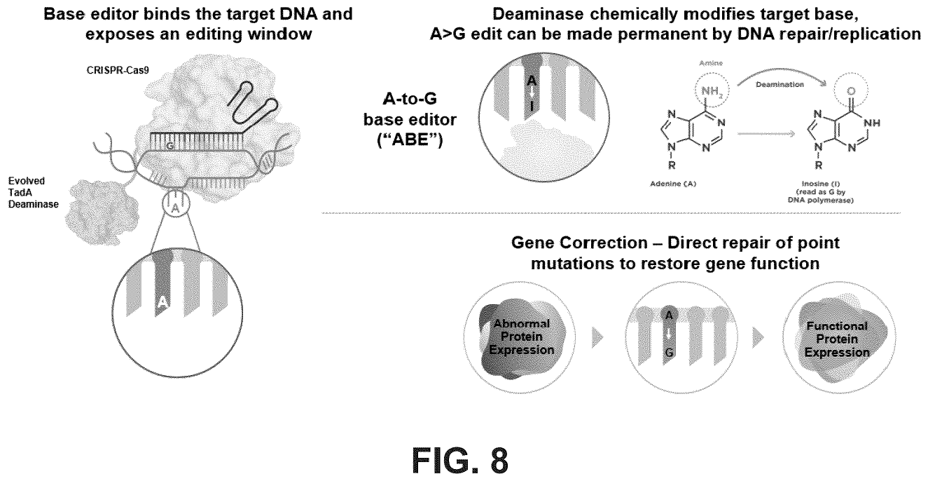

The R83C mutation introduces a single G>A conversion in the g6pc gene. Adenine base editors (ABEs) as described herein effect the programmable conversion of A to G in genomic DNA, thus supporting their utility to correct this mutation. As shown schematically in FIG. 8, the adenine base editor is a fusion protein containing an evolved TadA deaminase connected to CRISPR-Cas enzyme. The base editor binds to target DNA that is complementary to the guide-RNA (superimposed on the CRISPR-Cas9 enzyme) and exposes a stretch of single-stranded DNA. The deaminase converts the target adenine into inosine, and the Cas enzyme nicks the opposite strand, which is then repaired, completing the base pair conversion. Thus, the direct repair of a point mutation has the potential for restoration of gene function.

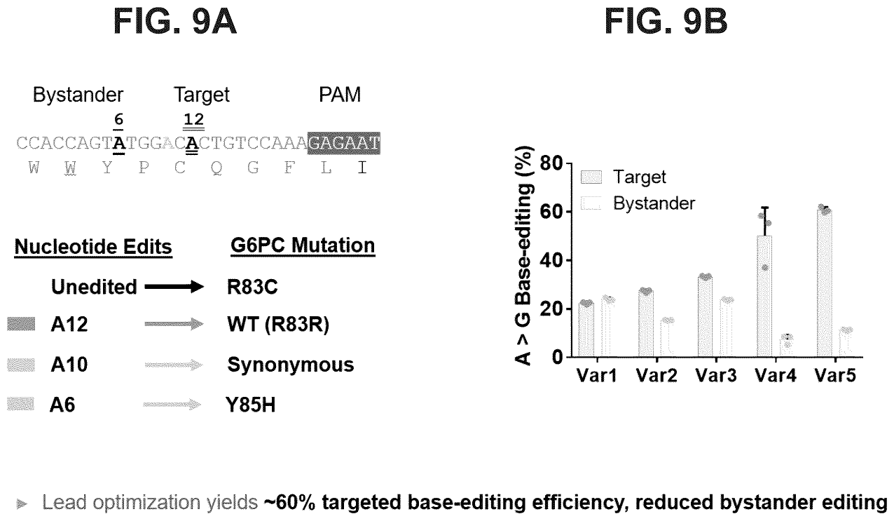

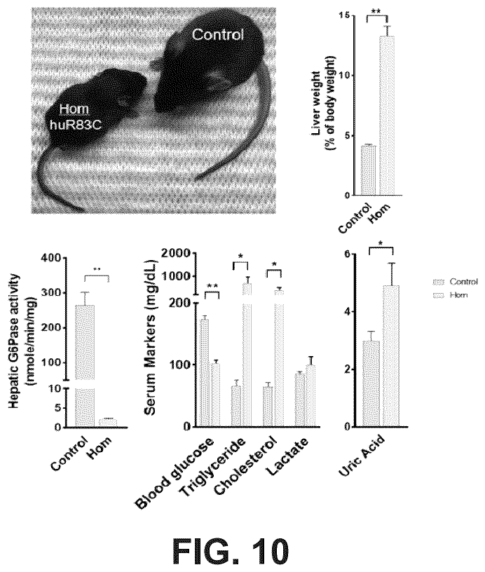

In this Example, base-editors for A>G conversion in the g6pc gene were optimized for correction of R83C. Shown in FIG. 9A is the target DNA sequence (CCACCAGTATGGACACTGTCCAAAGAGAAT (SEQ ID NO: 17)) and underlying amino acid translation for the GSD1a R83C mutation (WWYPCQGFLI; SEQ ID NO: 18). The target nucleobase to be edited is represented by double underlining, at position 12. The editing window also includes a possible bystander, shown represented by single underlining at position 6. An edit that may result in a synonymous conversion is shown at position 10.

For screening, a HEK293 cell line that expressed the G6PC transgene harboring the R83C mutation was generated and was transfected with base-editor mRNA and gRNA. Allele frequencies were assessed by high-throughput targeted amplicon Next-Generation Sequencing. Variants 1-5 represent a combination of gRNA and base-editor RNA, engineered for optimized target correction. Variant 5 yielded approximately 60% targeted base-editing efficiency for R83C correction and limited bystander editing (FIG. 9B).

### Mouse In Vivo Disease Model and Demonstration of In Vivo Correction of the R83C Single Nucleotide Mutation

### In Vivo Correction of R83C Base Editing

To validate base-editing efficiency for R83C correction in vivo, a novel GSD1a mouse that expresses the human G6PC-R83C transgene in place of mouse G6pc was generated. It was confirmed that mice homozygous for huR83C exhibited postnatal lethality and rarely survived to weaning (21 days). On glucose supplementation therapy, the animals survived to at least 3 weeks of age and revealed characteristic pathological signatures of GSD1a, such as reduced body weight, enlarged livers, significant G6Pase inhibition, and abnormal serum metabolites compared to littermate controls (FIG. 7). This phenotype is consistent with published and clinical reports in humans.

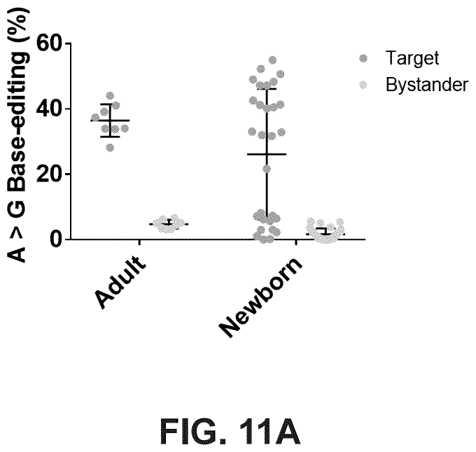

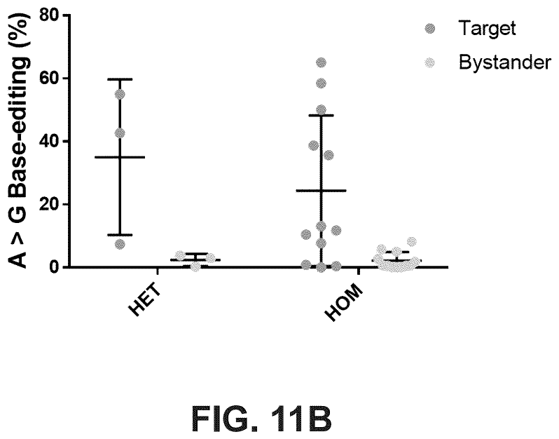

For the in vivo experiments, LNP-mediated delivery was tested in transgenic mice that were heterozygous for huR83C due to neonatal lethality of homozygous mice. The schematic in FIG. 6A depicts in vivo workflow, with lipid nanoparticle, or LNP, co-formulations of base-editor mRNA and gRNA dosed via IV injection. Given neonatal lethality of the homozygous mice, LNP-dosing was administered via the temporal vein shortly post birth, and activity was compared with that in adult mice. Next Generation Sequencing (NGS) analysis of whole liver extracts revealed approximately 40% base-editing efficiency in adults and up to ˜60% efficiency in newborns, with a broader range in efficiencies (FIG. 11A). Bystander editing remained low in adults and newborns. (FIG. 11A).

Newborn mice homozygous for huR83C were treated with lipid nanoparticles (LNP) containing guide RNA and mRNA encoding ABE. It was found that the treated mice survived and grew normally to 3 weeks of age, without hypoglycemia-induced seizures, in the absence of glucose therapy. The treated homozygous huR83C mice displayed editing efficiencies up to ˜60% in total liver extracts, consistent with littermate controls that were heterozygous for huR83C (FIG. 11B). It was thus demonstrated that LNP-mediated R83C correction was associated with the survival of the homozygous huR83C mice.

### Reversal of GSD-1a Pathology Via Base-Editing for Correction of R83C In Vivo

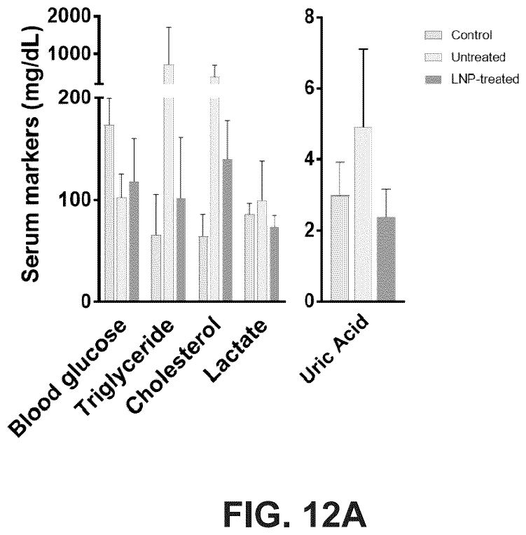

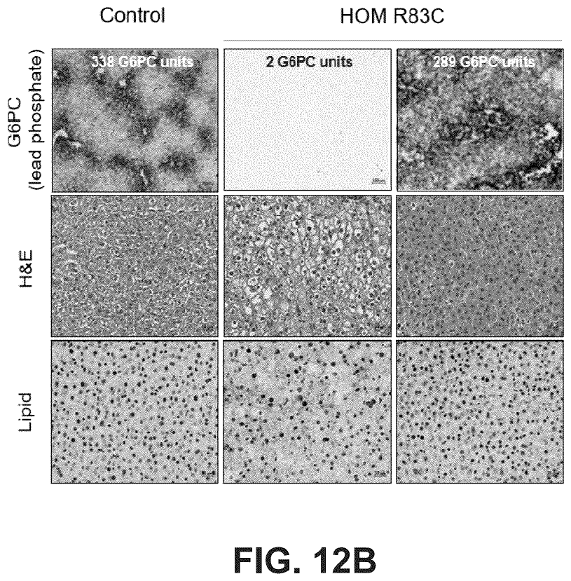

At 3 weeks, it was validated and confirmed that the treated homozygous huR83C mice displayed proper metabolic function, with restoration of near-normal serum metabolites, including glucose, triglycerides, cholesterol, lactate, and uric acid, as demonstrated by the darker-color bars in FIG. 12A, compared to controls. Moreover, the results of biochemical assays of G6PC activity (as assessed biochemically and via lead-phosphate staining) in LNP-treated homozygous huR83C mice were consistent with those of litter-mate controls. (FIG. 12A).

Hepatomegaly is another clinical presentation of GSD1a and is primarily caused by excess glycogen and lipid deposition in the liver. To evaluate the extent of hepatomegaly in homozygous huG6PC-R83C mice post base-editing, liver sections were collected from 3 wk old newborn mice and immune-histochemical analysis were conducted via hematoxylin and eosin (H&E) and Oil red O staining (FIG. 12B). Significant lipid deposition (heavy H&E staining) and enlarged hepatocytes was visualized in liver sections from homozygous mice exhibiting negligible G6Pase activity (FIG. 12B, center panels, H&E), consistent with GSD-la. In the case of base-edited homozygous huG6PC-R83C mice showing restored G6PC activity (“HOM huR83C”, right panels, FIG. 12B), lipid deposition was significantly reduced and consistent with controls (left panel), (FIG. 12B, Lipid), and restoration of hepatocyte size was apparent. Accordingly, the immuno-histochemical analyses revealed normal hepatocyte size and lipid deposition in LNP-treated mice. (FIG. 12B). Taken together, the data demonstrate the ability of base-editing to correct the R83C mutation and to reverse the metabolic defects and pathology associated with GSD1a. In addition, these data lend further support of the functional restoration and positive clinical outcomes via base-editing for GsD-1a.

As described in this Example, novel adenine base editors and guide RNA that achieved precise correction of R83C in vitro and in vivo were generated and validated. LNP-mediated delivery of ABE and gRNA yielded significant base-editing efficiency, namely, up to ˜60% base editing efficiency, with restoration of hepatic G6Pase activity and metabolic function consistent with controls.

### Single LNP Dose Administration Maintains Euglycemia During a 24 Hour Fasting Challenge Via Base Editing

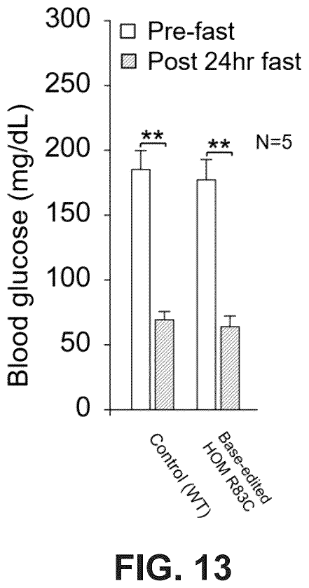

A hallmark symptom of GSD-1a pathology is fasting hypoglycemia, with a precipitous decline in blood glucose levels within minutes. A full proof-of-concept study was conducted in GSD-la transgenic mice, homozygous for huG6PC-R83C, to test whether the animals could sustain a 24 hour (hr) fast after base-editing treatment as described herein. In this study, 100% animal survival was achieved post-24 hr fasting period in LNP-treated (1.5 mpk) GSD-la animals and in healthy controls. In addition, normal fasting glucose levels were measured in control mice and in treated mice pre- and post-24 hr fasting, which maintained levels above hypoglycemic therapeutic threshold (>60 mg/dL), (FIG. 13).

G6PC Target Sequences for Use with Base Editors to Correct the R83C Mutation

In addition to the G6PC target sequence and guide RNA described in Example 1, alternative G6PC target sequences that can be used in conjunction with the base editors to effect base editing to correct the R83C mutation as described herein include those shown in Table 7. As shown, the target sequences include the types of PAMs and base editors, such as IBEs as described herein, suitable for use. In the protospacer sequences in Table 7, the position of the targeted “A” nucleotide (i.e., A8-A15) is shown in bold/underline. G6PC gRNA sequences hybridize to the complement of the G6PC target sequence shown in Table 7. The PAM sequences (e.g., SpCas9) are underlined in Table 7.

Inlaid base editors (IBEs) noted in Table 7 refer to structures of Cas9 and TadA having an architecture in which the deaminase domains are internal to (embedded inside) a CRISPR-Cas protein, e.g., Cas9. The IBE architecture allows for a greater breadth of potential base editing targets compared with other base editors and is not limited by the requirement of a suitably positioned Cas9 protospacer adjacent motif sequence. Such IBEs exhibited shifted editing windows and exhibited greater editing efficiency, thus allowing for the editing of targets outside the canonical editing window with reduced DNA and RNA off-target editing frequency. Accordingly, IBEs expand the breadth of potential base editing targets by extending the range of editing windows that can be created for any given CRISPR-Cas protein used to target the DNA. Through the insertion of the deaminase into a CRISPR protein at different strategic positions, the active site of the deaminase can be repositioned, making IBEs capable of editing outside the traditional editing window. IBE architectures are described hereinabove and in S. Haihua Chu et al., The CRISPR Journal, Vol. 4, No. 2; published online 20 Apr. 2021 (DOI: 10.1089/crispr.2020.0144).

TABLE 7
Protospacer + PAM sequences (5′ to 3′) for
correcting the R83C mutation,
where the PAM sequence is underlined

CCACCAGTATGGACACTGTC CAAA
(SEQ ID NO: 33) with spCas9-NRRH
A15 can use IBE architecture
CACCAGTATGGACACTGTCC AAAG
(SEQ ID NO: 34) with spCas9-NRRH
A14 can use IBE architecture
ACCAGTATGGACACTGTCCA AAGA
(SEQ ID NO: 35) with spCas9-NRRH
A13 can use IBE architecture
CCAGTATGGACACTGTCCAA AGAG
(SEQ ID NO: 36) with spCas9-NRRH
A12 can use IBE architecture
CAGTATGGACACTGTCCAAA GAGA
(SEQ ID NO: 37) with spCas9-NRRH
A11 can use IBE architecture
AGTATGGACACTGTCCAAAG AGA
(SEQ ID NO: 38) with spCas9-NGA
A10 can use IBE architecture
GTATGGACACTGTCCAAAGA GAAT
(SEQ ID NO: 39) with spCas9-NRRH
A9 can use IBE architecture
TATGGACACTGTCCAAAGAG AATC
(SEQ ID NO: 40) with spCas9-NRTH
A8 can use IBE architecture

The gRNA sequences which hybridize to the complement of the G6PC target sequence in Table 7 are as follows (5′ to 3′): CCACCAGUAUGGACACUGUC (SEQ ID NO: 19); CACCAGUAUGGACACUGUCC (SEQ ID NO: 20); ACCAGUAUGGACACUGUCCA (SEQ ID NO: 21); CCAGUAUGGACACUGUCCAA (SEQ ID NO: 22); CAGUAUGGACACUGUCCAAA (SEQ ID NO: 23); AGUAUGGACACUGUCCAAAG (SEQ ID NO: 24); GUAUGGACACUGUCCAAAGA (SEQ ID NO: 25); and UAUGGACACUGUCCAAAGAG (SEQ ID NO: 26).

A protospacer and PAM sequence for use in the products, compositions and methods described herein is, (5′ to 3′), CAGTATGGACACTGTCCAAAGAGAAT (SEQ ID NO: 17), in which the PAM sequence, GAGAAT, is underlined. The gRNA sequence, as presented supra, which hybridizes to the complement of the target sequence is CAGUAUGGACACUGUCCAAA (3′PAM sequence GAGAAT as shown in the sequence above) (SEQ ID NO: 2).

The gRNA sequence used in the methods described herein comprises or consists of:

(SEQ ID NO: 27)
CACCAGUAUGGACACUGUCCAAAGUUUUAGUACUCUGUAAUGAA
AAUUACAGAAUCUACUAAAACAAGGCAAAAUGCCGUGUUUAUCU
CGUCAACUUGUUGGCGAGAUUUU
or
(SEQ ID NO: 28)
CCACCAGUAUGGACACUGUCCAAAGUUUUAGUACUCUGUAAUGA
AAAUUACAGAAUCUACUAAAACAAGGCAAAAUGCCGUGUUUAUC
UCGUCAACUUGUUGGCGAGAUUUU

In some embodiments, the gRNA sequence used in the methods described herein comprises one or more modified nucleosides. Two exemplary sequences are provided below:

sgRNA_096: 23 nt protospacer
(SEQ ID NO: 29)
mCsmAsmCsCAGUAUGGACACUGUCCAAAGUUUUAGUACUCUGUA
AUGAAAAUUACAGAAUCUACUAAAACAAGGCAAAAUGCCGUGUUU
AUCUCGUCAACUUGUUGGCGAGAmUsmUsmUsU
sgRNA_097: 34 nt protospacer
(SEQ ID NO: 30)
mCsmCsmAsCCAGUAUGGACACUGUCCAAAGUUUUAGUACUCUGU
AAUGAAAAUUACAGAAUCUACUAAAACAAGGCAAAAUGCCGUGUU
UAUCUCGUCAACUUGUUGGCGAGAmUsmUsmUsU.

In context of RNA modification, “s” indicates that the preceding nucleotide possesses a 3′ phosphothioate, and “m” indicates that the following nucleotide is a 2′ OMe. For example, a nucleotide with a phosphothioate and 2′OMe has the form “mNs.” When there are two consecutive nucleotides with both a phosphothioate and 2′OMe, it is notated as “mNsmNs.”

### Example 7: Materials and Methods

Materials and methods utilized in the examples and experiments therein as described supra are set forth below.

### Animal Care

All animal studies were conducted under Taconic's Excluded Flora health standard. To sustain survival of huG6PC-R83C mice, a glucose therapy consisting of daily administered subcutaneous injections of 100-150 ul of 15% glucose per mouse. Glucose injections were not administered to mice post LNP treatment with base-editor mRNA and gRNA.

### In Vivo LNP-Dosing Work-Flow

To correct the p.R83C mutation in the huG6PC-R83C homozygous mice, LNP co-formulations of base-editor mRNA and gRNA were administered at a 1.5 mpk (milligram per kilogram) dose via the temporal vein of mice at age P1, shortly post birth. Glucose therapy was not administered to LNP-treated mice. LNP-treated mice continued to be cared for alongside littermate controls by the respective birth mother until weaning (21 days), at which point they were phenotyped. For all studies, age matched wild-type and heterozygous huG6PC-R83C littermates were used as controls. At day 21, genomic DNA harvested from livers, growth characteristics, and serum and liver markers were analyzed.

### Lipid Nanoparticle (LNP) Formulations

The base editor (mRNA encoding the base editor) and guide RNA were co-encapsulated at a 1:1 weight ratio in a lipid nanoparticle. The LNPs were generated by rapidly mixing an aqueous solution of the RNA at a pH of 3.0 with an ethanol solution containing four lipid components: an ionizable lipid, DSPC, cholesterol, and a lipid-anchored PEG. The two solutions were mixed using the benchtop microfluidics device from Precision Nanosystems. Post mixing, the formulations were dialyzed overnight at 4° C. against 1x TBS (Sigma-Aldrich, catalog #94158). They were subsequently concentrated down using 100K MWCO Amicon Ultra centrifugation tubes (Millipore Sigma, catalog #UFC910096), and filtered with 0.2 micron filters (Pall corporation, Catalog #4602). Total RNA concentration of the was determined using Quant-iT Ribogreen (ThermoFisher Scientific, catalog #R11491); particle size was determined by using the Malvern Panalytical Zetasizer.

### Next Generation Sequencing (NGS)

Next generation sequencing (NGS) was used to determine the frequency of base-edited alleles in genomic DNA from whole liver extracts of LNP-treated animals. Following LNP-treatment, mice were euthanized, and the entire liver was removed and snap frozen in liquid nitrogen. Frozen mouse livers were ground to a powder form using Geno/Grinder 2010 (Ops Diagnostics, Lebanon, NJ, USA), and genomic DNA was isolated from the liver powder using Quick Extract lysis buffer according to manufacturer's specifications. Genomic DNA was directly used in subsequent PCR amplification steps to produce a ˜170-nucleotide fragment harboring huG6PC exon 2 using the primer pair: Forward primer, GGGCATTTAAACTCCTTTGGG (SEQ ID NO: 31) and reverse primer, AGTCTCACAGGTTACAGGGA (SEQ ID NO: 32). NGS adapters were added, and the resulting amplicons were sequenced using an Illumina MiSeq instrument according to the manufacturer's instructions.

### Serum Metabolites

To measure serum metabolites, blood was collected from R83C humanized transgenic mice. Serum was then separated and extracted from whole blood, which was subsequently used for metabolite assays. For a relevant and comprehensive post-study assessment, serum glucose, serum cholesterol, and serum triglycerides were all analyzed. Serum glucose and serum cholesterol were measured using ThermoFisher Scientific (Waltham, MA, USA) Infinity Glucose Liquid Stable Reagent (Cat #: TR15421) and Infinity Cholesterol Liquid Stable Reagent (Cat #: TR13421), respectively. Serum triglycerides were measured using the Serum Triglyceride Quantification Kit (Cat #: MAK266) from Sigma-Aldrich (St. Louis, MO, USA). Uric acid was measured using the Uric Acid Liquid Stable Reagent per manufacturer specifications (Thermo Fisher Scientific (Waltham, MA, USA). Serum lactate was analyzed using the EnzyFluo L-Lactate Assay Kit from BioAssay Systems (Hayward, CA, USA).

Fasting blood glucose analysis of mice involved blood sampling via the tail vein pre- and post-24 hours after food deprivation. Blood glucose levels were measured using the HemoCue Glucose 201 System (HemoCue America, CA, USA).

Kaplan-Meier Survival Estimates for Homozygous huG6PC-R83C Mice

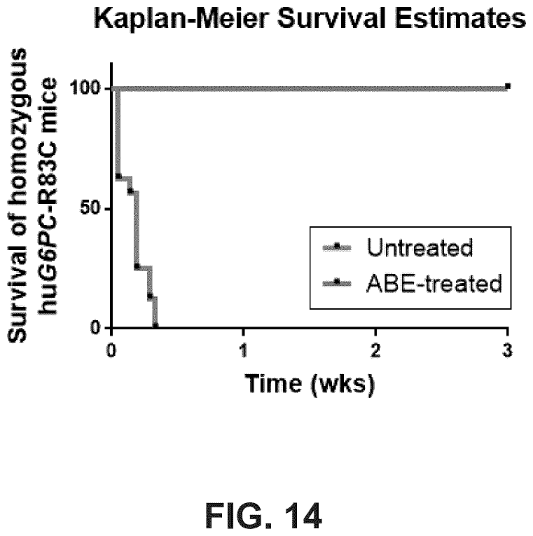

Kaplan-Meier survival curves were generated to estimate survival of newborn transgenic mice homozygous for huG6PC-R83C either post base-editing via ABE mRNA (teal) or untreated (gray, FIG. 14). Newborn mice were genotyped via PCR analysis on genomic tail DNA using the following primers, a universal forward primer (5′-ACCTACTGATGATGCACCTITGATCAATAGAT-3′(SEQ ID NO: 59)), a mouse specific reverse primer (5′-CATCACCCCTCGGGATGGTTCTT-3′(SEQ ID NO: 60)), a human specific reverse primer 1 (5′-CAGCCCAGAATCCCAACCACAAAAT-3′(SEQ ID NO: 61)), and human specific reverse primer 2 (5′-AGACCAGCTCGACTTGGGATGG-3′(SEQ ID NO: 62)). Survival was noted for transgenic mice homozygous for huG6PC-R83C. Untreated mice were either still-born (n=6) or died at 8 hrs (n=6) and 24 hrs (n=1). Administration of 15% glucose injections extended survival to 32 hrs (n=5), 48 hrs (n=2), and 56 hrs (n=2). All ABE-treated mice homozygous for huG6PC-R83C survived to termination of study at 3 wks.

### Glucose-6-Phosphatase-Alpha Activity Assay

Liver microsome isolation and microsomal phosphohydrolase assays were performed as described by Lei, K.-J., et al., 1996, Nature Genetics, 13(2):203-9. Assay methodology in Arnaotova et al. (2021, Mol. Therapy, Vol. 29, No 4) is described as follows: “Glucose-6-phosphatase dependent substrate transport in the glycogen storage disease type-1a mouse. Nat. Genet. 13, 203-209). In phosphohydrolase assays, reaction mixtures (50 uL) containing 50 mM sodium cacodylate buffer (pH 6.5), 2 mM EDTA, 10 mM Glucose-6-phosphate (G6P), and appropriate amounts of microsomal preparations were incubated at 30° C. for 10 minutes. Disrupted microsomal membranes were prepared by incubating intact membranes in 0.2% deoxycholate for 20 minutes at 4° C. Non-specific phosphatase activity was estimated by pre-incubating disrupted microsomal preparations at pH 5 for 10 minutes at 37° C. to inactivate the acid-labile G6Pase-alpha. One unit of G6Pase-alpha activity represents one nmol G6P hydrolysis per minute per mg microsomal protein. The lower level of quantitation for the microsomal G6Pase-alpha assay is 2 units.”

Enzyme histochemical analysis of G6Pase-alpha was performed as described in Lee, Y. M., Jun, H. S. Pan, C.-J. Lin, S. R., Wilson, L. H., Mansfield, B. C., and Chou, J. Y. (2012). Prevention of hepatocellular adenoma and correction of metabolic abnormalities in murine glycogen storage disease type Ia by gene therapy. Hepatology 56, 1719-1729. As described in Arnaotova et al., (2021, Mol. Therapy, Vol. 29, No 4), 10 μm-thick liver tissue sections were incubated for 10 min at room temperature in a solution containing 40 mM Tris-maleate (pH 6.5), 10 mM G6P, 300 mM sucrose, and 3.6 mM lead nitrate. After rinsing, liver sections were incubated for 2 min at room temperature in 0.09% ammonium sulfide solution, and the trapped lead phosphate was visualized following conversion to the brown-colored lead sulfide.

### Immunohistochemistry

Immunohistochemical procedures were performed as described in Arnaotova et al., 2021, Mol. Therapy, Vol. 29, No 4. In brief, H&E staining was performed on liver sections preserved in 10% neutral buffered formalin, and Oil Red O staining was performed on cryopreserved optimal cutting temperature compound (OCT) embedded liver sections following standard procedures. The stained sections were visualized using the Imager A2m microscope with Axiocam 506 camera and Zen 2.6 software (Carl Zeiss, White Plains, NY, USA).

### Example 8: NLS Promotes Nuclear Import of Guide RNA

In this example, the effect of nuclear localization signal on nuclear import of guide RNA was evaluated.

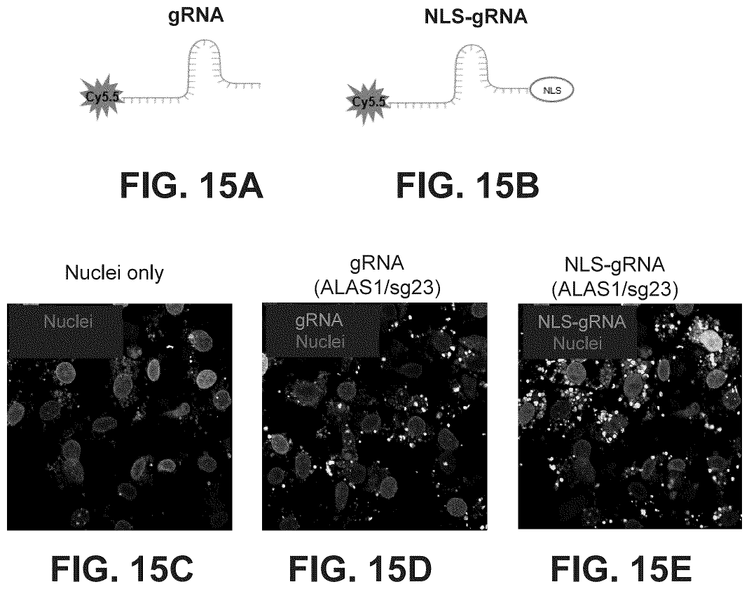

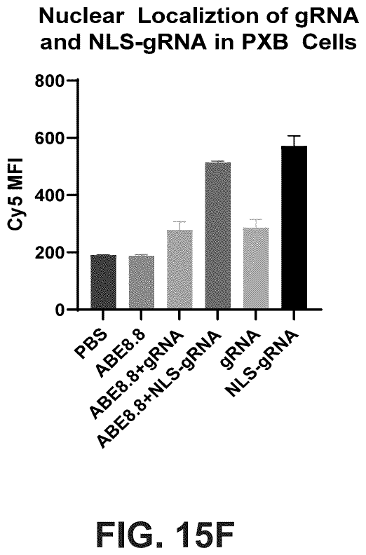

Briefly, gRNA fused to a nuclear localization signal (NLS) peptide (FIG. 15A) and a cognate control gRNA without NLS (FIG. 15B) were fluorescently labelled with a Cy5.5 dye. Human hepatocytes were lipofected with unmodified and NLS-modified gRNAs and fluorescence was measured microscopically at 24 and 48 hours post-lipofection. The nuclear envelope was counterstained blue using NucBlue stain for quantification (FIG. 15C).

The results showed that gRNA is localized to the nucleus more efficiently when conjugated to an NLS peptide (FIG. 15E) as compared to gRNA that is not conjugated to an NLS peptide (FIG. 15D).

The relative mean fluorescence intensity (MFI) was quantified as shown in FIG. 15F. The results showed that while MFI observed with ABE 8.8 alone was comparable to background fluorescence with PBS treatment, ABE 8.8 with gRNA showed an increase in fluorescence to about 300 MFI units. However, when the gRNA was conjugated to an NLS, there was an increased fluorescence of about 500 MFI units. While gRNA alone showed fluorescence of about 300 MFI units, adding NLS conjugated gRNA resulted in increased fluorescence of about 550 MFI units.

Overall, the results from this study showed that gRNA was effectively localized to the nucleus when conjugated to an NLS.

### Example 9: NLS-gRNA Shows High Potency Gene Editing in the Liver of Mice

In this example, potency of gene editing was examined in the liver of huG6PC-R83C homozygous mice administered a low dose of NLS gRNA.

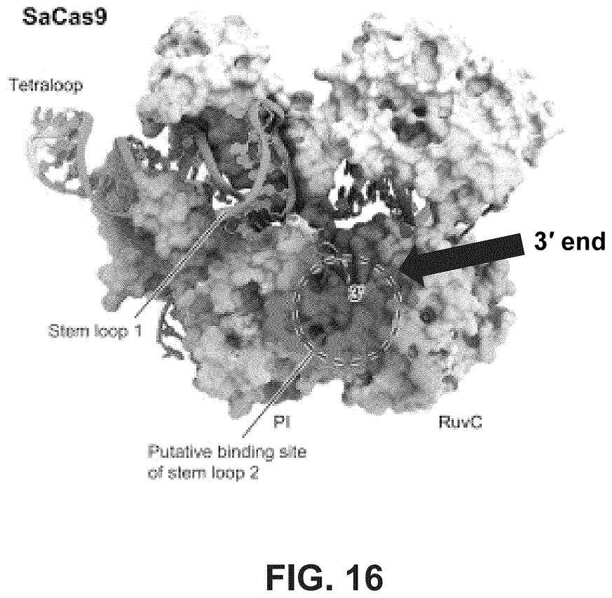

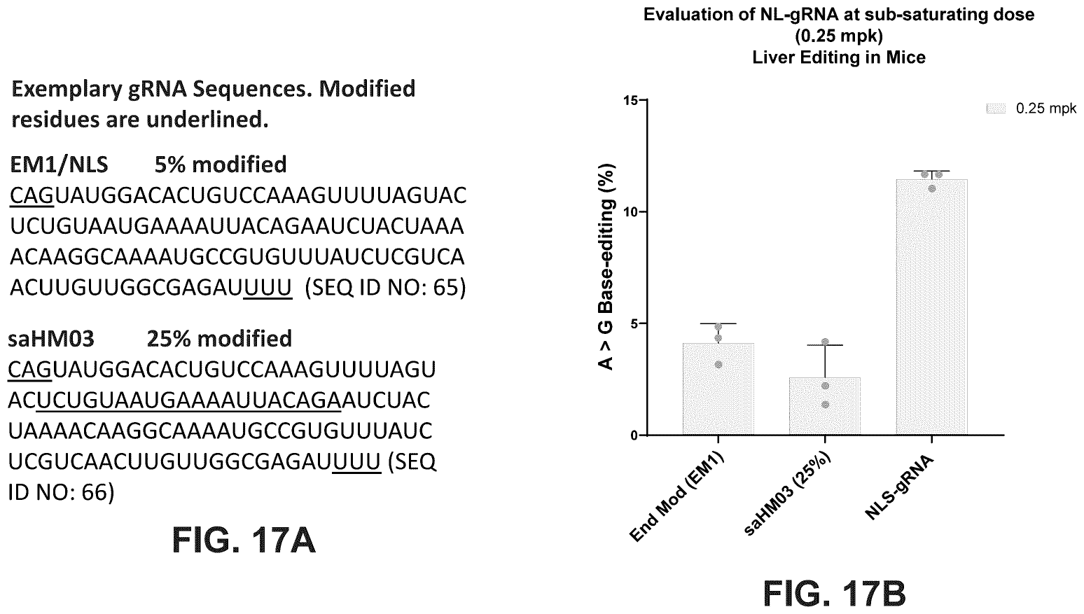

Briefly, NLS-gRNA with Type 1 end modification (EM1) was administered to correct the p.R83C mutation in the huG6PC-R83C homozygous mice, at a sub-saturating dose of 0.25 mpk (milligram per kilogram) dose via the temporal vein of mice at age P1, shortly post birth, using methods as described previously in Example 7. NLS conjugates are found to compatible with saCas9 effectors when conjugated to the 3′ terminus (FIG. 16). In this example, 5% end modified gRNA and or 25% heavy modified saHM03 gRNA were also tested in parallel. Sequences are provided below and in FIG. 17A.

TABLE 8
Exemplary end modified and heavy
modified gRNA

EM1/NLS 5% modified
CAGUAUGGACACUGUCCAAAGUUUUAGUACUCUGUAAUGAAAAUU
ACAGAAUCUACUAAAACAAGGCAAAAUGCCGUGUUUAUCUCGUCA
ACUUGUUGGCGAGAUUUU
(SEQ ID NO: 63)
saHM03 25% modified
CAGUAUGGACACUGUCCAAAGUUUUAGUACUCUGUAAUGAAAAUU
ACAGAAUCUACUAAAACAAGGCAAAAUGCCGUGUUUAUCUCGUCA
ACUUGUUGGCGAGAUUUU
(SEQ ID NO: 64)

As shown in the results in FIG. 17B, NLS-gRNA showed greater than 10% A-to-G base editing relative to less than 5% with end modified gRNA or heavy modified saHM03.

Overall, the results showed that NLS-gRNA yielded a greater than 2-fold boost in potency relative to end modified gRNA. The results demonstrated that NLS-gRNA resulted in high potency gene editing.

### Other Embodiments

From the foregoing description, it will be apparent that variations and modifications may be made to the embodiments as described herein to be adopted to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A guide RNA (gRNA) comprising a nuclear localization signal (NLS) linked to the gRNA through a linker, wherein the linker comprises a cysteine residue conjugated to the 3′ or 5′ end of the gRNA.

2. The gRNA of claim 1, wherein the gRNA comprises one or more modifications, wherein (i) one or more modifications are 2′-OMe, 2′-Fluoro, or phosphorothioate linkages; (ii) the gRNA comprises one or more modifications at the 3′ end and/or at the 5′ end; (iii) the one or more modifications occur at 1, 2, 3, 4, and/or 5 nucleotides from the 3′ end of the gRNA; (iv) the one or more modifications occur at 1, 2, 3, 4, and/or 5 nucleotides from the 5′ end of the gRNA; (v) more than 40%, 50%, 60%, 70% or 80% nucleotides of gRNA is modified; (vi) the NLS is derived from Simian Virus 40 (SV40), optionally wherein the NLS comprises an amino acid sequence of KKKRKV (SEQ ID NO: 57); (vii) the linker further comprises a peptide spacer, optionally wherein the peptide spacer comprises an amino acid sequence of KRTADGSEFESP (SEO ID NO: 58); (viii) the gRNA comprising the NLS improves base editing efficiency as compared to a gRNA without the NLS; (ix) the gRNA is a single-guide RNA (sgRNA), a tracrRNA, or a crRNA; (x) the gRNA comprises an SaCas9 backbone sequence, optionally wherein the gRNA has protospacer-adjacent motif (PAM) specificity for the nucleic acid sequence 5-NNGRRT-3′, or 5′-GAGAAT-3′, when bound to an SaCas9 or variant thereof; (xi) the gRNA comprises a nucleic acid sequence: 5′-CAGUAUGGACACUGUCCAAA-3′ (SEO ID NO: 2); and/or (xii) the gRNA comprises or consists of one of the following nucleic acid sequences:
(SEQ ID NO: 27)
CACCAGUAUGGACACUGUCCAAAGUUUUAGUACUCUGUAAUGAAA
AUUACAGAAUCUACUAAAACAAGGCAAAAUGCCGUGUUUAUCUCG
UCAACUUGUUGGCGAGAUUUU,
and
(SEQ ID NO: 28)
CCACCAGUAUGGACACUGUCCAAAGUUUUAGUACUCUGUAAUGAA
AAUUACAGAAUCUACUAAAACAAGGCAAAAUGCCGUGUUUAUCUC
GUCAACUUGUUGGCGAGAUUUU.

3.-11. (canceled)

12. The gRNA of claim 1, wherein the linker further comprises a chemical moiety that conjugates the gRNA to the peptide spacer or to the NLS, and wherein (i) the chemical moiety is covalently attached to the N-terminus of the peptide spacer or the NLS amino acid sequence, and/or the 3′ end of the gRNA; (ii) the chemical moiety is covalently attached to a cysteine residue of the peptide spacer or the NLS; and/or (iii) the chemical moiety comprises a maleimide-thiol adduct.

13.-21. (canceled)

22. A composition comprising the gRNA of claim 1 associated with or encapsulated in a lipid nanoparticle (LNP), wherein
(i) wherein the LNP further comprises an mRNA encoding a base editor; or
(ii) wherein the base editor comprises a Cas9 domain and at least one adenosine deaminase variant domain, wherein the adenosine deaminase variant domain comprises a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence, wherein the adenosine deaminase has at least about 85%, 90%, 95%, or 98% identity to said amino acid sequence
MSEVEFSHEYWMRHALTLAKRARDEREVPVGAVLVLNNRVIGEGWNRAIGLHD PTAHAEIMALROGGLVMONYRLIDATLYVTFEPCVMCAGAMIHSRIGRVVFGVRNAKT GAAGSLMDVLHYPGMNHRVEITEGILADECAALLCYFFRMPROVFNAOKKAOSSTD (SEQ ID NO: 3), or corresponding alterations in another adenosine deaminase; or
(iii) wherein the adenosine deaminase variant domain comprises any of the following combinations of alterations
a) I76Y+V82G+Y147T+Q154S;
b) L36H+V82G+Y147T+Q154S+N157K;
c) V82G+Y147D+F149Y+Q154S+D167N;
d) L36H+V82G+Y147D+F149Y+Q154S+N157K+D167N;
e) L36H+I76Y+V82G+Y147T+Q154S+N157K;
f) I76Y+V82G+Y147D+F149Y+Q154S+D167N;
g) Y147D+F149Y+D167N;
h) L36H; I76Y; V82G; Q154S; and N157K;
i) I76Y; V82G; Q154S; or
j) L36H+I76Y+V82G+Y147D+F149Y+Q154S+N157K+D167N with reference to SEQ ID NO: 3;
MSEVEFSHEYWMRHALTLAKRARDEREVPVGAVLVLNNRVIGEGWNRAIGLHD PTAHAEIMALROGGLVMONYRLIDATLYVTFEPCVMCAGAMIHSRIGRVVFGVRNAKT GAAGSLMDVLHYPGMNHRVEITEGILADECAALLCYFFRMPROVFNAOKKAOSSTD (SEQ ID NO: 3), or corresponding combinations of alterations in another adenosine deaminase; or
(iv) wherein the adenosine deaminase variant comprises the following combination of alterations I76Y+V82G+Y147D+F149Y+Q154S+D167N of SEQ ID NO: 3, or corresponding alterations in another adenosine deaminase; or
(v) wherein the Cas9 domain is a Staphylococcus aureus Cas9 (SaCas9); or
(vi) wherein the mRNA encodes a base editor comprising, consisting of, or consisting essentially of the amino acid sequence;
MSEVEFSHEYWMRHALTLAKRARDEREVPVGAVLVLNNRVIGEGWNRAIGLHD PTAHAEIMALRQGGLVMQNYRLYDATLYGTFEPCVMCAGAMIHSRIGRVVFGVRNAK TGAAGSLMDVLHYPGMNHRVEITEGILADECAALLCDFYRMPRSVFNAQKKAQSSTNS GGSSGGSSGSETPGTSESATPESSGGSSGGSKRNYILGLAIGITSVGYGIIDYETRDVIDAG VRLFKEANVENNEGRRSKRGARRLKRRRRHRIQRVKKLLFDYNLLTDHSELSGINPYEA RVKGLSQKLSEEEFSAALLHLAKRRGVHNVNEVEEDTGNELSTKEQISRNSKALEEKYV AELQLERLKKDGEVRGSINRFKTSDYVKEAKQLLKVQKAYHQLDQSFIDTYIDLLETRR TYYEGPGEGSPFGWKDIKEWYEMLMGHCTYFPEELRSVKYAYNADLYNALNDLNNLV ITRDENEKLEYYEKFQIIENVFKQKKKPTLKQIAKEILVNEEDIKGYRVTSTGKPEFTNLK VYHDIKDITARKEIIENAELLDQIAKILTIYQSSEDIQEELTNLNSELTQEEIEQISNLKGYT GTHNLSLKAINLILDELWHTNDNQIAIFNRLKLVPKKVDLSQQKEIPTTLVDDFILSPVVK RSFIQSIKVINAIIKKYGLPNDIIIELAREKNSKDAQKMINEMQKRNRQTNERIEEIIRTTGK ENAKYLIEKIKLHDMQEGKCLYSLEAIPLEDLLNNPFNYEVDHIIPRSVSFDNSFNNKVLV KQEENSKKGNRTPFQYLSSSDSKISYETFKKHILNLAKGKGRISKTKKEYLLEERDINRFS VQKDFINRNLVDTRYATRGLMNLLRSYFRVNNLDVKVKSINGGFTSFLRRKWKFKKER NKGYKHHAEDALIIANADFIFKEWKKLDKAKKVMENQMFEEKQAESMPEIETEQEYKEI FITPHQIKHIKDFKDYKYSHRVDKKPNRELINDTLYSTRKDDKGNTLIVNNLNGLYDKD NDKLKKLINKSPEKLLMYHHDPQTYQKLKLIMEQYGDEKNPLYKYYEETGNYLTKYSK KDNGPVIKKIKYYGNKLNAHLDITDDYPNSRNKVVKLSLKPYRFDVYLDNGVYKFVTV KNLDVIKKENYYEVNSKCYEEAKKLKKISNQAEFIASFYNNDLIKINGELYRVIGVNNDL LNRIEVNMIDITYREYLENMNDKRPPRIIKTIASKTQSIKKYSTDILGNLYEVKSKKHPQII KKGEGADKRTADGSEFESPKKKRKV (SEQ ID NO: 65), or an amino acid sequence at least 85% identical thereto.

23. (canceled)

24. A composition comprising the gRNA of claim 1, (i) further comprising a nuclease or an mRNA which encodes the nuclease; or
ii) further comprising a polynucleotide programmable DNA binding domain or an mRNA which encodes the polynucleotide programmable DNA binding domain; or
iii) wherein the nuclear delivery of the composition is increased by about 2 to 5 fold relative to a composition comprising gRNA without NLS, optionally wherein the gRNA comprises a sequence with at least 70% identity to any one of sequences in Table 8.

25. (canceled)

26. The composition of claim 5, wherein the composition comprises gRNA and mRNA encoding the nuclease between 1:1 and 10:1 ratio;
(ii) the composition comprises gRNA and mRNA encoding the nuclease at 1:1 ratio;
(iii) the composition comprises gRNA and mRNA encoding the nuclease at 3:1 ratio;
(iv) the composition comprises gRNA and mRNA encoding the nuclease at 9:1 ratio;
(v) the nuclease or the polynucleotide programmable DNA binding domain is a Cas protein, or further wherein the Cas protein is a Cas9 or a Cpf1, Cas12a, Cas12b, Cas12c, Cas12d, Cas12e, Cas12f, Cas12g, Cas12h, Cas12i, Cas12j, Cas12k or Cas13;
(vi) the nuclease or the polynucleotide programmable DNA binding domain is a nickase;
(vii) the nuclease or the polynucleotide programmable DNA binding domain is modified; and/or
(viii) the nuclease or the polynucleotide programmable DNA binding domain is fused to a heterologous polypeptide, or further wherein the heterologous polypeptide is a deaminase domain.

27.-36. (canceled)

37. A complex comprising
(i) a polynucleotide programmable DNA binding domain and at least one adenosine deaminase variant domain, wherein the adenosine deaminase variant domain comprises a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence, wherein the adenosine deaminase has at least about 85% identity to said amino acid sequence MSEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNAKTGAAG SLMDVLHYPGMNHRVEITEGILADECAALLCYFFRMPRQVFNAQKKAQSSTD (SEQ ID NO: 3), or corresponding alterations in another adenosine deaminase, and
(ii) the gRNA of claim 1.

38. A complex comprising
(i) a polynucleotide programmable DNA binding domain and at least one adenosine deaminase variant domain wherein the adenosine deaminase variant domain comprises any of the following combinations of alterations
a) I76Y+V82G+Y147T+Q154S;
b) L36H+V82G+Y147T+Q154S+N157K;
c) V82G+Y147D+F149Y+Q154S+D167N;
d) L36H+V82G+Y147D+F149Y+Q154S+N157K+D167N;
e) L36H+I76Y+V82G+Y147T+Q154S+N157K;
f) I76Y+V82G+Y147D+F149Y+Q154S+D167N;
g) Y147D+F149Y+D167N;
h) L36H; I76Y; V82G; Q154S; and N157K;
i) I76Y; V82G; Q154S; or
j) L36H+I76Y+V82G+Y147D+F149Y+Q154S+N157K+D167N with reference to SEQ ID NO: 3: MSEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNAKTGAAG SLMDVLHYPGMNHRVEITEGILADECAALLCYFFRMPRQVFNAQKKAQSSTD (SEQ ID NO: 3), or corresponding combinations of alterations in another adenosine deaminase; and
(ii) the gRNA of claim 1.

39. The complex of claim 37, wherein (i) the adenosine deaminase has at least about 90% or about 95% identity to SEQ ID NO: 3; (ii) the adenosine deaminase comprises or consists essentially of SEQ ID NO: 3; (iii) the adenosine deaminase variant comprises the following combination of alterations I76Y+V82G+Y147D+F149Y+Q154S+D167N of SEQ ID NO: 3, or corresponding alterations in another adenosine deaminase; (iv) the polynucleotide programmable DNA binding domain is a Cas9, or further wherein the Cas9 comprises a nuclease dead Cas9 (dCas9), a Cas9 nickase (nCas9), or a nuclease active Cas9 and/or wherein the Cas9 is a Staphylococcus aureus Cas9 (SaCas9), Streptococcus thermophilus 1 Cas9 (St1Cas9), a Streptococcus pyogenes Cas9 (SpCas9), or variants thereof; and/or
(v) the adenosine deaminase variant domain is internal to the polynucleotide programmable DNA binding domain.

40.-45. (canceled)

46. A pharmaceutical composition comprising the gRNA of claim 1 and a pharmaceutically acceptable carrier.

47. (canceled)

48. A composition comprising an engineered or non-naturally occurring CRISPR associated Cas (CRISPR-Cas) system comprising:
(a) a Cas protein;
(b) a gRNA comprising a nuclear localization signal (NLS) linked to the gRNA through a linker;
wherein the linker comprises a cysteine residue conjugated to the 3′ end of the gRNA; and
wherein the gRNA is capable of forming a complex with a Cas protein and targeting the Cas protein to a target DNA.

49. The composition of claim 48, wherein the Cas protein is fused to a heterologous polypeptide, or further wherein the heterologous polypeptide is a deaminase domain.

50. (canceled)

51. The composition of claim 46, wherein the deaminase variant domain comprises a glycine (G) at amino acid position 82, a threonine (T) or an aspartic acid (D) at amino acid position 147, a serine (S) at amino acid position 154, and one or more of a histidine (H) at amino acid position 36, a tyrosine at amino acid position 76, a tyrosine at amino acid position 149, a lysine (K) at amino acid position 157, and an asparagine (N) at amino acid position 167 of the following amino acid sequence, wherein the adenosine deaminase has at least about 85% identity to said amino acid sequence MSEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNAKTGAAG SLMDVLHYPGMNHRVEITEGILADECAALLCYFFRMPRQVFNAQKKAQSSTD (SEQ ID NO: 3), or corresponding alterations in another adenosine deaminase; or further wherein the deaminase variant domain comprises any of the following combinations of alterations
a) I76Y+V82G+Y147T+Q154S;
b) L36H+V82G+Y147T+Q154S+N157K;
c) V82G+Y147D+F149Y+Q154S+D167N;
d) L36H+V82G+Y147D+F149Y+Q154S+N157K+D167N;
e) L36H+I76Y+V82G+Y147T+Q154S+N157K;
f) I76Y+V82G+Y147D+F149Y+Q154S+D167N;
g) Y147D+F149Y+D167N;
h) L36H; I76Y; V82G; Q154S; and N157K;
i) I76Y; V82G; Q154S; or
j) L36H+I76Y+V82G+Y147D+F149Y+Q154S+N157K+D167N with reference to SEQ ID NO: 3: MSEVEFSHEYWMRHALTLAKRARDEREVPVGAVLVLNNRVIGEGWNRAIGLHDPTAH AEIMALRQGGLVMQNYRLIDATLYVTFEPCVMCAGAMIHSRIGRVVFGVRNAKTGAAG SLMDVLHYPGMNHRVEITEGILADECAALLCYFFRMPRQVFNAQKKAQSSTD (SEQ ID NO: 3), or corresponding combinations of alterations in another adenosine deaminase; or further wherein:
i) the adenosine deaminase has at least about 90% identity to SEQ ID NO: 3;
ii) the adenosine deaminase variant comprises the following combination of alterations I76Y+V82G+Y147D+F149Y+Q154S+D167N of SEQ ID NO: 3, or corresponding alterations in another adenosine deaminase;
iii) the gRNA comprises a nucleic acid sequence: 5′-CAGUAUGGACACUGUCCAAA-3′ (SEQ ID NO: 2);
iv) the adenosine deaminase variant domain is internal to the Cas protein; and/or
v) the gRNA comprises one or more modifications, or further wherein one or more modifications are 2′-OMe, 2′-Fluoro, or phosphorothioate linkages.

52.-66. (canceled)

67. The composition of claim 51, wherein (i) more than 80% of nucleotides of gRNA is modified; (ii) the NLS is derived from Simian Virus 40 (SV40), (iii) the NLS comprises an amino acid sequence of KKKRKV (SEQ ID NO: 57); (iv) the linker further comprises a peptide spacer, (v) the peptide spacer further comprises an amino acid sequence of KRTADGSEFESP (SEQ ID NO: 58); (vi) the linker further comprises a chemical moiety that conjugates the gRNA to the peptide spacer or to the NLS; and/or (vii) the gRNA comprising the NLS improves base editing efficiency as compared to a gRNA without the NLS.

68.-74. (canceled)

75. A method of treating a genetic disease in a subject in need thereof, the method comprising administering to the subject the gRNA of claim 1.

76. A method of treating Glycogen Storage Disease Type 1a (GSD1a), the method comprising administering to the subject the gRNA of claim 1 or that hybridize to the complement of a G6PC target sequence in Table 7, wherein the gRNA targets one or more of organs selected from liver, kidney, brain and heart.

77. The composition of claim 48, wherein the Cas9 protein is saCas9, wherein an adenosine deaminase variant is fused to Cas9 protein, and
wherein the adenosine deaminase variant comprises V82G, Y147T/D, Q154S, and one or more of L36H, I76Y, F149Y, N157K, and D167N with reference to SEQ ID NO: 3; and
wherein the gRNA comprises SEQ ID NO: 2.

78. A method of modifying a target nucleic acid in a cell, or altering expression of a target nucleic acid in a eukaryotic cell, comprising:
contacting the cell with a nuclease, and a gRNA of claim 1, wherein the gRNA comprises a direct repeat sequence and a spacer sequence capable of hybridizing to the target nucleic acid, and
wherein the Cas9 protein is capable of binding to the gRNA and of causing a modification in the target nucleic acid sequence complementary to the gRNA, wherein the method results in base editing of a gene.

79.-80. (canceled)

81. An engineered, non-naturally occurring CRISPR-Cas system comprising the gRNA of claim 1.

82. A method of making a guide RNA comprising a nuclear localization signal (NLS) comprising:
contacting the gRNA comprising an amine group at a 3′ end with a peptide comprising the NLS sequence and a cysteine residue at the N-terminus such that gRNA is conjugated to the NLS.

83.-88. (canceled)


SEQUENCE LISTING (65 sequences)

SEQ ID NO: 1 - DNA, 26 bp, Homo sapiens
  source 1..26: mol_type = genomic DNA
cagtatggacactgtccaaagagaat

SEQ ID NO: 2 - skipped

SEQ ID NO: 3 - protein, 167 aa, unidentified
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 4 - protein, 17 aa, synthetic construct
MAPKKKRKVGIHGVPAA

SEQ ID NO: 5 - DNA, 51 bp, synthetic construct
  source 1..51: mol_type = other DNA
atggccccaaagaagaagcggaaggtcggtatccacggagtcccagcagcc

SEQ ID NO: 6 - protein, 167 aa, unidentified
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 7 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 8 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 9 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 10 - protein, 183 aa, unidentified
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 11 - protein, 164 aa, unidentified
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 12 - protein, 171 aa, Haemophilus influenzae
MDAAKVRSEFDEKMMRYALELADKAEALGEIPVGAVLVDDARNIIGEGWNLSIVQSDPTAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 13 - protein, 161 aa, unidentified
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 14 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 15 - protein, 167 aa, unidentified
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCTFFRMPRQVFNAQKKAQSSTD

SEQ ID NO: 16 - protein, 167 aa, unidentified; identical to SEQ ID NO: 15

SEQ ID NO: 17 - DNA, 30 bp, synthetic construct
  source 1..30: mol_type = other DNA
ccaccagtatggacactgtccaaagagaat

SEQ ID NO: 18 - protein, 10 aa, synthetic construct
WWYPCQGFLI

SEQ ID NO: 19 - DNA, 20 bp, synthetic construct
  source 1..20: mol_type = other DNA
ccaccagtatggacactgtc

SEQ ID NO: 20 - RNA, 20 nt, synthetic construct
  source 1..20: mol_type = other RNA
caccagtatggacactgtcc

SEQ ID NO: 21 - RNA, 20 nt, synthetic construct
  source 1..20: mol_type = other RNA
accagtatggacactgtcca

SEQ ID NO: 22 - RNA, 20 nt, synthetic construct
  source 1..20: mol_type = other RNA
ccagtatggacactgtccaa

SEQ ID NO: 23 - RNA, 20 nt, synthetic construct
  source 1..20: mol_type = other RNA
cagtatggacactgtccaaa

SEQ ID NO: 24 - RNA, 20 nt, synthetic construct
  source 1..20: mol_type = other RNA
agtatggacactgtccaaag

SEQ ID NO: 25 - RNA, 20 nt, synthetic construct
  source 1..20: mol_type = other RNA
gtatggacactgtccaaaga

SEQ ID NO: 26 - RNA, 20 nt, synthetic construct
  source 1..20: mol_type = other RNA
tatggacactgtccaaagag

SEQ ID NO: 27 - RNA, 111 nt, synthetic construct
  source 1..111: mol_type = other RNA
caccagtatggacactgtccaaagttttagtactctgtaatgaaaattacagaatctactaaaacaaggcaaaatgccgtgtttatctcgtcaacttgttggcgagatttt

SEQ ID NO: 28 - RNA, 112 nt, synthetic construct
  source 1..112: mol_type = other RNA
ccaccagtatggacactgtccaaagttttagtactctgtaatgaaaattacagaatctactaaaacaaggcaaaatgccgtgtttatctcgtcaacttgttggcgagatttt

SEQ ID NO: 29 - RNA, 123 nt, synthetic construct
  source 1..123: mol_type = other RNA
mcsmasmcscagtatggacactgtccaaagttttagtactctgtaatgaaaattacagaatctactaaaacaaggcaaaatgccgtgtttatctcgtcaacttgttggcgagamtsmtsmtst

SEQ ID NO: 30 - RNA, 124 nt, synthetic construct
  source 1..124: mol_type = other RNA
mcsmcsmasccagtatggacactgtccaaagttttagtactctgtaatgaaaattacagaatctactaaaacaaggcaaaatgccgtgtttatctcgtcaacttgttggcgagamtsmtsmtst

SEQ ID NO: 31 - DNA, 20 bp, synthetic construct
  source 1..20: mol_type = other DNA
gggcattaaactcctttggg

SEQ ID NO: 32 - DNA, 20 bp, synthetic construct
  source 1..20: mol_type = other DNA
agtctcacaggttacaggga

SEQ ID NO: 33 - RNA, 24 nt, synthetic construct
  source 1..24: mol_type = other RNA
ccaccagtatggacactgtccaaa

SEQ ID NO: 34 - RNA, 24 nt, synthetic construct
  source 1..24: mol_type = other RNA
caccagtatggacactgtccaaag

SEQ ID NO: 35 - RNA, 24 nt, synthetic construct
  source 1..24: mol_type = other RNA
accagtatggacactgtccaaaga

SEQ ID NO: 36 - RNA, 24 nt, synthetic construct
  source 1..24: mol_type = other RNA
ccagtatggacactgtccaaagag

SEQ ID NO: 37 - RNA, 24 nt, synthetic construct
  source 1..24: mol_type = other RNA
cagtatggacactgtccaaagaga

SEQ ID NO: 38 - RNA, 23 nt, synthetic construct
  source 1..23: mol_type = other RNA
agtatggacactgtccaaagaga

SEQ ID NO: 39 - RNA, 24 nt, synthetic construct
  source 1..24: mol_type = other RNA
gtatggacactgtccaaagagaat

SEQ ID NO: 40 - RNA, 24 nt, synthetic construct
  source 1..24: mol_type = other RNA
tatggacactgtccaaagagaatc

SEQ ID NO: 41 - protein, 7 aa, synthetic construct
PKKKRKV

SEQ ID NO: 42 - protein, 16 aa, synthetic construct
KRPAATKKAGQAKKKK

SEQ ID NO: 43 - protein, 9 aa, synthetic construct
PAAKRVKLD

SEQ ID NO: 44 - protein, 11 aa, synthetic construct
RQRRNELKRSP

SEQ ID NO: 45 - protein, 38 aa, synthetic construct
NQSSNFGPMKGGNFGGRSSGPYGGGGQYFAKPRNQGGY

SEQ ID NO: 46 - protein, 42 aa, synthetic construct
RMRIZFKNKGKDTAELRRRRVEVSVELRKAKKDEQILKRRNV

SEQ ID NO: 47 - protein, 8 aa, synthetic construct
VSRKRPRP

SEQ ID NO: 48 - protein, 8 aa, synthetic construct
PPKKARED

SEQ ID NO: 49 - protein, 8 aa, synthetic construct
POPKKKPL

SEQ ID NO: 50 - protein, 12 aa, synthetic construct
SALIKKKKKMAP

SEQ ID NO: 51 - protein, 5 aa, synthetic construct
DRLRR

SEQ ID NO: 52 - protein, 7 aa, synthetic construct
PKQKKRK

SEQ ID NO: 53 - protein, 10 aa, synthetic construct
RKLKKKIKKL

SEQ ID NO: 54 - protein, 10 aa, synthetic construct
REKKKFLKRR

SEQ ID NO: 55 - protein, 20 aa, synthetic construct
KRKGDEVDGVDEVAKKKSKK

SEQ ID NO: 56 - protein, 17 aa, synthetic construct
RKCLQAGMNLEARKTKK

SEQ ID NO: 57 - protein, 6 aa, synthetic construct
KKKRKV

SEQ ID NO: 58 - protein, 12 aa, synthetic construct
KRTADGSEFESP

SEQ ID NO: 59 - DNA, 32 bp, synthetic construct
  source 1..32: mol_type = other DNA
acctactgatgatgcacctttgatcaatagat

SEQ ID NO: 60 - DNA, 23 bp, synthetic construct
  source 1..23: mol_type = other DNA
catcacccctcgggatggttctt

SEQ ID NO: 61 - DNA, 25 bp, synthetic construct
  source 1..25: mol_type = other DNA
cagcccagaatcccaaccacaaaat

SEQ ID NO: 62 - DNA, 22 bp, synthetic construct
  source 1..22: mol_type = other DNA
agaccagctcgacttgggatgg

SEQ ID NO: 63 - RNA, 108 nt, synthetic construct
  source 1..108: mol_type = other RNA
cagtatggacactgtccaaagttttagtactctgtaatgaaaattacagaatctactaaaacaaggcaaaatgccgtgtttatctcgtcaacttgttggcgagatttt

SEQ ID NO: 64 - RNA, 108 nt, synthetic construct; identical to SEQ ID NO: 63
  source 1..108: mol_type = other RNA

SEQ ID NO: 65 - protein, 1273 aa, synthetic construct
MSEVEFSHEYWMRHALTLAKRARDEREVPVGAVLVLNNRVIGEGWNRAIGLHDPTAHAEIMALRQGGLVMQNYRLYDATLYGTFEPCVMCAGAMIHSRIGRVVFGVRNAKTGAAGSLMDVLHYPGMNHRVEITEGILADECAALLCDFYRMPRSVFNAQKKAQSSTNSGGSSGGSSGSETPGTSESATPESSGGSSGGSKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGEGADKRTADGSEFESPKKKRKV